(19) United States
(12) Patent Application Publication
(10) Pub. No.: US 2006/0246422 A1
(43) Pub. Date: Nov. 2, 2006

(54) Methods and compositions for use in spliceosome mediated RNA trans-splicing

(76) Inventors: Lloyd G. Mitchell, Durham, NC (US); Mariano A. Garcia-Blanco, Durham, NC (US); Carl C. Baker, Potomac, MD (US); Madaiah Puttaraju, Durham, NC (US)

Correspondence Address: ARENT FOX PLLC, 1675 BROADWAY, NEW YORK, NY, 10019, US

(21) Appl. No.: 11/256,585

(22) Filed: Oct. 21, 2005

Related U.S. Application Data
(63) Continuation of application No. 09/941,492, filed on Aug. 29, 2001, now abandoned.
Application No. 09/941,492 is a continuation-in-part of application No. 09/838,858, filed on Apr. 20, 2001, now abandoned.
Application No. 09/838,858 is a continuation-in-part of application No. 09/756,096, filed on Jan. 8, 2001.
Application No. 09/756,096 is a continuation-in-part of application No. 09/158,863, filed on Sep. 23, 1998, now Pat. No. 6,280,978.
Application No. 09/158,863 is a continuation-in-part of application No. 09/133,717, filed on Aug. 13, 1998, now Pat. No. 6,083,702.
Application No. 09/133,717 is a continuation-in-part of application No. 09/087,233, filed on May 28, 1998, now abandoned.
Application No. 09/087,233 is a continuation-in-part of application No. 08/766,354, filed on Dec. 13, 1996, now Pat. No. 6,013,487.
(60) Provisional application No. 60/008,317, filed on Dec. 7, 1995.

(51) Int. Cl. C12Q 1/70; C12Q 1/68; C07H 21/04; A61K 48/00; C12N 15/86; C12N 15/74; C12N 1/21

(52) U.S. Cl. 435/5; 435/6; 435/456; 435/252.3; 435/472; 435/235.1; 536/23.72; 514/44

(57) ABSTRACT

The molecules and methods of the present invention provide a means for in vivo production of a trans-spliced molecule in a selected subset of cells. The pre-trans-splicing molecules of the invention are substrates for a trans-splicing reaction between the pre-trans-splicing molecules and a pre-mRNA that is uniquely expressed in the specific target cells. The in vivo trans-splicing reaction provides a novel mRNA that is functional as mRNA or encodes a protein to be expressed in the target cells. The expression product of the mRNA is a protein of therapeutic value to the cell or host organism, a toxin that kills the specific cells or a novel protein not normally present in such cells. The invention further provides PTMs that have been genetically engineered for the identification of exon/intron boundaries of pre-mRNA molecules using an exon tagging method.

### CROSS REFERENCE TO RELATED APPLICATION

The present application is a continuation of continuation-in-part pending application Ser. No. 09/941,492 filed Aug. 29, 2001 which is a continuation-in-part of pending application Ser. No. 09/838,858 filed Apr. 20, 2001 which is a continuation-in-part of pending application Ser. No. 09/756,096 filed Jan. 8, 2001 which is a continuation-in-part of pending application Ser. No. 09/158,863 filed Sep. 23, 1998 which is a continuation-in-part of Ser. No. 09/133,717 filed on Aug. 13, 1998 which is a continuation-in-part of Ser. No. 09/087,233 filed on May 28, 1998, which is a continuation-in-part of pending application Ser. No. 08/766,354 filed on Dec. 13, 1996, which claims benefit to provisional application No. 60/008,317 filed on Dec. 15, 1995.

### STATEMENT REGARDING FEDERALLY SPONSORED RESEARCH

The present invention was made with government support under Grant Nos. SBIR R43DK56526-01 and SBIR R44DK56526-02. The government has certain rights in the invention.

### INTRODUCTION

The present invention provides methods and compositions for generating novel nucleic acid molecules through targeted spliceosomal trans-splicing. The compositions of the invention include pre-trans-splicing molecules (PTMs) designed to interact with a natural target precursor messenger RNA molecule (target pre-mRNA) and mediate a trans-splicing reaction resulting in the generation of a novel chimeric RNA molecule (chimeric RNA). The PTMs of the invention are genetically engineered so as to result in the production of a novel chimeric RNA which may itself perform a function, such as inhibiting the translation of the RNA, or that encodes a protein that complements a defective or inactive protein in a cell, or encodes a toxin which kills specific cells. Generally, the target pre-mRNA is chosen as a target because it is expressed within a specific cell type thus providing a means for targeting expression of the novel chimeric RNA to a selected cell type. The invention further relates to PTMs that have been genetically engineered for the identification of exon/intron boundaries of pre-mRNA molecules using an exon tagging method. In addition, PTMs can be designed to result in the production of chimeric RNA encoding for peptide affinity purification tags which can be used to purify and identify proteins expressed in a specific cell type. The methods of the invention encompass contacting the PTMs of the invention with a target pre-mRNA under conditions in which a portion of the PTM is trans-spliced to a portion of the target pre-mRNA to form a novel chimeric RNA molecule. The methods and compositions of the invention can be used in cellular gene regulation, gene repair and suicide gene therapy for treatment of proliferative disorders such as cancer or treatment of genetic, autoimmune or infectious diseases. In addition, the methods and compositions of the invention can be used to generate novel nucleic acid molecules in plants through targeted splicesomal trans-splicing. For example, targeted trans-splicing may be used to regulate gene expression in plants for treatment of plants diseases, engineering of disease resistant plants or expression of desirable genes in plants. The methods and compositions of the invention can also be used to map intron-exon boundaries and to identify novel proteins expressed in any given cell.

### BACKGROUND OF THE INVENTION

DNA sequences in the chromosome are transcribed into pre-mRNAs which contain coding regions (exons) and generally also contain intervening non-coding regions (introns). Introns are removed from pre-mRNAs in a precise process called splicing (Chow et al., 1977, Cell 12:1-8; and Berget, S. M. et al., 1977, Proc. Natl. Acad. Sci. USA 74:3171-3175). Splicing takes place as a coordinated interaction of several small nuclear ribonucleoprotein particles (snRNP's) and many protein factors that assemble to form an enzymatic complex known as the spliceosome (Moore et al., 1993, in The RNA World, R. F. Gestland and J. F. Atkins eds. (Cold Spring Harbor Laboratory Press, Cold Spring Harbor, N.Y.); Kramer, 1996, Annu. Rev. Biochem., 65:367-404; Staley and Guthrie, 1998, Cell 92:315-326).

Pre-mRNA splicing proceeds by a two-step mechanism. In the first step, the 5′ splice site is cleaved, resulting in a “free” 5′ exon and a lariat intermediate (Moore, M. J. and P. A. Sharp, 1993, Nature 365:364-368). In the second step, the 5′ exon is ligated to the 3′ exon with release of the intron as the lariat product. These steps are catalyzed in a complex of small nuclear ribonucleoproteins and proteins called the spliceosome.

The splicing reaction sites are defined by consensus sequences around the 5′ and 3′ splice sites. The 5′ splice site consensus sequence is AG/GURAGU (where A=adenosine, U=uracil, G=guanine, C=cytosine, R=purine and /=the splice site). The 3′ splice region consists of three separate sequence elements: the branch point or branch site, a polypyrimidine tract and the 3′ splice consensus sequence (YAG). These elements loosely define a 3′ splice region, which may encompass 100 nucleotides of the intron upstream of the 3′ splice site. The branch point consensus sequence in mammals is YNYURAC (where N=any nucleotide, Y=pyrimidine). The underlined A is the site of branch formation (the BPA=branch point adenosine). The 3′ splice consensus sequence is YAG/G. Between the branch point and the splice site there is usually found a polypyrimidine tract, which is important in mammalian systems for efficient branch point utilization and 3′ splice site recognition (Roscigno, R., F. et al., 1993, J. Biol. Chem. 268:11222-11229). The first YAG trinucleotide downstream from the branch point and polypyrimidine tract is the most commonly used 3′ splice site (Smith, C. W. et al., 1989, Nature 342:243-247).

In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Nat'l. Acad. Sci. USA, 87:8879; Davis et al., 1995, J. Biol. Chem. 270:21813) and in plant mitochondria (Malek et al., 1997, Proc. Nat'l. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cis-splicing. In addition, sequences at the 3′ splice site and some of the snRNPs which catalyze the trans-splicing reaction, closely resemble their counterparts involved in cis-splicing.

Trans-splicing may also refer to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Nat'l. Acad. Sci. USA 86:8020). In addition, trans-splicing of c-myb pre-RNA has been demonstrated (Vellard, M. et al. Proc. Nat'l. Acad. Sci., 1992 89:2511-2515) and more recently, RNA transcripts from cloned SV40 trans-spliced to each other were detected in cultured cells and nuclear extracts (Eul et al., 1995, EMBO. J. 14:3226). However, naturally occurring trans-splicing of mammalian pre-mRNAs is thought to be an exceedingly rare event.

In vitro trans-splicing has been used as a model system to examine the mechanism of splicing by several groups (Konarska & Sharp, 1985, Cell 46:165-171 Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base pairing was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Nat'l. Acad. Sci. USA 92:7056-7059). These reactions occur at relatively low frequencies and require specialized elements, such as a downstream 5′ splice site or exonic splicing enhancers.

In addition to splicing mechanisms involving the binding of multiple proteins to the precursor mRNA which then act to correctly cut and join RNA, a third mechanism involves cutting and joining of the RNA by the intron itself, by what are termed catalytic RNA molecules or ribozymes. The cleavage activity of ribozymes has been targeted to specific RNAs by engineering a discrete “hybridization” region into the ribozyme. Upon hybridization to the target RNA, the catalytic region of the ribozyme cleaves the target. It has been suggested that such ribozyme activity would be useful for the inactivation or cleavage of target RNA in vivo, such as for the treatment of human diseases characterized by production of foreign of aberrant RNA. The use of antisense RNA has also been proposed as an alternative mechanism for targeting and destruction of specific RNAs. In such instances small RNA molecules are designed to hybridize to the target RNA and by binding to the target RNA prevent translation of the target RNA or cause destruction of the RNA through activation of nucleases.

Until recently, the practical application of targeted trans-splicing to modify specific target genes has been limited to group I ribozyme-based mechanisms. Using the Tetrahymena group I ribozyme, targeted trans-splicing was demonstrated in E. coli.coli (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596). While many applications of targeted RNA trans-splicing driven by modified group I ribozymes have been explored, targeted trans-splicing mediated by native mammalian splicing machinery, i.e., spliceosomes, has not been previously reported.

### SUMMARY OF THE INVENTION

The present invention relates to compositions and methods for generating novel nucleic acid molecules through spliceosome-mediated targeted trans-splicing. The compositions of the invention include pre-trans-splicing molecules (hereinafter referred to as “PTMs”) designed to interact with a natural target pre-mRNA molecule (hereinafter referred to as “pre-mRNA”) and mediate a spliceosomal trans-splicing reaction resulting in the generation of a novel chimeric RNA molecule (hereinafter referred to as “chimeric RNA”). The methods of the invention encompass contacting the PTMs of the invention with a natural target pre-mRNA under conditions in which a portion of the PTM is spliced to the natural pre-mRNA to form a novel chimeric RNA. The PTMs of the invention are genetically engineered so that the novel chimeric RNA resulting from the trans-splicing reaction may itself perform a function such as inhibiting the translation of RNA, or alternatively, the chimeric RNA may encode a protein that complements a defective or inactive protein in the cell, or encodes a toxin which kills the specific cells. Generally, the target pre-mRNA is chosen because it is expressed within a specific cell type thereby providing a means for targeting expression of the novel chimeric RNA to a selected cell type. The target cells may include, but are not limited to those infected with viral or other infectious agents, benign or malignant neoplasms, or components of the immune system which are involved in autoimmune disease or tissue rejection. The PTMs of the invention may also be used to correct genetic mutations found to be associated with genetic diseases. In particular, double-trans-splicing reactions can be used to replace internal exons. The PTMs of the invention can also be genetically engineered to tag exon sequences in a mRNA molecule as a method for identifying intron/exon boundaries in target pre-mRNA. The invention further relates to the use of PTM molecules that are genetically engineered to encode a peptide affinity purification tag for use in the purification and identification of proteins expressed in a specific cell type. The methods and compositions of the invention can be used in gene regulation, gene repair and targeted cell death. Such methods and compositions can be used for the treatment of various diseases including, but not limited to, genetic, infectious or autoimmune diseases and proliferative disorders such as cancer and to regulate gene expression in plants.

### BRIEF DESCRIPTION OF THE DRAWINGS

FIG. 1A. Model of Pre-Trans-splicing RNA.

FIG. 1B. Model PTM constructs and targeted trans-splicing strategy. Schematic representation of the first generation PTMs (PTM+Sp and PTM−Sp). BD, binding domain; NBD, non-binding domain; BP, branch point; PPT, pyrimidine tract; ss, splice site and DT-A, diphtheria toxin subunit A. Unique restriction sites within the PTMS are indicated by single letters: E; EcoRI; X, Xhol; K, Kpnl; P, Pstl; A, Accl; B, BamHI and H; HindIII.

FIG. 1C. Schematic drawing showing the binding of PTM+Sp via conventional Watson Crick base pairing to the βHCG6 target pre-mRNA and the proposed cis- and trans-splicing mechanism.

FIG. 2A. In vitro trans-splicing efficiency of various PTM constructs into βHCG6 target. A targeted binding domain and active splice sites correlate with PTM trans-splicing activity. Full length targeted (pcPTM+Sp), non-targeted (PTM−Sp) and the splice mutants [Py(−)AG(−) and BP(−)Py(−)AG(−)] PTM RNAs were added to splicing reactions containing βHCG6 target pre-mRNA. The products were RT-PCR amplified using primers βHCG-F (specific for target βHCG6 exon 1) and DT-5R (complementary to DT-A) and analyzed by electrophoresis in a 1.5% agarose gel.

FIG. 2B. In vitro trans-splicing efficiency of various PTM constructs. Full length PTM with a spacer between the binding domain and splice site (PTM+Sp), PTM without the spacer region (PTM+) and short PTMs that contain a target binding domain (short PTM+) or a non-target binding region (PTM−) were added to splicing reactions containing βHCG target pre-mRNA. The products were RT-PCR amplified using primers βHCG-F and DT-3. For reactions containing the short PTMs, the reverse PCR primer was DT-4, since the binding site for DT-3 was removed from the PTM.

FIG. 3. Nucleotide sequence demonstrating the in vitro trans-spliced product between a PTM and target pre-mRNA (SEQ ID NO: 53). The 466 bp trans-spliced RT-PCR product from FIG. 2 (lane 2) was re-amplified using a 5′ biotin labeled forward primer (βHCG-F) and a nested unlabeled reverse primer (DT-3R). Single stranded DNA was purified and sequenced directly using toxin specific DT-3R primer. The arrow indicates the splice junction between the last nucleotide of target βHCG6 exon I and the first nucleotide encoding DT-A.

FIG. 4A. Schematic diagram of the “safety” PTM and variations, demonstrating the PTM intramolecular base-paired stem, intended to mask the BP and PPT from splicing factors (SEQ ID NOS: 54, 55, 56). Underlined sequences represent the βHCG6 intron 1 complementary target-binding domain, sequence in italics indicate target mismatches that are homologous to the BP.

FIG. 4B. Schematic of a safety PTM in open configuration upon binding to the target.

FIG. 4C. In vitro trans-splicing reactions were carried out by incubating either safety PTM or safety PTM variants with the βHCG6 target. Splicing reactions were amplified by RT-PCR using βHCG-F and DT-3R primers; products were analyzed in a 2.0% agarose gel.

FIG. 5. Specificity of targeted trans-splicing is enhanced by the inclusion of a safety into the PTM. βHCG6 pre-mRNA (250 ng) and β-globin pre-mRNA (250 ng) were annealed together with either PTM+SF (safety) or pcPTM+Sp (linear) RNA (500 ng). In vitro trans-splicing reactions and RT-PCR analysis were performed as described under experimental procedures and the products were separated on a 2.0% agarose gel. Primers used for RT-PCR are as indicated.

FIG. 6. In the presence of increasing PTM concentration, cis-splicing is inhibited and replaced by trans-splicing. In vitro splicing reactions were performed in the presence of a constant amount of βHCG6 target pre-mRNA (100 ng) with increasing concentrations of PTM (pcPTM+Sp) RNA (52-300 ng). RT-PCR for cis-spliced and un-spliced products utilized primers βHCG-F (exon 1 specific) and βHCG-R2 (exon 2 specific—Panel A); primers βHCG-F and DT-3R were used to RT-PCR trans-spliced products (Panel B). Reaction products were analyzed on 1.5% and 2.0% agarose gels, respectively. In panel A, lane 9 represents the 60 min time point in the presence of 300 ng of PTM, which is equivalent to lane 10 in panel B.

FIG. 7A. PTMs are capable of trans-splicing in cultured human cancer cells. Total RNA was isolated from each of 4 expanded neomycin resistant H1299 lung carcinoma colonies transfected with pcSp+CRM (expressing non-toxic mutant DT-A) RT-PCR was performed using 1 μg of total RNA and 5′ biotinylated βHCG-F and non-biotinylated DT-3R primers. Single stranded DNA was purified and sequenced.

FIG. 7B. Nucleotide sequence (sense strand) (SEQ ID NO:1) of the trans-spliced product between endogenous βHCG6 target and CRM197 mutant toxin is shown (SEQ ID NO: 57). Two arrows indicate the position of the splice junction.

FIG. 8A. Schematic diagram of a double splicing pre-therapeutic mRNA.

FIG. 8B. Selective trans-splicing of a double splicing PTM. By varying the PTM concentration the PTM can be trans-spliced into either the 5′ or the 3′ splice site of the target.

FIG. 9. Schematic diagram of the use of PTM molecules for exon tagging. Two examples of PTMs are shown. The PTM on the left is capable of non-specifically trans-splicing into a target pre-mRNA 3′ splice site. The other PTM on the right is designed to non-specifically trans-splice into a target pre-mRNA 5′ splice site. A PTM mediated trans-splicing reaction will result in the production of a chimeric RNA comprising a specific tag to either the 5′ or 3′ side of an authentic exon.

FIG. 10A. Schematic diagram of constructs for use in the lacZ knock-out model. The target lacZ pre-mRNA contains the 5′ fragment of lacZ (SEQ ID NO: 58 and SEQ ID NO: 67) followed by βHCG6 intron 1 (SEQ ID NO: 59 and SEQ ID NO: 68) and the 3′ fragment of lacZ (SEQ ID NO: 60) (target 1). The PTM molecule for use in the model system was created by digesting pPTM+SP with PstI and HindIII and replacing the DT-A toxin with βHCG6 exon 2 (pc3.1PTM2).

FIG. 10B. Schematic diagram of restoration of β-Gal activity by Spliceosome Mediated RNA Trans-splicing. Schematic diagram of constructs for use in the lacZ knock-in model (pc.3.1 lacZ T2). The lacZ target pre-mRNA is identical to that target pre-mRNA used for the knock-out experiments except that it contains two stop codons (TAA TAA) in frame four codons after the 3′ splice site. The PTM molecule for use in the model system was created by digesting pPTM+SP with PstI and HindIII and replacing the DT-A toxin with functional 3′ fragment of lacZ.

FIG. 11A. Demonstration of cis- and trans-splicing when utilizing the lacZ knock-out model. The LacZ splice target 1 pre-mRNA and PTM2 were co-transfected into 293T cells. Total RNA was then isolated and analyzed by PCR for cis-spliced and trans-spliced products using the appropriate specific primers. The amplified PCR products were separated on a 2% agarose gel.

FIG. 11B-C. Assays for β-galactosidase activity. 293 cells were transfected with lacZ target 2 DNA alone (panel B) or lacZ target 2 DNA and PTM1 (panel C).

FIG. 12A. Nucleotide sequence of trans-spliced molecule demonstrating accurate trans-splicing (SEQ ID NO: 61).

FIG. 12B. Nucleotide sequences of the cis-spliced product and the trans-spliced product (SEQ ID NOS: 62, 63). The nucleotide sequences were those sequences expected for each of the different splicing reactions.

FIG. 13. Gene repair model for repair of the cystic fibrosis transmembrane regulator (CFTR) gene.

FIG. 14. RT-PCR demonstration of trans-splicing between an exogenously supplied CFTR mini-gene target and PTM. Plasmids were co-transfected into 293 embryonic kidney cells. The primers pairs used for RT-PCR reactions are listed above each lane. The lower band (471 bp) in each lane represents a trans-spliced product. The lower band in lane 1 (471 bp) was purified from a 2% Seakem agarose gel and the DNA sequence of the band was determined.

FIG. 15. DNA sequence of the trans-spliced product (lane 1, lower band shown in FIG. 14) (SEQ ID NO: 64). The DNA sequence indicates the presence of the F508 codon (CTT), exon 9 sequence is contiguous with exon 10 sequence, and the His tag sequence.

FIG. 16. Schematic representation of repair of an exogenously supplied CFTR target molecule carrying an F508 deletion in exon 10.

FIG. 17. Repair of endogenous CFTR transcripts by exon 10 replacement using a double splicing PTM. The use of a double splicing PTM permits repair of the ε508 mutation with a very short PTM molecule.

FIG. 18. Model lacZ target consisting of lacZ 5′ exon-CFTR mini-intron 9-CFTR exon 10 (delta 508)-CFTR mini-intron 10 followed by the lacZ 3′ exon. Binding domains for PTMs are bracketed.

FIG. 19. Schematic representation of double-trans-splicing PTMs designed to restore β-gal function.

FIG. 20. Schematic representation of a double-trans-splicing reaction showing the binding of DSPTM7 with DSCFT1.6 target pre-mRNA.

FIG. 21. Important structural elements of DSPTM7. The double splicing PTM has both 3′ and 5′ functional splice sites as well as binding domains.

FIG. 22. Schematic diagram of mutant double splicing PTMs (SEQ ID NO:85).

FIG. 23. Accuracy of double-trans-splicing reaction (SEQ ID NOS:86, 87).

FIG. 24. Double-trans-splicing between the target pre-mRNA and the DSPTM7 produces full-length protein. Western blot analysis of total cell lysates using polyclonal anti-β-galactosidase antiserum.

FIG. 25. Precise internal exon substitution between the DSCFT1.6 target pre-mRNA and DSPTM7 RNA by double-trans-splicing produces functionally active β-gal protein. Total cell extracts were prepared and assayed for β-gal activity using an ONPG assay.

FIG. 26. 3′ and 5′ splice sites are essential for the restoration of β-gal function by double-trans-splicing reaction.

FIG. 27. Double-trans-splicing: titration of target and PTM. Different concentrations of the target and PTM were co-transfected and analyzed for β-gal activity restoration.

FIG. 28. Constructs designed to test the specificity of double-trans-splicing reaction.

FIG. 29. Specificity of a double-trans-splicing reaction.

FIG. 30. Trans-splicing repair of the cystic fibrosis gene using a PTM that mediates a double-trans-splicing event.

FIG. 31. PTM with a long binding domain masking two splice sites and part of exon 10 in a mini-gene target (SEQ ID NO:83).

FIG. 32. Sequence of a single PCR product showing target exon 9 correctly spliced to PTM exon 10 (with modified codons) (upper panel) (SEQ ID NO:89), codon 508 in exon 10 of the PTM (middle panel)(SEQ ID NO:90) and PTM exon 10 correctly spliced to target exon 11 (lower panel)(SEQ ID NO:91). The sequence of a repaired target was generated by RT-PCR followed by PCR.

FIG. 33. Trans-splicing repair of the cystic fibrosis gene using a PTM that can perform 5′ exon replacement.

FIG. 34. Schematic diagram of three different PTM molecules with different binding domains.

FIG. 35. Schematic diagram of PTM exon 10 with modified codon usage to reduce antisense effects with its own binding domain (SEQ ID NO:92).

FIG. 36. Sequence of cis- and trans-spliced products (SEQ ID NOS:93, 94, 95, 96, 97).

FIG. 37. Model system for repair of messenger RNAs by trans-splicing. (A) Schematic illustration of a defective lacZCF9m splice target used in the present study (see Materials and Methods for details). BP, branch point; PPT, polypyrimidine tracts; ss, splice sites and pA, polyadenylation signal (SEQ ID NO:98, 99). (B) A prototype PTM showing the key components of the trans-splicing domain (SEQ ID NO:100), and the diagrams of various PTMs showing the binding domain length and approximate positions at which they bind to the target pre-mRNA. Unique restriction sites within the trans-splicing domain are N, Nhe I; S, Sac II; K, Kpn I and E, EcoR V. (C) Schematic diagram showing the binding of a PTM through antisense binding and repair of defective lacZ pre-mRNA through targeted RNA trans-splicing. Expected cis and trans-spliced products and the primer binding sites for Lac-9F, Lac-3R and Lac-5R are indicated.

FIG. 38. Efficient repair of lacZ messenger RNA. Target specific primers, Lac-9F (5′ exon) and Lac-3R (3′ exon) were used to amplify cis-spliced products (lanes 1-6), while; target and PTM specific primers, Lac-9F (5′ exon) and Lac-5R (3′ exon) were used to amplify trans-spliced products (lanes 7-15). 25-50 ng of total RNA was used to measure target cis-splicing (lanes 1-6) and 50-200 ng of total RNA was used to measure PTM induced RNA trans-splicing (lanes 7-12). Lanes 13-15, 25-50 ng of total RNA from cells transfected with lacZCF9 a control for trans-splicing. (B) Endogenous mRNA repair by trans-splicing. Lanes 1-3, RNA from cells transfected with PTM-CF14; lanes 4-6, PTM-CF22 and lanes 7-9, PTM-CF24. Lane 10, RNA from mock-transfected cells and lane 11 is a control in which reverse-transcription reaction was omitted.

FIG. 39. Messenger RNA repair leads to synthesis of full-length β-galactosidase. Lane. 1, lacZCF9 (positive control, 5 Φg); lane 2, lacZCF9m target alone (25 Φg); lane 3, PTM-CF24 alone (25 Φg) and lane 4, lacZCF9m target+PTM-CF24 (25 Φg).

FIG. 40. Messenger RNA repair by SMaRT produces functional β-galactosidase. (A) In situ detection of functional β-galactosidase produced by trans-splicing. 293T cells were either transfected (transient assay) with lacZCF9m target alone (panel A) or co-transfected with lacZCF9m target+PTM-CF24 (panel B) expression plasmids as described above. 48-hr post-transfection, cells were rinsed with PBS and stained in situ for β-gal activity. (B) Repair of a defective lacZ mRNA produces functional β-galactosidase. Target and PTM, extracts from cells transfected with either lacZCF9m target or PTM-CF24 plasmid alone, and the rest were from cells co-transfected with lacZCF9m target and one of the PTMs as indicated. (C) Endogenous mRNA repair by trans-splicing produces functional β-galactosidase. Stable cells expressing an endogenous lacZCF9m pre-mRNA target was transfected with “linear” PTMs (PTM-CF14, PTM-CF22 or PTM-CF24) as described above. Following transfection, total cell lysate was prepared and assayed for β-gal activity. The results presented are the average of two independent transfections.

FIG. 41. Messenger RNA repair is specific. (A) Experimental strategy to measure non-specific trans-splicing between lacZHCG1m pre-mRNA and “linear” PTMs. (B) Extended binding domains enhance the specificity of trans-splicing. Lanes 1-3, PTM-CF14; 4-6, PTM-CF22; 7-9, PTM-CF24; 10-12, PTM-CF26 and 13-15, PTM-CF27. (C) PTMs with very long binding domains are capable of increasing specificity. Total cell extract (5 Φl) was assayed in solution for β-gal activity and the specific activity was calculated. β-gal activity was normalized to mock and the results presented are the average of two independent transfections. Control, extract from cells transfected with lacZHCG1m target alone and the rest were co-transfected with lacZHCG1m target and one of the linear PTMs.

FIG. 42. Complete sequence of CFTR PTM 30 (5′ exon replacement PTM) showing the trans-splicing domain (underlined) (SEQ ID NO:102) and the coding sequence for exons 1-10 of the CFTR gene (SEQ ID NO:101). Modified codons in exon 10 are underlined and bold.

FIG. 43A. 153 base-pair PTM 24 Binding Domain (SEQ ID NO:103).

FIG. 43B. Complete sequence of CFTR PTM 24 (3′ exon replacement PTM) showing the trans-splicing domain (underlined) (SEQ ID NO:104) and the coding sequence for exons 10-24 of the CFTR cDNA (SEQ ID NO:105). At the end of the coding is a histidine tag and the translation stop codon.

FIG. 44A. Detailed structure of the mouse factor VII PTM containing normal mouse sequences for exons 16-26. BGH=bovine growth hormone 3′ UTR (untranslated sequence); Binding Domain=125 bp; base changes to eliminate cryptic sites are circled: F5, F6, F7, F8=primer sites.

FIG. 44B. Schematic diagram showing the extent of the binding domain in the mouse factor VIII gene.

FIG. 44C. Changes to the promoter in AAV vectors pDLZ20 and pDLZ20-M2 to eliminate cryptic donor sites in sequence upstream of the PTM binding domain (SEQ ID NOS: 108-109).

FIG. 44D. Factor VIII repair model. Schematic diagram of a PTM binding to the 3′ splice site of intron 15 of the mouse factor VIII gene.

FIG. 45. Schematic diagram of a F8 PTM with the trans-splicing domain eliminated (SEQ ID NOS:110-111). This represents a control PTM to test whether repair is a result of trans-splicing.

FIG. 46. Data indicating repair of factor VIII in Factor VIII knock out mice. Blood was assayed for factor VIII activity using a coatest assay.

FIG. 47A. Detailed structure of a mouse factor VIII PTM containing normal sequences for exons 16-26 and a C-terminal FLAG tag (SEQ ID NO:112). BGH=bovine growth hormone 3″UTR; Binding domain=125 bp.

FIG. 47B. Detailed structure of a human or canine factor VIII PTM containing normal sequences for exons 23-26 (SEQ ID NO:113).

FIG. 48. Transcription Map of HPV-16.

FIG. 49. Disruption of Human Papillomavirus Type 16 Expression by PTM. Schematic diagram of HPV-PTM 2 binding to the 3′ splice site of the HPV type 16 target pre-mRNA.

FIG. 50. E7 Targeting Strategy in which Multiple PTMs are targeted to HPV E7.

FIG. 51. PTM Design indicating the binding domain, branch point and polypyrimidine tract (SEQ ID NO:114).

FIG. 52A. HPV-PTM 1 with 80 bp binding domain targeted to 3′ ss at 409.

FIG. 52B. HPV-PTM 2 with 149 bp binding domain targeted to 3′ ss at 409.

FIG. 53. Binding Domains of HPV-PTM 3 and 4 (SEQ ID NOS:118-121).

FIG. 54. Binding Domains of HPV-PTM 5 and 6. Nucleotides in bold are modified to prevent cryptic splicing of PTMs (SEQ ID NOS:122-123).

FIG. 55. Positions of HPV-PTM targeting domains.

FIG. 56. Trans-splicing Efficiency of HPV-PTMs in 293 T Cells. 293T cells were con-transfected with 2 μg of p1059 target and 1.5 μg of PTM expression plasmids. 48 hr post-transfection, total RNA was isolated and analyzed by RT-PCR. Target specific primers, oJMD15 and JMD16 were used to amplify cis-spliced products (lanes 1-11, upper panel), while; target and PTM specific primers, oJMD15 and Lac-6R were used to amplify transspliced products (lanes 1-12, lower panel). Lanes 13-14 (upper panel), RNA isolated from cells that are transfected with lacZCF9 and HPV-PTM1 and 2 respectively, hence, serve as controls for evaluating the specificity of HPV-PTMs.

FIG. 57. Nucleotide sequence showing the trans-splice junctions between the HPV target pre-mRNA and the PTM. The RT-PCR product was purified and sequenced directly using primer Lac5R (binds to 3′ exon of the PTM) (SEQ ID NO:124). The arrow indicate trans-splice junction between E6 of HPV pre-mRNA target and lacZ 3′ exon of the PTM.

FIG. 58. Trans-splicing in 293 cells (Co-transfections) Quantification of trans-splicing efficiency was determined using real-time QRT-PCR.

FIG. 59. Trans-splicing efficiency of HPV-PTMs into an endogenous pre-mRNA target. SiHa and CaSki cells were transfected wit 1.5 μg of either HPV-PTM1, 2 or CFTR targets PTM14 or 27 expression plasmids. 48 hr post-transplicing, total RNA was isolated and analyzed by RT-PCR. Trans-splicing between the endogenous HPV target and the PTm was detected using target and PTM specific primers oJMD15 and Lac-16R. The expected trans-spliced product (418 bp) is clearly visible in cells that are transfected with HPV-PTMs (lanes 2-3 and 5-7) but not in control (lanes 1 and 4). In addition, trans-splicing is also detected in lane 8 due to non-specific trans-splicing.

FIG. 60. Accurate Trans-splicing of HPV-PTM1 in SiHa Cells. Target pre-mRNA was endogenous mRNA (SEQ ID NO:125). Sequence analysis of trans-spliced chimeric RNA indicates that trans-splicing is accurate.

FIG. 61. Quantification of trans-splicing efficiency in SiHa cells using real-time QRT-PCR.

FIG. 62. Trans-splicing efficiency of HPV-PTM 1, HPV-PTM 5, & HPV-PTM 6 in SiHa cells. Analysis of total RNA was performed using RT-PCR.

FIG. 63. Deletion of polypyrimidine tract abolishes trans-splicing. Lanes 1 and 2 represent RNA from cells transfected with mutant HPV-PPT. Lanes 3 and 4 represent RNA from cells transfected with HPV-PTM5 plasmid. 269 bp product resulting from trans-splicing is detected.

FIG. 64. Schematic Diagram of a PTM binding to the 5′ splice site of the HPV mini-gene target and the resulting trans-spliced chimera RNA.

FIG. 65. Double Trans-splicing. Schematic diagram of a double trans-splicing PTM binding to the 3′ and 5′ splice sites of the HPV mini-gene target. The resultant trans-spliced mRNA is shown.

FIG. 66A. Trans-splicing by 3′ exon replacement. Schematic diagram of a PTM binding to the 3′ splice site of the HPV mini-gene target.

FIG. 66B. Trans-splicing by 5′ exon replacement. Schematic diagram of a PTM binding to the 5′ splice site of the HPV mini-gene target.

FIG. 67. Schematic of a double splicing HPV-PTM designed for internal exon replacement.

### DETAILED DESCRIPTION OF THE INVENTION

The present invention relates to compositions comprising pre-trans-splicing molecules (PTMs) and the use of such molecules for generating novel nucleic acid molecules. The PTMs of the invention comprise one or more target binding domains that are designed to specifically bind to pre-mRNA, a 3′ splice region that includes a branch point, pyrimidine tract and a 3′ splice acceptor site and/or a 5′ splice donor site; and one or more spacer regions that separate the RNA splice site from the target binding domain. In addition, the PTMs of the invention can be engineered to contain any nucleotide sequences such as those encoding a translatable protein product.

The methods of the invention encompass contacting the PTMs of the invention with a natural pre-mRNA under conditions in which a portion of the PTM is trans-spliced to a portion of the natural pre-mRNA to form a novel chimeric RNA. The target pre-mRNA is chosen as a target due to its expression within a specific cell type thus providing a mechanism for targeting expression of a novel RNA to a selected cell type. The resulting chimeric RNA may provide a desired function, or may produce a gene product in the specific cell type. The specific cells may include, but are not limited to those infected with viral or other infectious agents, benign or malignant neoplasms, or components of the immune system which are involved in autoimmune disease or tissue rejection. Specificity is achieved by modification of the binding domain of the PTM to bind to the target endogenous pre-mRNA. The gene products encoded by the chimeric RNA can be any gene, including genes having clinical usefulness, for example, therapeutic or marker genes, and genes encoding toxins:

Structure of the Pre-Trans-Splicing Molecules

The present invention provides compositions for use in generating novel chimeric nucleic acid molecules through targeted trans-splicing. The PTMs of the invention comprise (i) one or more target binding domains that targets binding of the PTM to a pre-mRNA (ii) a 3′ splice region that includes a branch point, pyrimidine tract and a 3′ splice acceptor site and/or 5′ splice donor site; and (iii) one or more spacer regions to separate the RNA splice site from the target binding domain. Additionally, the PTMs can be engineered to contain any nucleotide sequence encoding a translatable protein product. In yet another embodiment of the invention, the PTMs can be engineered to contain nucleotide sequences that inhibit the translation of the chimeric RNA molecule. For example, the nucleotide sequences may contain translational stop codons or nucleotide sequences that form secondary structures and thereby inhibit translation. Alternatively, the chimeric RNA may function as an antisense molecule thereby inhibiting translation of the RNA to which it binds.

The target binding domain of the PTM may contain multiple binding domains which are complementary to and in anti-sense orientation to the targeted region of the selected pre-mRNA. As used herein, a target binding domain is defined as any sequence that confers specificity of binding and anchors the pre-mRNA closely in space so that the spliceosome processing machinery of the nucleus can trans-splice a portion of the PTM to a portion of the pre-mRNA. The target binding domains may comprise up to several thousand nucleotides. In preferred embodiments of the invention the binding domains may comprise at least 10 to 30 and up to several hundred nucleotides. As demonstrated herein, the specificity of the PTM can be increased significantly by increasing the length of the target binding domain. For example, the target binding domain may comprise several hundred nucleotides or more. In addition, although the target binding domain may be “linear” it is understood that the RNA may fold to form secondary structures that may stabilize the complex thereby increasing the efficiency of splicing. A second target binding region may be placed at the 3′ end of the molecule and can be incorporated into the PTM of the invention. Absolute complementarity, although preferred, is not required. A sequence “complementary” to a portion of an RNA, as referred to herein, means a sequence having sufficient complementarity to be able to hybridize with the RNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

Where the PTMs are designed for use in intron-exon tagging or for peptide affinity tagging, a library of PTMs is genetically engineered to contain random nucleotide sequences in the target binding domain. Alternatively, for intron-exon tagging the PTMs may be genetically engineered so as to lack target binding domains. The goal of generating such a library of PTM molecules is that the library will contain a population of PTM molecules capable of binding to each RNA molecule expressed in the cell. A recombinant expression vector can be genetically engineered to contain a coding region for a PTM including a restriction endonuclease site that can be used for insertion of random DNA fragments into the PTM to form random target binding domains. The random nucleotide sequences to be included in the PTM as target binding domains can be generated using a variety of different methods well known to those of skill in the art, including but not limited to, partial digestion of DNA with restriction enzymes or mechanical shearing of DNA to generate random fragments of DNA. Random binding domain regions may also be generated by degenerate oligonucleotide synthesis. The degenerate oligonucleotides can be engineered to have restriction endonuclease recognition sites on each end to facilitate cloning into a PTM molecule for production of a library of PTM molecules having degenerate binding domains.

Binding may also be achieved through other mechanisms, for example, through triple helix formation or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

The PTM molecule also contains a 3′ splice region that includes a branch point, pyrimidine tract and a 3′ splice acceptor AG site and/or a 5′ splice donor site. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine). The underlined A is the site of branch formation. A polypyrimidine tract is located between the branch point and the splice site acceptor and is important for different branch point utilization and 3′ splice site recognition.

Further, PTMs comprising a 3′ acceptor site (AG) may be genetically engineered. Such PTMs may further comprise a pyrimidine tract and/or branch point sequence.

Recently, pre-messenger RNA introns beginning with the dinucleotide AU and ending with the dinucleotide AC have been identified and referred to as U12 introns. U12 intron sequences as well as any sequences that function as splice acceptor/donor sequences may also be used in PTMs.

A spacer region to separate the RNA splice site from the target binding domain is also included in the PTM. The spacer region can have features such as stop codons which would block any translation of an unspliced PTM and/or sequences that enhance trans-splicing to the target pre-mRNA.

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding/targeting portion(s) of the PTM, the 3′ and/or 5′splice site is uncovered and becomes fully active.

The “safety” consists of one or more complementary stretches of cis-sequence (or could be a second, separate, strand of nucleic acid) which weakly binds to one or both sides of the PTM branch point, pyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

A nucleotide sequence encoding a translatable protein capable of producing an effect, such as cell death, or alternatively, one that restores a missing function or acts as a marker, is included in the PTM of the invention. For example, the nucleotide sequence can include those sequences encoding gene products missing or altered in known genetic diseases. Alternatively, the nucleotide sequences can encode marker proteins or peptides which may be used to identify or image cells. In yet another embodiment of the invention nucleotide sequences encoding affinity tags such as, HIS tags (6 consecutive histidine residues) (Janknecht, et al., 1991, Proc. Natl. Acad. Sci. USA 88:8972-8976), the C-terminus of glutathione-S-transferase (GST) (Smith and Johnson, 1986, Proc. Natl. Acad. Sci. USA 83:8703-8707) (Pharmacia) or FLAG (Asp-Tyr-Lys-Asp-Asp-Asp-Lys) (SEQ ID NO: 66) (Eastman Kodak/IBI, Rochester, N.Y.) can be included in PTM molecules for use in affinity purification. The use of PTMs containing such nucleotide sequences results in the production of a chimeric RNA encoding a fusion protein containing peptide sequences normally expressed in a cell linked to the peptide affinity tag. The affinity tag provides a method for the rapid purification and identification of peptide sequences expressed in the cell. In a preferred embodiment the nucleotide sequences may encode toxins or other proteins which provide some function which enhances the susceptibility of the cells to subsequent treatments, such as radiation or chemotherapy.

In a highly preferred embodiment of the invention a PTM molecule is designed to contain nucleotide sequences encoding the Diphtheria toxin subunit A (Greenfield, L., et al., 1983, Proc. Nat'l. Acad. Sci. USA 80: 6853-6857). Diphtheria toxin subunit A contains enzymatic toxin activity and will function if expressed or delivered into human cells resulting in cell death. Furthermore, various other known peptide toxins may be used in the present invention, including but not limited to, ricin, Pseudomonus toxin, Shiga toxin and exotoxin A.

Additional features can be added to the PTM molecule either after, or before, the nucleotide sequence encoding a translatable protein, such as polyadenylation signals or 5′ splice sequences to enhance splicing, additional binding regions, “safety”-self complementary regions, additional splice sites, or protective groups to modulate the stability of the molecule and prevent degradation.

Additional features that may be incorporated into the PTMs of the invention include stop codons or other elements in the region between the binding domain and the splice site to prevent unspliced pre-mRNA expression. In another embodiment of the invention, PTMs can be generated with a second anti-sense binding domain downstream from the nucleotide sequences encoding a translatable protein to promote binding to the 3′ target intron or exon and to block the fixed authentic cis-5′ splice site (U5 and/or U1 binding sites).

PTMs may also be generated that require a double-trans-splicing reaction for generation of a chimeric trans-spliced product. Such PTMs could be used to replace an internal exon which could be used for RNA repair. PTMs designed to promote two trans-splicing reactions are engineered as described above, however, they contain both 5′ donor sites and 3′ splice acceptor sites. In addition, the PTMs may comprise two or more binding domains and splicer regions. The splicer regions may be place between the multiple binding domains and splice sites or alternatively between the multiple binding domains.

Further elements such as a 3′ hairpin structure, circularized RNA, nucleotide base modification, or a synthetic analog can be incorporated into PTMs to promote or facilitate nuclear localization and spliceosomal incorporation, and intra-cellular stability.

Additionally, when engineering PTMs for use in plant cells it may not be necessary to include conserved branch point sequences or polypyrimidine tracts as these sequences may not be essential for intron processing in plants. However, a 3′ splice acceptor site and/or 5′ splice donor site, such as those required for splicing in vertebrates and yeast, will be included. Further, the efficiency of splicing in plants may be increased by also including UA-rich intronic sequences. The skilled artisan will recognize that any sequences that are capable of mediating a trans-splicing reaction in plants may be used.

The PTMs of the invention can be used in methods designed to produce a novel chimeric RNA in a target cell. The methods of the present invention comprise delivering to the target cell a PTM which may be in any form used by one skilled in the art, for example, an RNA molecule, or a DNA vector which is transcribed into a RNA molecule, wherein said PTM binds to a pre-mRNA and mediates a trans-splicing reaction resulting in formation of a chimeric RNA comprising a portion of the PTM molecule spliced to a portion of the pre-mRNA.

Synthesis of the Trans-Splicing Molecules

The nucleic acid molecules of the invention can be RNA or DNA or derivatives or modified versions thereof, single-stranded or double-stranded. By nucleic acid is meant a PTM molecule or a nucleic acid molecule encoding a PTM molecule, whether composed of deoxyribonucleotides or ribonucleosides, and whether composed of phosphodiester linkages or modified linkages. The term nucleic acid also specifically includes nucleic acids composed of bases other than the five biologically occurring bases (adenine, guanine, thymine, cytosine and uracil).

The RNA and DNA molecules of the invention can be prepared by any method known in the art for the synthesis of DNA and RNA molecules. For example, the nucleic acids may be chemically synthesized using commercially available reagents and synthesizers by methods that are well known in the art (see, e.e., Gait, 1985, Oligonucleotide Synthesis: A Practical Approach, IRL Press, Oxford, England). Alternatively, RNA molecules can be generated by in vitro and in vivo transcription of DNA sequences encoding the RNA molecule. Such DNA sequences can be incorporated into a wide variety of vectors which incorporate suitable RNA polymerase promoters such as the T7 or SP6 polymerase promoters. RNAs may be produced in high yield via in vitro transcription using plasmids such as SPS65 (Promega Corporation, Madison, Wis.). In addition, RNA amplification methods such as Q-β amplification can be utilized to produce RNAs.

The nucleic acid molecules can be modified at the base moiety, sugar moiety, or phosphate backbone, for example, to improve stability of the molecule, hybridization, transport into the cell, etc. For example, modification of a PTM to reduce the overall charge can enhance the cellular uptake of the molecule. In addition modifications can be made to reduce susceptibility to nuclease degradation. The nucleic acid molecules may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc. Various other well-known modifications to the nucleic acid molecules can be introduced as a means of increasing intracellular stability and half-life. Possible modifications include, but are not limited to, the addition of flanking sequences of ribo- or deoxy-nucleotides to the 5′ and/or 3′ ends of the molecule. In some circumstances where increased stability is desired, nucleic acids having modified internucleoside linkages such as 2′-0-methylation may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic acids can be purified by reverse phase chromatography or gel electrophoresis. Of course, the skilled artisan will recognize that the method of purification will depend in part on the size of the nucleic acid to be purified.

In instances where a nucleic acid molecule encoding a PTM is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target cells in the patient will result in the transcription of sufficient amounts of PTMs that will form complementary base pairs with the endogenously expressed pre-mRNA targets and thereby facilitate a trans-splicing reaction between the complexed nucleic acid molecules. For example, a vector can be introduced in vivo such that it is taken up by a cell and directs the transcription of the PTM molecule. Such a vector can remain episomal or become chromosomally integrated, as long as it can be transcribed to produce the desired RNA. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter known in the art to act in mammalian, preferably human cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:14411445), the regulatory sequences of the metallothionein gene (Brinster et al., 1982, Nature 296:39-42), the viral CMV promoter, the human chorionic gonadotropin-β promoter (Hollenberg et al., 1994, Mol. Cell. Endocrinology 106:111-119), etc. Any type of plasmid, cosmid, YAC or viral vector can be used to prepare the recombinant DNA construct which can be introduced directly into the tissue site. Alternatively, viral vectors can be used which selectively infect the desired target cell.

For use of PTMs encoding peptide affinity purification tags, it is desirable to insert nucleotide sequences containing random target binding sites into the PTMs and clone them into a selectable mammalian expression vector system. A number of selection systems can be used, including but not limited to selection for expression of the herpes simplex virus thymidine kinase, hypoxanthine-guanine phosphoribosyltransterase and adenine phosphoribosyl transferase protein in tk-, hgprt- or aprt-deficient cells, respectively. Also, anti-metabolic resistance can be used as the basis of selection for dihydrofolate tranferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin. In a preferred embodiment of the invention, the cell culture is transformed at a low ratio of vector to cell such that there will be only a single vector, or a limited number of vectors, present in any one cell. Vectors for use in the practice of the invention include any eukaryotic expression vectors, including but not limited to viral expression vectors such as those derived from the class of retroviruses or adeno-associated viruses.

Uses and Administration of Trans-Splicing Molecules

Use of PTM Molecules for Gene Regulation, Gene Repair and Targeted Cell Death

The compositions and methods of the present invention will have a variety of different applications including gene regulation, gene repair and targeted cell death. For example, trans-splicing can be used to introduce a protein with toxic properties into a cell. In addition, PTMs can be engineered to bind to viral mRNA and destroy the function of the viral mRNA, or alternatively, to destroy any cell expressing the viral mRNA. In yet another embodiment of the invention, PTMs can be engineered to place a stop codon in a deleterious mRNA transcript thereby decreasing the expression of that transcript.

In an embodiment of the invention PTM molecules were designed to bind to papilloma virus RNA and inhibit the function of the viral RNA. Specifically anti-HPV PTMs were designed to specifically target HPV pre-mRNAs and result in the expression of a disruptive or toxic protein only in the HPV-infected cancer cells. Thus, the invention provides PTM molecules designed to inhibit the function of papilloma virus RNA. Such papilloma viruses, include but are not limited to mammalian papillomaviruses including human papillomaviruses.

The papilloma viruses are a group of small DNA viruses which induce papillomas (warts) in a variety of vertebrates, including human. In addition, human papilloma virus is one of the most common causes of sexually transmitted diseases in the country and the vast majority of cervical cancers are associated with oncogenic human papillomaviruses and express viral mRNAs encoding the E6 and E7 oncoproteins. Thus, the PTM molecules of the invention may be used to inhibit the proliferation of papillomaviruses within an infected host.

Targeted trans-splicing, including double-trans-splicing reactions, 3′ exon replacement and/or 5′ exon replacement can be used to repair or correct transcripts that are either truncated or contain point mutations. The PTMs of the invention are designed to cleave a targeted transcript upstream or downstream of a specific mutation or upstream of a premature 3′ and correct the mutant transcript via a trans-splicing reaction which replaces the portion of the transcript containing the mutation with a functional sequence.

In addition, double trans-splicing reactions may be used for the selective expression of a toxin in tumor cells. For example, PTMs can be designed to replace the second exon of the human β-chronic gonadotropin-6 (βhCG6) gene transcripts and to deliver an exon encoding the subunit A of diptheria toxin (DT-A). Expression of DT-A in the absence of subunit B should lead to toxicity only in the cells expressing the gene. βhCG6 is a prototypical target for genetic modification by trans-splicing. The sequence and the structure of the βhCG6 gene are completely known and the pattern of splicing has been determined. The βhCG6 gene is highly expressed in many types of solid tumors, including many non-germ line tumors, but the βhCG6 gene is silent in the majority cells in a normal adult. Therefore, the βhCG6 pre-mRNA represents a desirable target for a trans-splicing reaction designed to produce tumor-specific toxicity.

The first exon of βhCG6 pre-mRNA is ideal in that it encodes only five amino acids, including the initiator AUG, which should result in minimal interference with the proper folding of the DT-A toxin while providing the required signals for effective translation of the trans-spliced mRNA. The DT-A exon, which is designed to include a stop codon to prevent chimeric protein formation, will be engineered to trans-splice into the last exon of the βhCG6 gene. The last exon of the βhCG6 gene provides the construct with the appropriate signals to polyadenylate the mRNA and ensure translation.

Cystic fibrosis (CF) is one of the most common fatal genetic disease in humans. Based on both genetic and molecular analyses, the gene associated with cystic fibrosis has been isolated and its protein product deduced (Kerem, B. S. et al., 1989, Science 245:1073-1080; Riordan et al., 1989, Science 245:1066-1073; Rommans, et al., 1989, Science 245:1059-1065). The protein product of the CF associated gene is called the cystic fibrosis transmembrane conductance regulator (CFTR). In a specific embodiment of the invention, a trans-splicing reaction will be used to correct a genetic defect in the DNA sequence encoding the cystic fibrosis transmembrane regulator (CFTR) whereby the DNA sequence encoding the cystic fibrosis trans-membrane regulator protein is expressed and a functional chloride ion channel is produced in the airway epithelial cells of a patient.

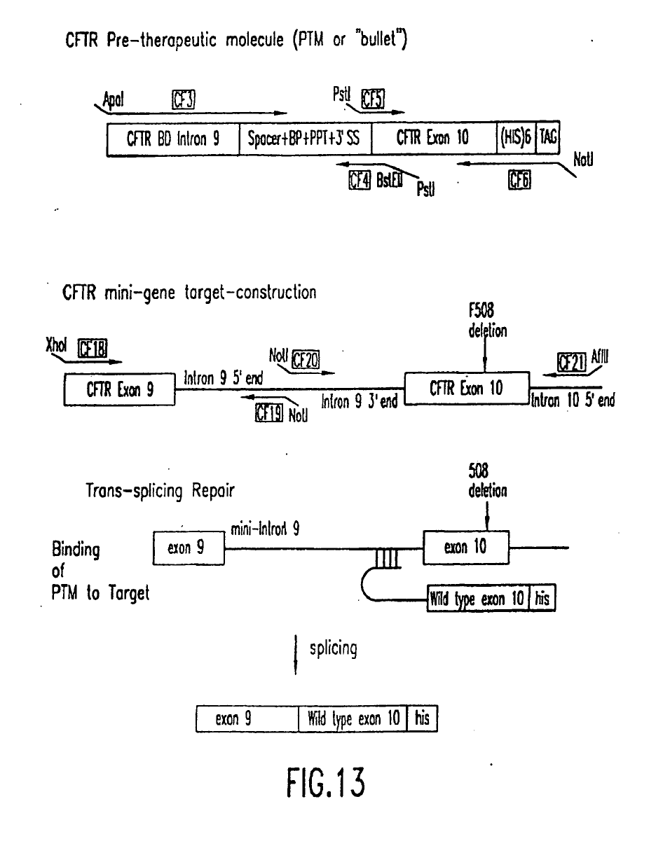

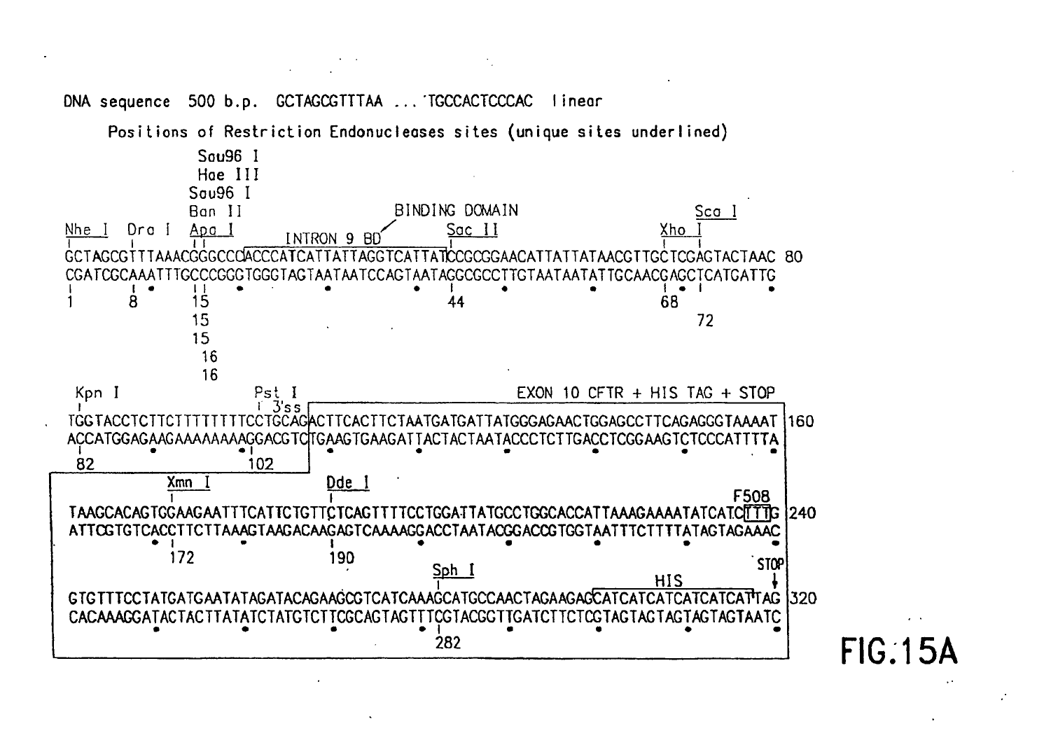

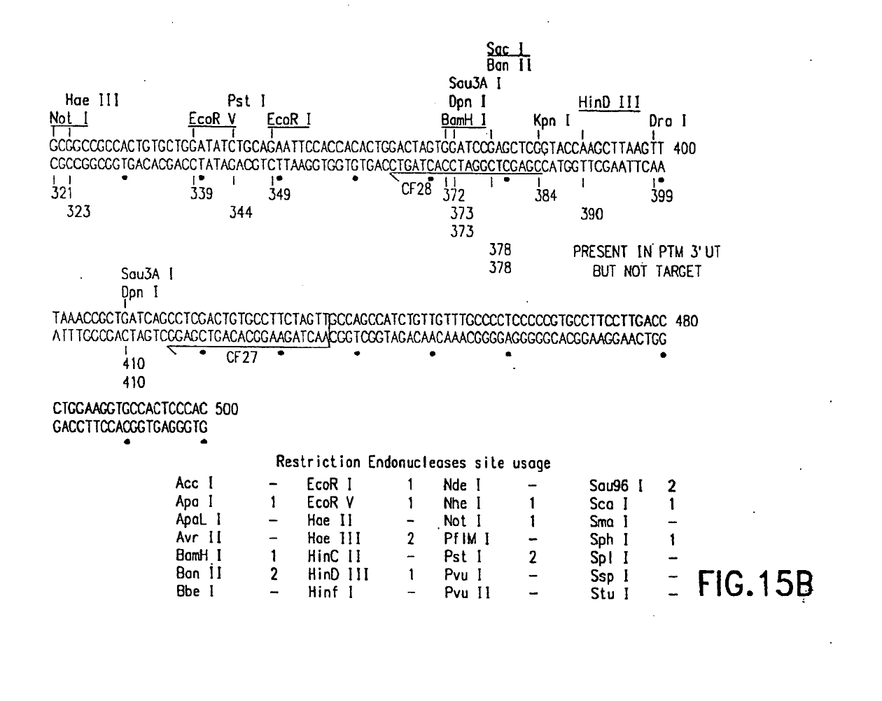

Population studies have indicated that the most common cystic fibrosis mutation is a deletion of the three nucleotides in exon 10 that encode phenylalanine at position 508 of the CFTR amino acid sequence. As indicated in FIG. 15, a trans-splicing reaction was capable of correcting the deletion at position 508 in the CFTR amino acid sequence. The PTM used for correction of the genetic defect contained a CFTR BD intron 9 sequence, a spacer sequence, a branch point, a polypyrimidine tract, a 3′ splice site and a wild type CFTR BD exon 10 sequence (FIG. 13). The successful correction of the mutated DNA encoding CFTR utilizing a trans-splicing reaction supports the general application of PTMs for correction of genetic defects.

HemophiliaA is an X-linked bleeding disorder characterized by a deficiency in the activity of factor VIII, a n important component of the coagulation cascade. The incidence of hemophilia A is approximately 1 in 5,000 to 10,000 males. Affected individuals suffer joint and muscle hemorrhage, easy bruising, and prolonged bleeding from wounds. Hemophilia A arises from a variety of mutations within the factor VIII gene. The gene comprises 26 exons and spans 186 kb. About 95 percent of those patients with hemophilia A in whom mutations have been characterized, have point mutations in the gene. In a specific embodiment of the invention, a trans-splicing reaction will be used to correct a genetic defect in the DNA sequence encoding factor VIII whereby the DNA sequence encoding the factor VIII protein is expressed and a functional clotting factor is produced in the plasma of a patient. The PTMs of the invention can be genetically engineered to repair any exon of interest, or combination of exons for the purpose of correcting a defect in the coding region of the factor VIII gene.

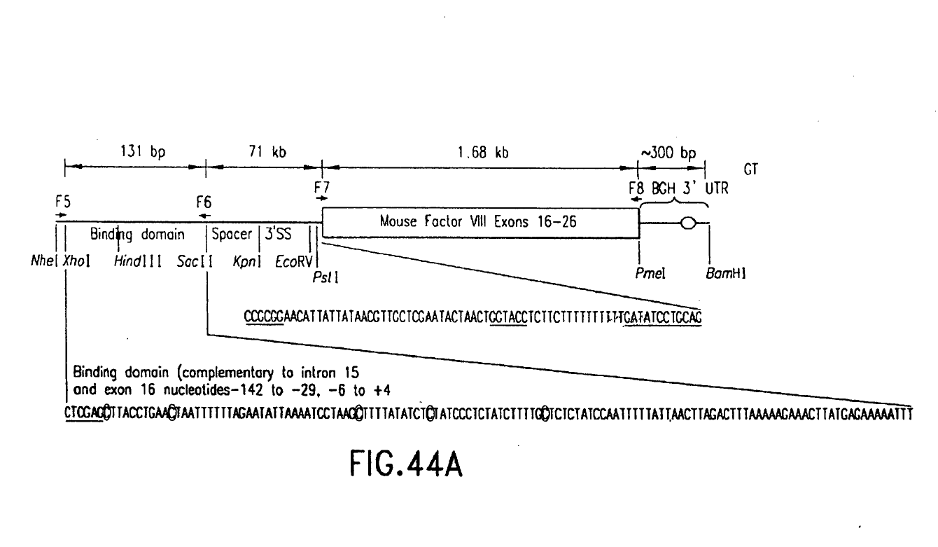

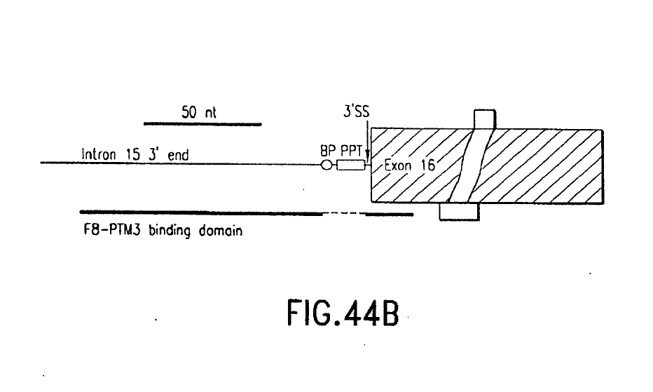

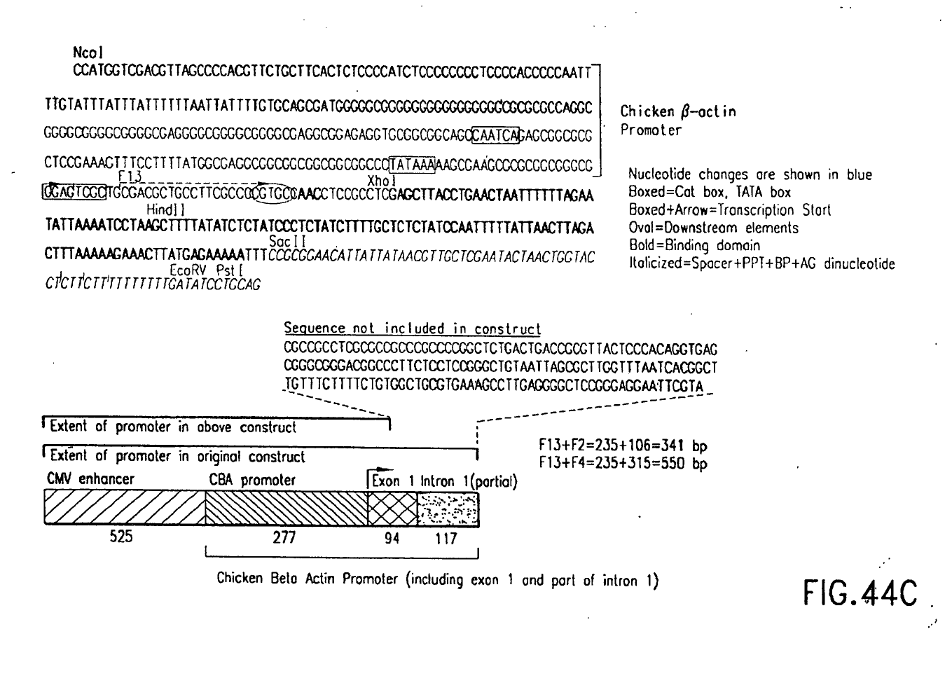

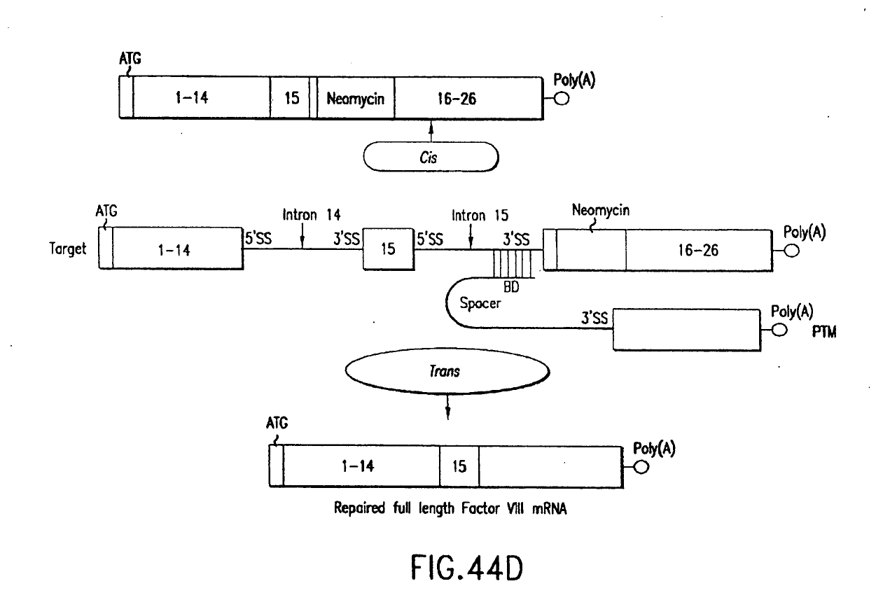

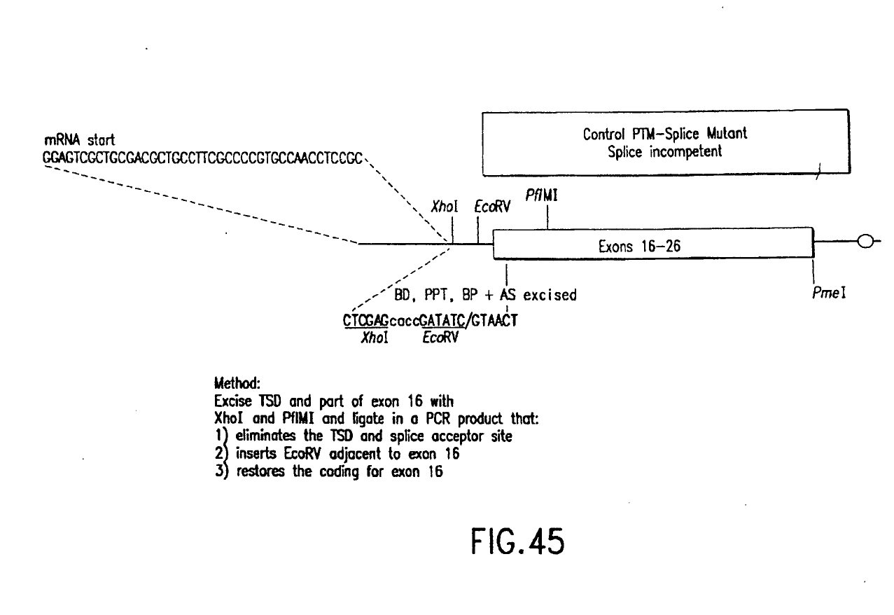

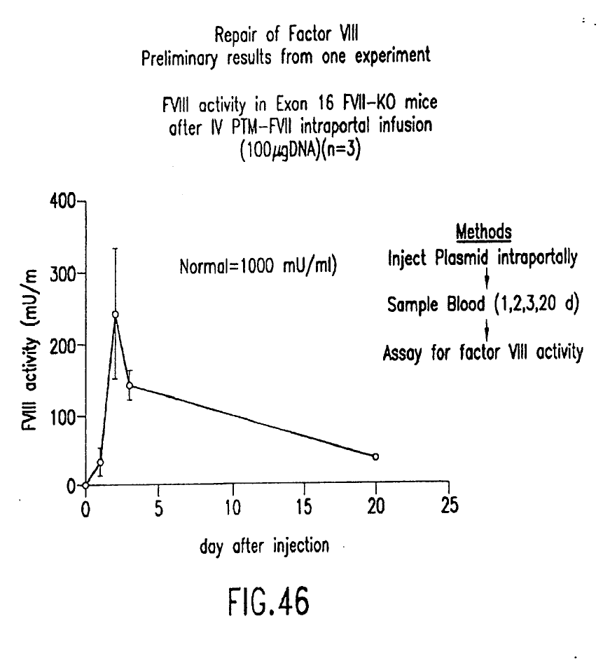

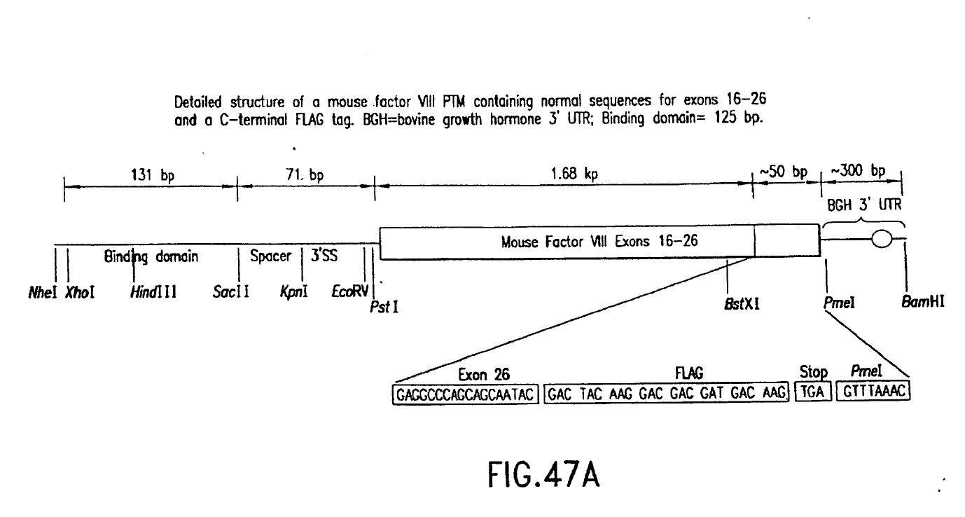

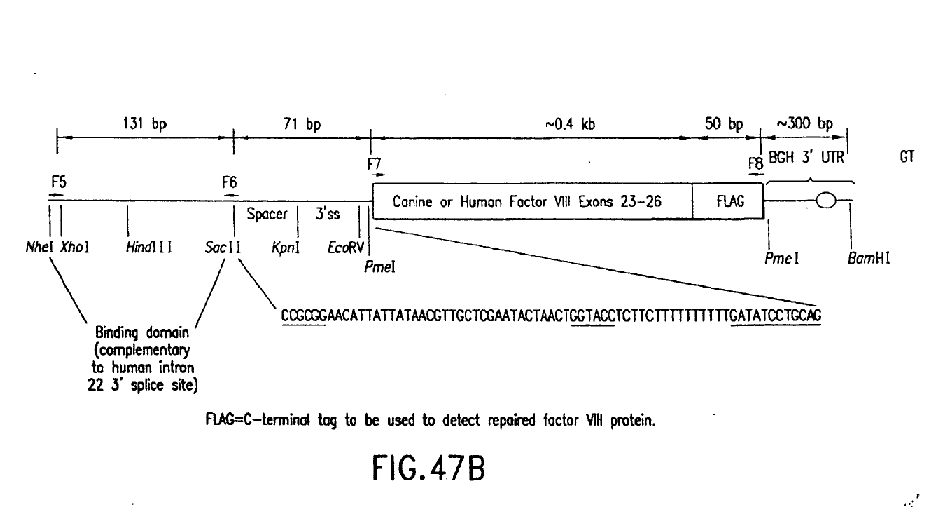

Genetic studies have indicated that the most common factor VIII mutation(s) are be generated. As indicated in FIG. 46, a trans-splicing reaction was capable of correcting the mutation in the factor VIII amino acid sequence. The mutation was created by an insertion of the neomycin gene into exon 16 and intron 16 of the mouse gene, interrupting the open reading frame of exon 16 and eliminating intron 16's 3′ splice donor site. The PTM used for correction of the genetic defect contained factor VIII exons 16-24 coding sequences, a spacer sequence, a branch point, a polypyrimidine tract, and a 3′ acceptor splice (FIG. 44A). The successful correction of the mutated DNA encoding factor VIII utilizing a trans-splicing reaction further supports the general application of PTMs for correction of genetic defects.

The methods and compositions of the invention may also be used to regulate gene expression in plants. For example, trans-splicing may be used to place the expression of any engineered gene under the natural regulation of a chosen target plant gene, thereby regulating the expression of the engineered gene. Trans-splicing may also be used to prevent the expression of engineered genes in non-host plants or to convert an endogenous gene product into a more desirable product.

In a specific embodiment of the invention tran-splicing may be used to regulate the expression of the insecticidal gene that produces Bt toxin (Bacillus thuringiensis). For example, the PTM may be designed to trans-splice into an injury response gene (pre-mRNA) that is expressed only after an insect bites the plant. Thus, all cells of the plant would carry the gene for Bt in the PTM, but the cells would only produce Bt when and where an insect injures the plant. The rest of the plant will make little or no Bt. A PTM could trans-splice the Bt gene into any chosen gene with a desired pattern of expression. Further, it should be possible to target a PTM so that no Bt is produced in the edible portion of the plant.

One advantage associated with the use of PTMs is that the PTM acquires the native gene control elements of the target gene, thus, reducing the time and effort that might otherwise be spent attempting to identify and reconstitute appropriate regulatory sequences upstream of an engineered gene. Thus, expression of the PTM regulated gene should occur only in those plant cells containing the target pre-mRNA. By targeting a gene not expressed in the edible portion of the plant or in the pollen, trans-splicing can alleviate opposition to genetically modified plants, as consumers would not be eating the proteins made from modified genes. The edible portion of such crops should test negative for genetically modified proteins.

In addition, PTM can be targeted to a unique sequence of the host gene that is not present in other plants. Therefore, even if the gene (DNA) which encodes the PTM jumps to another species of plant, the PTM gene will not have an appropriate target for trans-splicing. Thus, trans-splicing offers a “fail-safe” mode for prevention of gene “jumping” to other plant species: the PTM gene will be expressed only in the engineered host plant, which contains the appropriate target pre-mRNA. Expression in non-engineered plants would not be possible.

Trans-splicing also provides a more efficient way to convert one gene product into another. For example, trans-splicing ribozymes and chimeric oligos can be incorporated into corn genomes to modify the ratio of saturated to unsaturated oils. Trans-splicing can also be used to convert one gene product into another.

Various delivery systems are known and can be used to transfer the compositions of the invention into cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral or other vector, injection of DNA, electroporation, calcium phosphate mediated transfection, etc.

The compositions and methods can be used to treat cancer and other serious viral infections, autoimmune disorders, and other pathological conditions in which the alteration or elimination of a specific cell type would be beneficial. Additionally, the compositions and methods may also be used to provide a gene encoding a functional biologically active molecule to cells of an individual with an inherited genetic disorder where expression of the missing or mutant gene product produces a normal phenotype.

In a preferred embodiment, nucleic acids comprising a sequence encoding a PTM are administered to promote PTM function, by way of gene delivery and expression into a host cell. In this embodiment of the invention, the nucleic acid mediates an effect by promoting PTM production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the nucleic acid into a host cell may be either direct, in which case the host is directly exposed to the nucleic acid or nucleic acid-carrying vector, or indirect, in which case, host cells are first transformed with the nucleic acid in vitro, then transplanted into the host. These two approaches are known, respectively, as in vivo or ex vivo gene delivery.

In a specific embodiment, the nucleic acid is directly administered in vivo, where it is expressed to produce the PTM. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

The present invention also provides for pharmaceutical compositions comprising an effective amount of a PTM or a nucleic acid encoding a PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

In specific embodiments, pharmaceutical compositions are administered: (1) in diseases or disorders involving an absence or decreased (relative to normal or desired) level of an endogenous protein or function, for example, in hosts where the protein is lacking, genetically defective, biologically inactive or underactive, or under expressed; or (2) in diseases or disorders wherein, in vitro or in vivo, assays indicate the utility of PTMs that inhibit the function of a particular protein. The activity of the protein encoded for by the chimeric mRNA resulting from the PTM mediated trans-splicing reaction can be readily detected, e.g., by obtaining a host tissue sample (e.g., from biopsy tissue) and assaying it in vitro for mRNA or protein levels, structure and/or activity of the expressed chimeric mRNA. Many methods standard in the art can be thus employed, including but not limited to immunoassays to detect and/or visualize the protein encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA expression by detecting and/or visualizing the presence of chimeric mRNA (e.g., Northern assays, dot blots, in situ hybridization, and Reverse-Transcription PCR, etc.), etc.

The present invention also provides for pharmaceutical compositions comprising an effective amount of a PTM or a nucleic acid encoding a PTM, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin. In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Other control release drug delivery systems, such as nanoparticles, matrices such as controlled-release polymers, hydrogels.

The PTM will be administered in amounts which are effective to produce the desired effect in the targeted cell. Effective dosages of the PTMs can be determined through procedures well known to those in the art which address such parameters as biological half-life, bioavailability and toxicity. The amount of the composition of the invention which will be effective will depend on the nature of the disease or disorder being treated, and can be determined by standard clinical techniques. In addition, in vitro assays may optionally be employed to help identify optimal dosage ranges.

The present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human administration.

Use of PTM Molecules for Exon Tagging

In view of current efforts to sequence and characterize the genomes of humans and other organisms, there is a need for methods that facilitate such characterization. A majority of the information currently obtained by genomic mapping and sequencing is derived from complementary DNA (cDNA) libraries, which are made by reverse transcription of mRNA into cDNA. Unfortunately, this process causes the loss of information concerning intron sequences and the location of exon/intron boundaries.

The present invention encompasses a method for mapping exon-intron boundaries in pre-mRNA molecules comprising (i) contacting a pre-trans-splicing molecule with a pre-mRNA molecule under conditions in which a portion of the pre-trans-splicing molecule is trans-spliced to a portion of the target pre-mRNA to form a chimeric mRNA; (ii) amplifying the chimeric mRNA molecule; (iii) selectively purifying the amplified molecule; and (iv) determining the nucleotide sequence of the amplified molecule thereby identifying the intron-exon boundaries.

In an embodiment of the present invention, PTMs can be used in trans-splicing reactions to locate exon-intron boundaries in pre-mRNAs molecules. PTMs for use in mapping of intron-exon boundaries have structures similar to those described above in Section 5.1. Specifically, the PTMs contain (i) a target binding domain that is designed to bind to many pre-mRNAs: (ii) a 3′ splice region that includes a branch point, pyrimidine tract and a 3′ splice acceptor site, or a 5′ splice donor site; (iii) a spacer region that separates the mRNA splice site from the target binding domain; and (iv) a tag region that will be trans-spliced onto a pre-mRNA. Alternatively, the PTMs to be used to locate exon-intron boundaries may be engineered to contain no target binding domain.

For purposes of intron-exon mapping, the PTMs are genetically engineered to contain target binding domains comprising random nucleotide sequences. The random nucleotide sequences contain at least 15-30 and up to several hundred nucleotide sequences capable of binding and anchoring a pre-mRNA so that the spliceosome processing machinery of the nucleus can trans-splice a portion (tag or marker region) of the PTM to a portion of the pre-mRNA. PTMs containing short target binding domains, or containing inosines bind under less stringent conditions to the pre-mRNA molecules. In addition, strong branch point sequences and pyrimidine tracts serve to increase the non-specificity of PTM trans-splicing.

The random nucleotide sequences used as target binding domains in the PTM molecules can be generated using a variety of different methods, including, but not limited to, partial digestion of DNA with restriction endonucleases or mechanical shearing of the DNA. The use of such random nucleotide sequences is designed to generate a vast array of PTM molecules with different binding activities for each target pre-mRNA expressed in a cell. Randomized libraries of oligonucleotides can be synthesized with appropriate restriction endonucleases recognition sites on each end for cloning into PTM molecules genetically engineered into plasmid vectors. When the randomized oligonucleotides are litigated and expressed, a randomized binding library of PTMs is generated.

In a specific embodiment of the invention, an expression library encoding PTM molecules containing target binding domains comprising random nucleotide sequences can be generated using a variety of methods which are well known to those of skill in the art. Ideally, the library is complex enough to contain PTM molecules capable of interacting with each target pre-mRNA expressed in a cell.

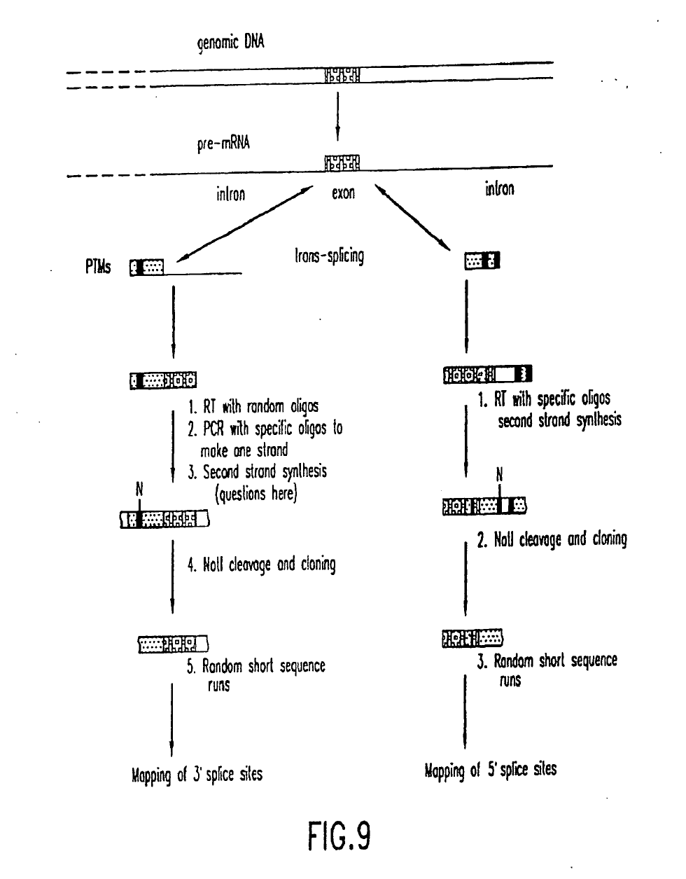

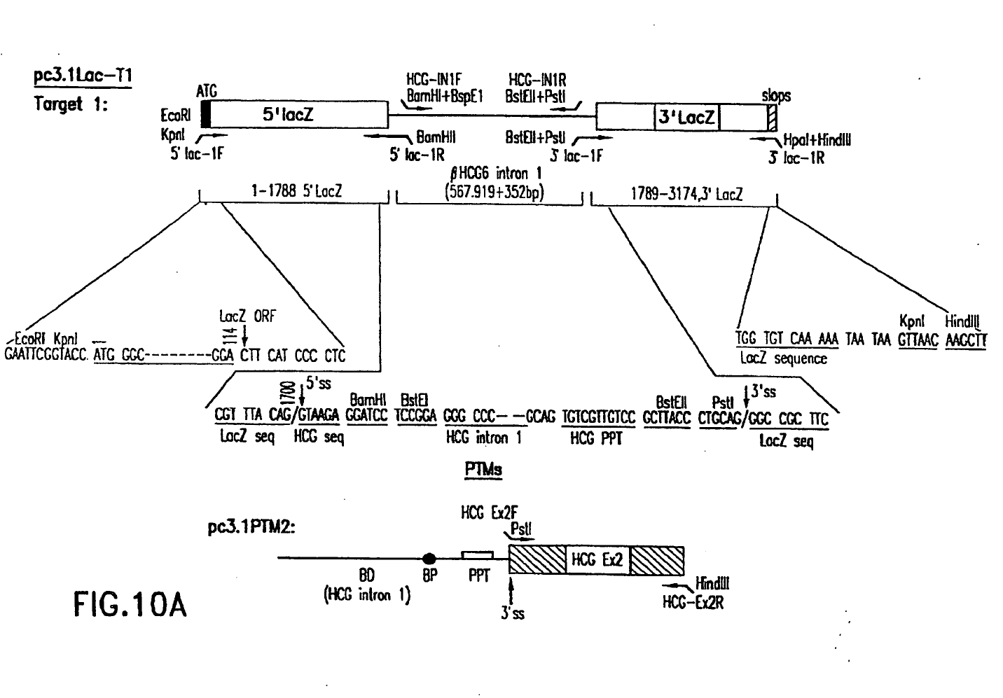

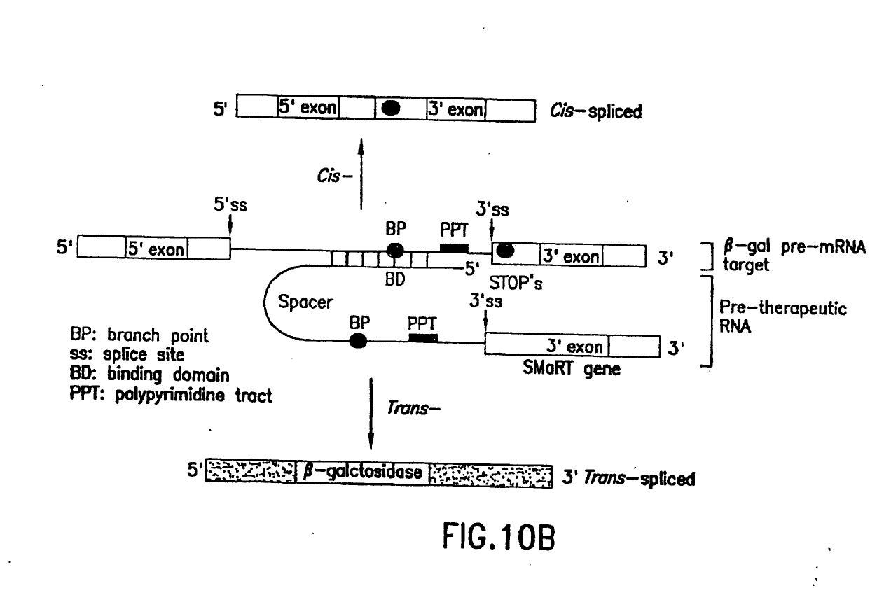

By way of example, FIG. 9 is a schematic representation of two forms of PTMs which can be utilized to map intron-exon boundaries. The PTM on the left is capable of non-specifically trans-splicing into a pre-mRNA 3′ splice site, while the PTM on the right is capable of trans-splicing into a pre-mRNA 5′ splice site. Trans-splicing between the PTM and the target pre-mRNA results in the production of a chimeric mRNA molecule having a specific nucleotide sequence “tag” on either the 3′ or 5′ end of an authentic exon.

Following selective purification, a DNA sequencing reaction is then performed using a primer which begins in the tag nucleotide sequence of the PTM and proceeds into the sequence of the tagged exon. The sequence immediately following the last nucleotide of the tag nucleotide sequence represents an exon boundary. For identification of intron-exon tags, the trans-splicing reactions of the invention can be performed either in vitro or in vivo using methods well known to those of skill in the art.

Use of PTM Molecules for Identification of Proteins Expressed in a Cell

In yet another embodiment of the invention, PTM mediated trans-splicing reactions can be used to identify previously undetected and unknown proteins expressed in a cell. This method is especially useful for identification of proteins that cannot be detected by a two-dimensional electrophoresis, or by other methods, due to inter alia the small size of the protein, low concentration of the protein, or failure to detect the protein due to similar migration patterns with other proteins in two-dimensional electrophoresis.

The present invention relates to a method for identifying proteins expressed in a cell comprising (i) contacting a pre-trans-splicing molecule containing a random target binding domain and a nucleotide sequence encoding a peptide tag with a pre-mRNA molecule under conditions in which a portion of the pre-trans-splicing molecule is trans-spliced to a portion of the target pre-mRNA to form a chimeric mRNA encoding a fusion polypeptide or separating it by gel electrophoresis (ii) affinity purifying the fusion polypeptide; and (iii) determining the amino acid sequence of the fusion protein.

To identify proteins expressed in a cell, the PTMs of the invention are genetically engineered to contain: (i) a target binding domain comprising randomized nucleotide sequences; (ii) a 3′ splice region that includes a branch point, pyrimidine tract and a 3′ splice acceptor site and/or a 5′ splice donor site; (iii) a spacer region that separates the PTM splice site from the target binding domain; and (iv) nucleotide sequences encoding a marker or peptide affinity purification tag. Such peptide tags include, but are not limited to, HIS tags (6 histidine consecutive residues) (Janknecht, et al., 1991 Proc. Natl. Acad. Sci. USA 88:8972-8976), glutathione-S-transferase (GST) (Smith, D. B. and Johnson K. S., 1988, Gene 67:31) (Pharmacia) or FLAG (Kodak/IBI) tags (Nisson, J. et al. J. Mol. Recognit., 1996, 5:585-594).

Trans-splicing reactions using such PTMs results in the generation of chimeric mRNA molecules encoding fusion proteins comprising protein sequences normally expressed in a cell linked to a marker or peptide affinity purification tag. The desired goal of such a method is that every protein synthesized in a cell receives a marker or peptide affinity tag thereby providing a method for identifying each protein expressed in a cell.

In a specific embodiment of the invention, PTM expression libraries encoding PTMs having different target binding domains comprising random nucleotide sequences are generated. The desired goal is to create a PTM expression library that is complex enough to produce a PTM capable of binding to each pre-mRNA expressed in a cell. In a preferred embodiment, the library is cloned into a mammalian expression vector that results in one, or at most, a few vectors being present in any one cell.

To identify the expression of chimeric proteins, host cells are transformed with the PTM library and plated so that individual colonies containing one PTM vector can be grown and purified. Single colonies are selected, isolated, and propagated in the appropriate media and the labeled chimeric protein exon(s) fragments are separated away from other cellular proteins using, for example, an affinity purification tag. For example, affinity chromatography can involve the use of antibodies that specifically bind to a peptide tag such as the FLAG tag. Alternatively, when utilizing HIS tags, the fusion proteins are purified using a Ni²⁺ nitriloacetic acid agarose columns, which allows selective elution of bound peptide eluted with imidazole containing buffers. When using GST tags, the fusion proteins are purified using glutathione-S-transferase agarose beads. The fusion proteins can then be eluted in the presence of free glutathione.

Following purification of the chimeric protein, an analysis is carried out to determine the amino acid sequence of the fusion protein. The amino acid sequence of the fusion protein is determined using techniques well known to those of skill in the art, such as Edman Degradation followed by amino acid analysis using HPLC, mass spectrometry or an amino acid analyzation. Once identified, the peptide sequence is compared to those sequences available in protein databases, such as GenBank. If the partial peptide sequence is already known, no further analysis is done. If the partial protein sequence is unknown, then a more complete sequence of that protein can be carried out to determine the full protein sequence. Since the fusion protein will contain only a portion of the full length protein, a nucleic acid encoding the full length protein can be isolated using conventional methods. For example, based on the partial protein sequence oligonucleotide primers can be generated for use as probes or PCR primers to screen a cDNA library.

### EXAMPLE

### Production of Trans-Splicing Molecules

The following section describes the production of PTMs and the demonstration that such molecules are capable of mediating trans-splicing reactions resulting in the production of chimeric mRNA molecules.

Materials and Methods

Construction of Pre-mRNA Molecules

Plasmids containing the wild type diphtheria toxin subunit A (DT-A, wild-type accession #K01722) and a DT-A mutant (CRM 197, no enzymatic activity) were obtained from Dr. Virginia Johnson, Food and Drug Administration, Bethesda, Md. (Uchida et al., 1973 J. Biol. Chem 248:3838). For in vitro experiments, DT-A was amplified using primers: DT-1F (5′-GGCGCTGCAGGGCGCTGATGATGTTGTTG) (SEQ ID NO:2); and DT-2R (5′-GGCGAAG CTTGGATCCGACACGATTTCCTGCACAGG) (SEQ ID NO:3), cut with PstI and HindIII, and cloned into PstI and HindIII digested pBS(−) vector (Stratagene, La Jolla, Calif.). The resulting clone, pDTA was used to construct the individual PTMs. (1) pPTM+: Targeted construct. Created by inserting IN3-1 (5′AATTCTCTAGATGCTT CACCCGGGCCTGACTCGAGTACTAACTGGTACCTCTTCTTTTTTTTCCTGCA) (SEQ ID NO:4) and IN2-4 (5′-GGAAAAAAAAGAAGAGGTACCAGTTAGTACTCGAGTCAGG CCCGGGTGAAGCATCTAGAG) (SEQ ID NO:5) primers into EcoRI and Pstl digested pDTA. (2) pPTM+Sp: As pPTM+ but with a 30 bp spacer sequence between the BD and BP. Created by digesting pPTM+ with XhoI and ligating in the oligonucleotides, spacer S (5′-TCGAGCAACGTTATAATAATGTTC) (SEQ ID NO:6) and spacer AS (5′-TCGAGAACATTATT ATAACGTTGC) (SEQ ID NO:7). For in vivo studies, an EcoRI and HindIII fragment of pcPTM+Sp was cloned into mammalian expression vector pcDNA3.1 (Invitrogen), under the control of a CMV promoter. Also, the methionine at codon 14 was changed into isoleucine to prevent initiation of translation. The resulting plasmid was designated as pcPTM+Sp. (3) pPTM+CRM: As pPTM+Sp but the wild type DT-A was substituted with CRM mutant DT-A (T. Uchida, et al., 1973, J. Biol. Chem. 248:3838). This was created by PCR amplification of a DT-A mutant (mutation at G52E) using primers DT-1F and DT-2R. For in vivo studies, an EcoRI HindIII fragment of PTM+CRM was cloned into pc3.1DNA that resulted in pcPTM+ARM. (4) PTM−: Non-targeted construct. Created by digestion of PTM+ with EcoRI and Pst I, gel purified to remove the binding domain followed by ligation of the oligonucleotides, IN-5 (5′-ATCTCTAGATCAGGCCCGGGTGAAGCC CGAG) (SEQ ID NO:8) and IN-6 (5′-TGCTTCACCC GGGCCTGATCTAGAG) (SEQ ID NO:9). (5) PTM−Sp, is an identical version of the PTM−, except it has a 30 bp spacer sequence at the PstI site. Similarly, the splice mutants [Py(−)AG(−) and BP(−)Py(−)AG(−)] and safety variants [PTM+SF-Py1, PTM+SF-Py2, PTM+SFBP3 and PTM+SFBP3-Py1] were constructed either by insertion or deletion of specific sequences (see Table 1).

TABLE 1

Binding/non-binding domain, BP, PPT and 3′ as sequences of different
PTMs.

| | | | | |
|---|---|---|---|---|
| PTM construct | BD/NBD | BP | PPT | 3′ss |

| | | | | |
|---|---|---|---|---|
| PTM + Sp (targeted) | TGCTTCACCCGGGCCTGA | TACTAAC | CTCTTCTTTTTTTTCC | CAG |
| | (SEQ ID NO: 10) | | (SEQ ID NO: 11) | |
| PTM − Sp (non-targeted) | CAACGTTATAATAATGTT | TACTAAC | CTCTTCTTTTTTTTCC | CAG |
| | (SEQ ID NO: 12) | | (SEQ ID NO: 11) | |
| PTM + Py(−)AG(−)BP(−) | TGCTTCACCCGGGCCTGA | GGCTGAT | CTGTGATTAATAGCGG | ACG |
| | (SEQ ID NO: 10) | | (SEQ ID NO: 13) | |
| PTM + Py(−)AG(−) | TGCTTCACCCGGGCCTGA | TACTAAC | CCTGGACGCGGAAGT | ACG |
| | (SEQ ID NO: 10) | | T | |
| | | | (SEQ ID NO: 14) | |
| PTM + SF | CTGGGACAAGGACACTGCTT | TACTAAC | CTTCTGTTTTTTTCTC | CAG |
| | CACCCGGTTAGTAGACCACA | | (SEQ ID NO: 16) | |
| | GCCCTGAAGCC | | | |
| | (SEQ ID NO: 15) | | | |
| PTM + SF − Py1 | As in PTM + SF | TACTAAC | CTTCTGTATTATTCTC | CAG |
| | | | (SEQ ID NO: 17) | |
| PTM + SF − Py2 | As in PTM + SF | TACTAAC | GTTCTGTCCTTGTCTC | CAG |
| | | | (SEQ ID NO: 18) | |
| PTM + SF − BP3 | As in PTM + SF | TGCTGAC | CTTCTGTTTTTTTCTC | CAG |
| | | | (SEQ ID NO: 16) | |
| PTM + SFBP3 − Py1 | As in PTM + SF | TGCTGAC | CTTCTGTATTATTCTC | CAG |
| | | | (SEQ ID NO: 17) | |

Nucleotides in bold indicate the mutations compared to normal BP, PPT and 3′ splice site. Branch site A is underlined. The nucleotides in italics indicates the mismatch introduced into safety BD to mask the BP sequence in the PTM.

A double-trans-splicing PTM construct (DS-PTM) was also made adding a 5′ splice site and a second target binding domain complementary to the second intron of βHCG pre-mRNA to the 3′ end of the toxin coding sequence of PTM+SF (Figure A).

βHCG6 Target Pre-mRNA

To produce the in vitro target pre-mRNA, a SacI fragment of βHCG gene 6 (accession #X00266) was cloned into pBS(−). This produced an 805 bp insert from nucleotide 460 to 1265, which includes the 5′ untranslated region, initiation codon, exon 1, intron 1, exon 2, and most of intron 2. For in vivo studies, an EcoRI and BamHI fragment was cloned into mammalian expression vector (pc3.1DNA), producing βHCG6.

mRNA Preparation

For in vitro splicing experiments, βHCG6, β-globin pre-mRNA and different PTM mRNAs were synthesized by in vitro transcription of BamHI and HindIII digested plasmid DNAs respectively, using T7 mRNA polymerase (Pasman & Garcia-Blanco, 1996, Nucleic Acids Res. 24:1638). Synthesized mRNAs were purified by electrophoresis on a denaturing polyacrylamide gel, and the products were excised and eluted.

In Vitro Splicing

PTMs and target pre-mRNA were annealed by heating at 98 EC followed by slow cooling to 30-34 EC. Each reaction contained 4 μl of annealed mRNA complex (100 ng of target and 200 ng of PTM), 1× splice buffer (2 mM MgCl₂, 1 mM ATP, 5 mM creatinine phosphate, and 40 mM KCl) and 4 μl of HeLa splice nuclear extract (Promega) in a 12.5 μl final volume. Reactions were incubated at 30° C. for the indicated times and stopped by the addition of an equal volume of high salt buffer (7 M urea, 5% SDS, 100 mM LiCl, 10 mM EDTA and 10 mM Tris-HCl, pH 7.5). Nucleic acids were purified by extraction with phenol:chloroform:isoamyl alcohol (50:49:1) followed by ethanol precipitation.

Reverse Transcription-PCR Reactions

RT-PCR analysis was performed using EZ-RT PCR kit (Perkin-Elmer, Foster City, Calif.). Each reaction contained 10 ng of cis- or trans-spliced mRNA, or 1-2 μg of total mRNA, 0.1 μl of each 3′ and 5′ specific primer, 0.3 mM of each dNTP, 1×EZ buffer (50 mM bicine, 115 mM potassium acetate, 4% glycerol, pH 8.2), 2.5 mM magnesium acetate and 5 U of rTth DNA polymerase in a 50 μl reaction volume. Reverse transcription was performed at 60° C. for 45 min followed by PCR amplification of the resulting cDNA as follows: one cycle of initial denaturation at 94° C. for 30 sec, and 25 cycles of denaturation at 94° C. for 18 sec and annealing and extension at 60° C. for 40 sec, followed by a 7 min final extension at 70° C. Reaction products were separated by electrophoresis in agarose gels.

Primers used in the study were as follows:

(SEQ ID NO: 19)

| | |
|---|---|
| DT-1F: | GGCGCTGCAGGGCGCTGATGATGTTGTTG |

(SEQ ID NO: 20)

| | |
|---|---|
| DT-2R: | GGCGAAGCTTGGATCCGACACGATTTCCTGCACAGG |

(SEQ ID NO: 21)

| | |
|---|---|
| DT-3R: | CATCGTCATAATTTCCTTGTG |

(SEQ ID NO: 22)

| | |
|---|---|
| DT-4R: | ATGGAATCTACATAACCAGG |

(SEQ ID NO: 23)

| | |
|---|---|
| DT-5R: | GAAGGCTGAGCACTACACGC |

(SEQ ID NO: 24)

| | |
|---|---|
| HCG-R2: | CGGCACCGTGGCCGAAGTGG |

(SEQ ID NO: 25)

| | |
|---|---|
| Bio-HCG-F: | ACCGGAATTCATGAAGCCAGGTACACCAGG |

(SEQ ID NO: 26)

| | |
|---|---|
| β-globulin-F: | GGGCAAGGTGAACGTGGATG |

(SEQ ID NO: 27)

| | |
|---|---|
| β-globulin-R: | ATCAGGAGTGGACAGATCC |

Cell Growth, Transfection and mRNA Isolation

Human lung cancer cell line H1299 (ATCC accession # CRL-5803) was grown in RPMI medium supplemented with 10% fetal bovine serum at 37° C. in a 5% CO₂ environment. Cells were transfected with pcSp+CRM (CRM is a non-functional toxin), a vector expressing a PTM, or vector alone (pcDNA3.1) using lipofectamine reagent (Life Technologies, Gaithersburg, Md.). The assay was scored for neomycin resistance (neo^{r}) colony formation two weeks after transfection. Four neo^{r} colonies were selected and expanded under continued neo selection. Total cellular mRNA was isolated using RNA exol (BioChain Institute, Inc., San Leandro, Calif.) and used for RT-PCR.

Trans-Splicing in Tumors in Nude Mice

Eleven nude mice were bilaterally injected (except B10, B11 and B12 had 1 tumor) into the dorsal flank subcutaneous space with 1×10⁷ H1299 human lung tumor cells (day 1). On day 14, the mice were given an appropriate dose of anesthesia and injected with, or without electroporation (T820, BTX Inc., San Diego, Calif.) in several orientations with a total volume of 100 Φl of saline containing 100 Φg pcSp+CRM with or without pcβHCG6 or pcPTM+Sp. Solutions injected into the right side tumors also contained India ink to mark needle tracks. The animals were sacrificed 48 hours later and the tumor excised and immediately frozen at −80 EC. For analysis, 10 mg of each tumor was homogenized and mRNA was isolated using a Dynabeads mRNA direct kit (Dynal) following the manufacturers directions. Purified mRNA (2 Φl of 10 Φl total volume) was subjected to RT-PCR using βHCG-F and DT-5R primers as described earlier. All samples were re-amplified using DT-3R, a nested DT-A primer and biotinylated βHCG-F and the products were analyzed by electrophoresis on a 2% agarose gel. Samples that produced a band were processed into single stranded DNA using M280 Streptavidin Dynabeads and sequenced using a toxin specific primer (DT-3R).

Results

Synthesis of PTM

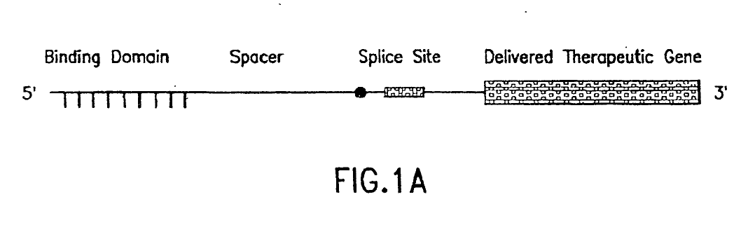

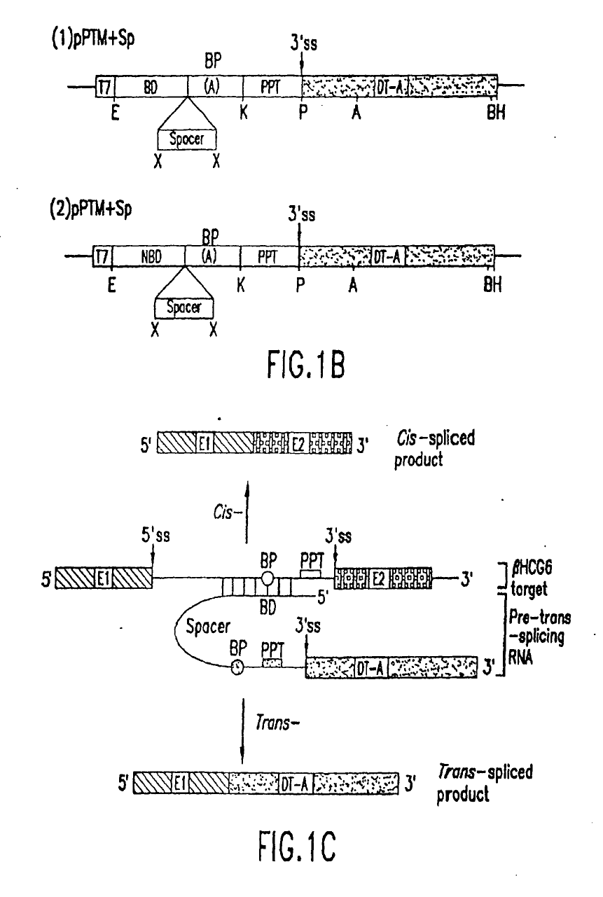

A prototypical trans-splicing mRNA molecule, pcPTM+Sp (FIG. 1A) was constructed that included: an 18 nt target binding domain (complementary to βHCG6 intron 1), a 30 nucleotide spacer region, branch point (BP) sequence, a polypyrimidine tract (PPT) and an AG dinucleotide at the 3′ splice site immediately upstream of an exon encoding diphtheria toxin subunit A (DT-A) (Uchida et al., 1973, J. Biol. Chem. 248:3838). Later DT-A exons were modified to eliminate translation initiation sites at codon 14. The PTM constructs were designed for maximal activity in order to demonstrate trans-splicing; therefore, they included potent 3′ splice elements (yeast BP and a mammalian PPT) (Moore et al., 1993, In The mRNA World, R. F. Gesteland and J. F. Atkins, eds. (Cold Spring Harbor, N.Y.: Cold Spring Harbor Laboratory Press). βHCG6 pre-mRNA (Talmadge et al., 1984, Nucleic Acids Res. 12:8415) was chosen as a model target as this gene is expressed in most tumor cells. It is not expressed in normal adult cells, with the exception of some in the pituitary gland and gonads. (Acevedo et al., 1992, Cancer 76:1467; Hoon et al., 1996, Int J. Cancer 69:369; Bellet et al., 1997, Cancer Res. 57:516). As shown in FIG. 1C, pcPTM+Sp forms conventional Watson-Crick base pairs by its binding domain with the 3′ end of βHCG6 intron 1, masking the intronic 3′ splice signals of the target. This feature is designed to facilitate trans-splicing between the target and the PTM.

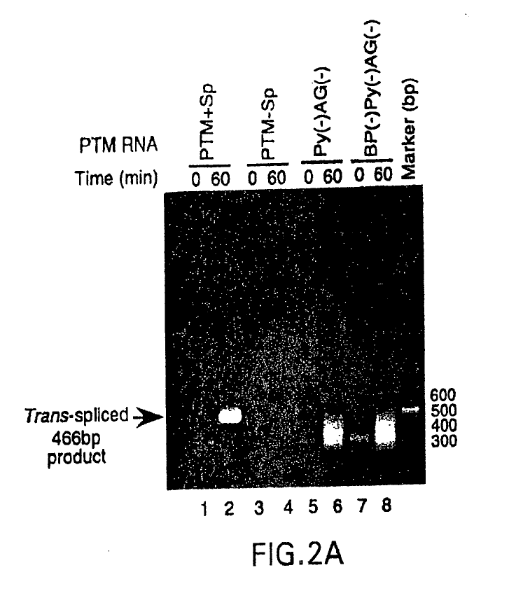

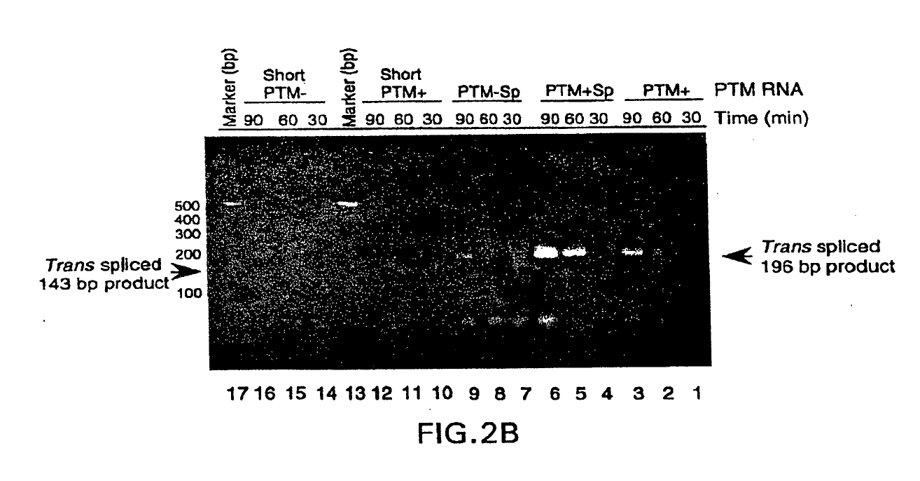

HeLa nuclear extracts were used in conjunction with established splicing procedures (Pasman & Garcia-Blanco, 1996, Nucleic Acids Res. 24:1638) to test if a PTM construct could invade the βHCG6 pre-mRNA target. The products of in vitro trans-splicing were detected by RT-PCR, using primers specific for chimeric mRNA molecules. The predicted product of a successful trans-splicing reaction is a chimeric mRNA comprising the first exon of βHCG6, followed immediately by the exon contributed from pcPTM+Sp encoding DT-A (FIG. 1C). Such chimeric mRNAs were readily detected by RT-PCR using primers βHCG-F (specific to βHCG6 exon 1) and DT-3R (specific to DT-A, FIG. 2A, lanes 1-2). At time zero or in the absence of ATP, no 466 bp product was observed, indicating that this reaction was both ATP and time dependent.

The target binding domain of pcPTM+Sp contained 18 nucleotides complementary to βHCG6 intron 1 pre-mRNA and demonstrated efficient trans-splicing (FIG. 2A, lanes 1-2). Trans-splicing efficiency decreased at least 8 fold (FIG. 2, lanes 3-4) using non-targeted PTM-Sp, which contains a non-complementary 18 nucleotide “non-binding domain”. Trans-splicing efficiencies of PTM mRNAs with or without a spacer between the binding domain and BP were also compared. This experiment demonstrated a significant increase in the efficiency of trans-splicing by the addition of a spacer (FIG. 2B, lanes 2+5). To facilitate the recruitment of splicing factors required for efficient trans-splicing, some space may be needed between the 3′ splice site and the double-stranded secondary structure produced by the binding domain/target interaction.

To investigate the effect of PTM length on trans-splicing specificity, shorter PTMs were synthesized from AccI cut PTM plasmid (see FIG. 1). This eliminated 479 nt from the 3′ end of the DT-A coding sequence. FIG. 2B shows the trans-splicing ability of a targeted short PTM(+) (lanes 10-12), compared to a non-targeted short PTM(−) (lanes 14-17). Short PTM+ produced substantially more trans-spliced product (FIG. 2B, lane 12) than its counterpart, non-targeted short PTM (FIG. 2B, lane 17). These experiments indicate that longer PTMs may have increased potential to mediate trans-splicing non-specifically.

Accuracy of PTM Spliceosome Mediated Trans-Splicing

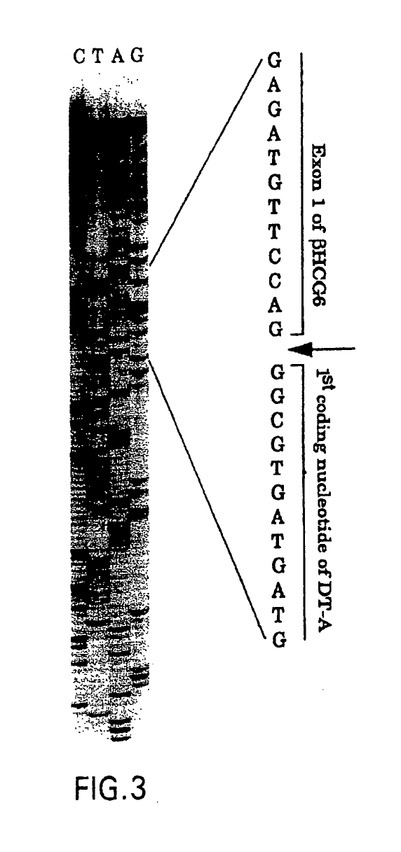

To confirm that trans-splicing between the pcPTM+Sp and βHCG6 target is precise, RT-PCR amplified product was produced using 5′ biotinylated βHCG-F and non-biotinylated DT-3R primers. This product was converted into single stranded DNA and sequenced directly with primer DT-3R (DT-A specific reverse primer) using the method of Mitchell and Merril (1989, Anal. Biochem. 178:239). Trans-splicing occurred exactly between the predicted splice sites (FIG. 3), confirming that a conventional pre-mRNA can be invaded by an engineered PTM construct during splicing; moreover, this reaction is precise.

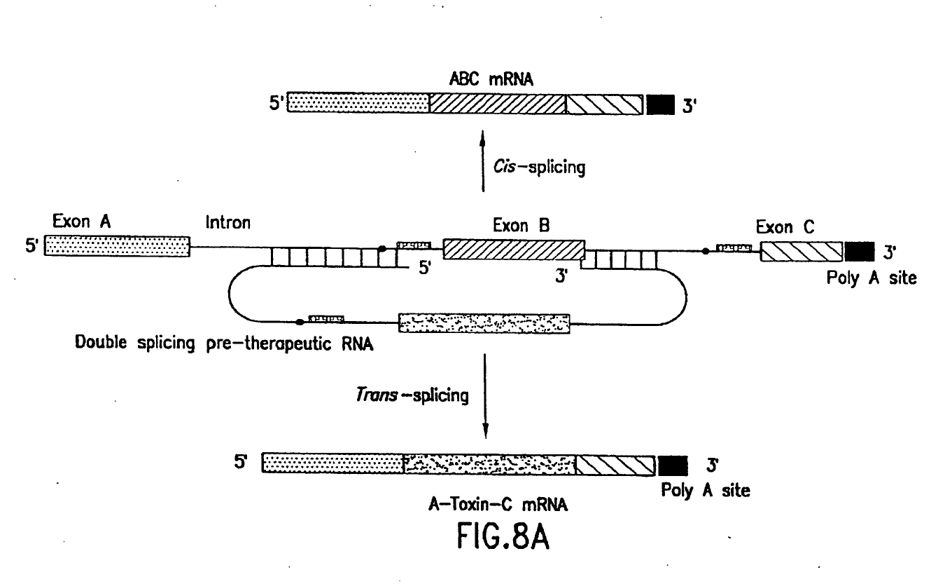

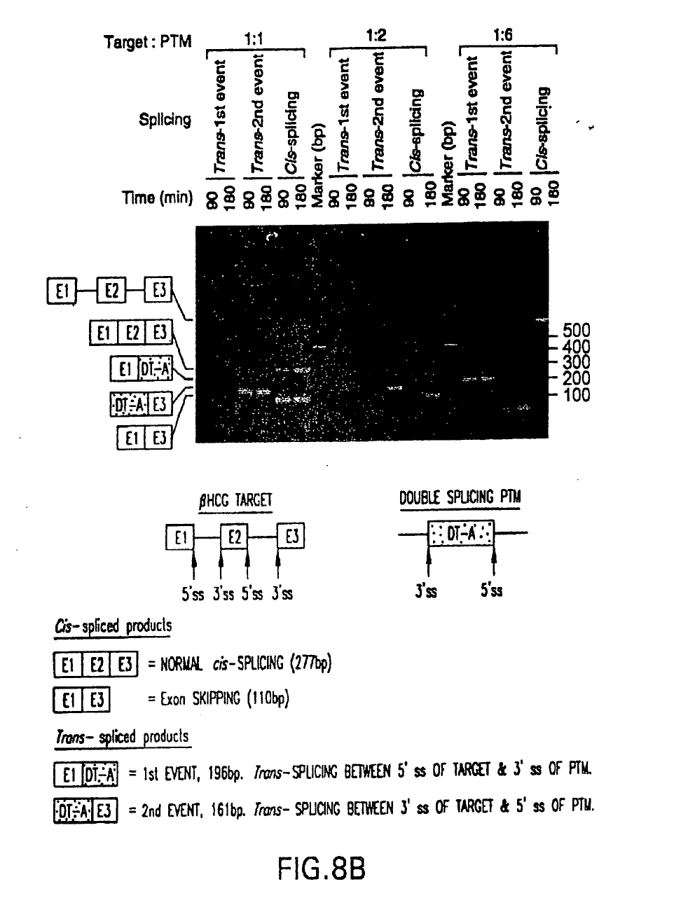

In addition selective trans-splicing of a double splicing PTM (DS-PTM) was observed (FIG. 8B). The DS-PTM can produce trans-splicing by contributing either a 3′ or 5′ splice site. Further, DS-PTMs can be constructed which will be capable of simultaneously double-trans-splicing, at both a 3′ and 5′ site, thereby permitting exon replacement. FIG. 8B demonstrates that in this construct the 5′ splice site is most active at a 1:1 concentration of target βHCG pre-mRNA:DS-PTM. At a 1:6 ratio the 3′ splice site is more active.

3′ Splice Sites are Essential for PTM Trans-Splicing

In general, the 3′ splice site contains three elements: 1) a BP sequence located 5′ of the acceptor site, 2) a PPT consisting of a short run of pyrimidine residues, and 3) a YAG trinucleotide splice site acceptor at the intron-exon border (Senaphthy et al., 1990, Cell 91:875; Moore et al., 1993). Deletion or alteration of one of these sequence elements are known to either decrease or abolish splicing (Aebi et al., 1986; Reed & Maniatis 1988, Genes Dev. 2:1268; Reed, 1989, Genes Dev. 3:2113; Roscigno et al., 1993, J. Biol. Chem. 268:11222; Coolidge et al., 1997, Nucleic Acids Res. 25:888). The role of these conserved elements in targeted trans-splicing was addressed experimentally. In one case [(BP(−)Py(−)AG(−)], all three cis elements (BP, PPT and AG dinucleotide) were replaced by random sequences. A second splicing mutant [(Py(−)AG(−)] was constructed in which the PPT and the 3′ splice site acceptor were mutated and substituted by random sequences. Neither construct was able to support trans-splicing in vitro (FIG. 2A, lanes 5-8), suggesting that, as in the case of conventional cis-splicing, the PTM trans-splicing process also requires a functional BP, PPT and AG acceptor at the 3′ splice site.

Development of a “Safety” Splice Site to Increase Specificity

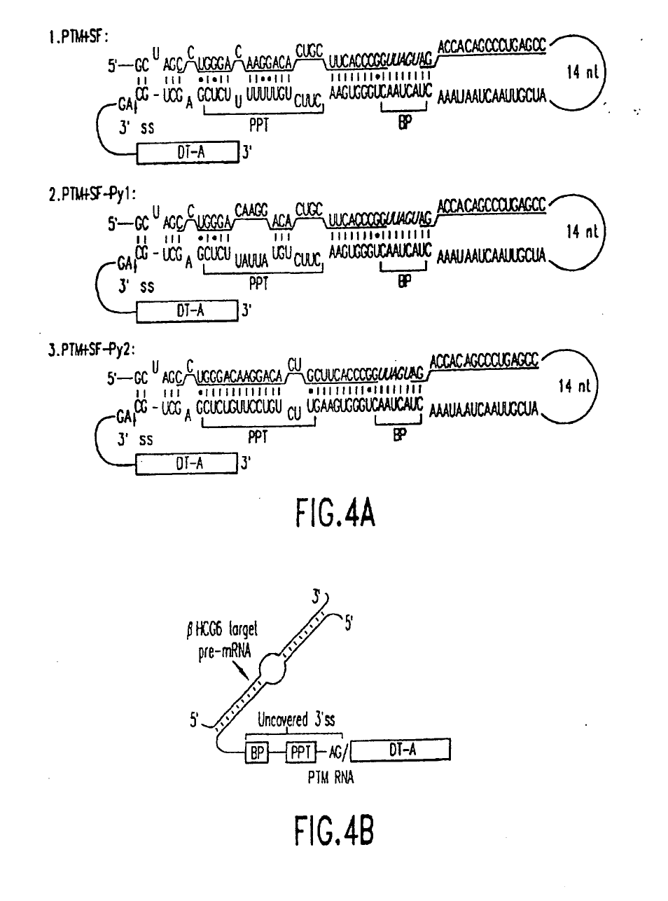

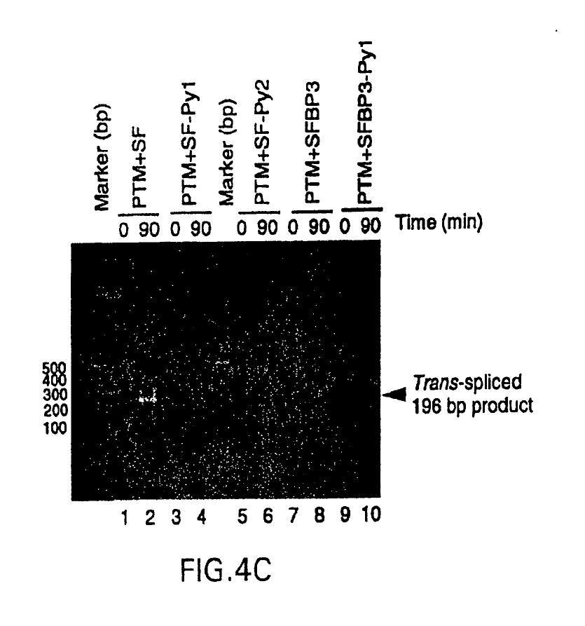

To improve the levels of target specificity achieved by the inclusion of a binding domain or by shortening the PTM, the target-binding domain of several PTM constructs was modified to create an intra-molecular stem to mask the 3′ splice site (termed a “safety PTM”). The safety stem is formed by portions of the binding domain that partially base pair with regions of the PTM 3′ splice site or sequences adjacent to them, thereby blocking the access of spliceosomal components to the PTM 3′ splice site prior to target acquisition (FIG. 4A, PTM+SF). Base pairing between free portions of the PTM binding domain and βHCG6 target region unwinds the safety stem, allowing splicing factors such as U2AF to bind to the PTM 3′ splice site and initiate trans-splicing (FIG. 4B).

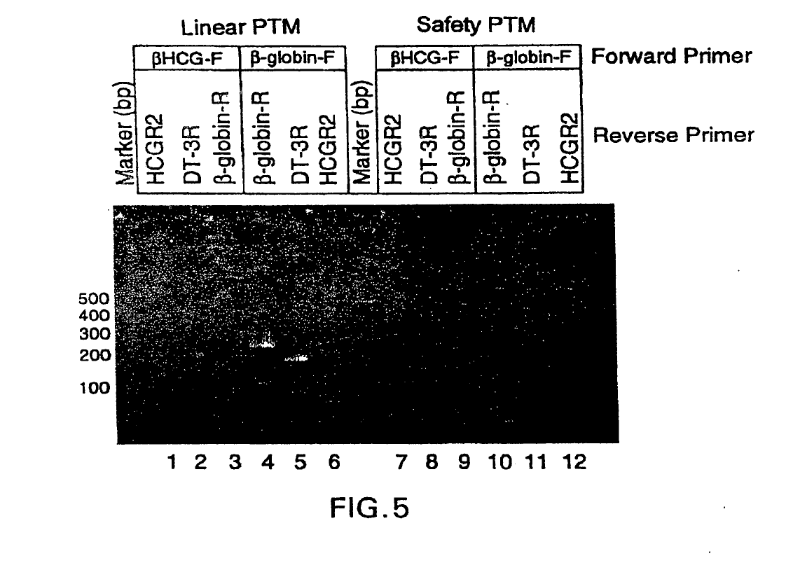

This concept was tested in splicing reactions containing either PTM+SF (safety) or pcPTM+Sp (linear), and both target (βHCG6) and non-target (β-globin) pre-mRNA. The spliced products were subsequently analyzed by RT-PCR and gel electrophoresis. Using βHCG-F and DT-3R primers, the specific 196 bp trans-spliced band was demonstrated in reactions containing βHCG target and either linear PTM (pcPTM+Sp, FIG. 5, lane 2) or safety PTM (PTM+SF, FIG. 5, lane 8). Comparison of the targeted trans-splicing between linear PTM (FIG. 5, lane 2) and safety PTM (FIG. 5, lane 8) demonstrated that the safety PTM trans-spliced less efficiently than the linear PTM.

Non-targeted reactions were amplified using β-globin-F (specific to exon 1 of β-globin) and DT-3R primers. The predicted product generated by non-specific PTM trans-splicing with β-globin pre-mRNA is 189 bp. Non-specific trans-splicing was evident between linear PTM and β-globin pre-mRNA (FIG. 5, lane 5). In contrast, non-specific trans-splicing was virtually eliminated by the use of safety PTM (FIG. 5, lane 11). This was not unexpected, since the linear PTM was designed for maximal activity to prove the concept of spliceosome-mediated trans-splicing. The open structure of the linear PTM combined with its potent 3′ splice sites strongly promotes the binding of splicing factors. Once bound, these splicing factors can potentially initiate trans-splicing with any 5′ splice site, in a process similar to trans-splicing in trypanosomes. The safety stem was designed to prevent splicing factors, such as U2AF from binding to the PTM prior to target acquisition. This result is consistent with a model that base-pairing between the free portion of the binding domain and the βHCG6 target unwinds the safety stem (by mRNA-mRNA interaction), uncovering the 3′ splice site, permitting the recruitment of splicing factors and initiation of trans-splicing. No trans-splicing was detected between β-globin and βHCG6 pre-mRNAs (FIG. 5, lanes 3, 6, 9 and 12).

In Vitro Trans-Splicing of Safety PTM and Variants

To better understand the role of cis-elements at the 3′ splice site in trans-splicing a series of safety PTM variants were constructed in which either the PPT was weakened by substitution with purines and/or the BP was modified by base substitution (see Table I). In vitro trans-splicing efficiency of the safety (PTM+SF) was compared to three safety variants, which demonstrated a decreased ability to trans-splice. The greatest effect was observed with variant 2 (PTM+SFPy2), which was trans-splicing incompetent (FIG. 4C, lanes 5-6). This inhibition of trans-splicing may be attributed to a weakened PPT and/or the higher Tm of the safety stem. In contrast, variations in the BP sequence (PTM+SFBP3) did not markedly effect trans-splicing (FIG. 4C, lanes 7-8). This was not surprising since the modifications introduced were within the mammalian branch point consensus range YNYURAC (where Y=pyrimidine, R=purine and N=any nucleotide) (Moore et al., 1993). This finding indicates that the branch point sequence can be removed without affecting splicing efficiency. Alterations in the PPT (PTM+SF-Py1) decreased the level of trans-splicing (lanes 3-4). Similarly, when both BP and PPT were altered PTM+SFBP3-Py1, they caused a further reduction in trans-splicing (FIG. 4C, lanes 9-10). The order of trans-splicing efficiency of these safety variants is PTM+SF>PTM+SFBP3>PTM+SFPy1>PTM+SFBP3-Py1>PTM+SFPy2. These results confirm that both the PPT and BP are important for efficient in vitro trans-splicing (Roscigno et al., 1993, J. Biol. Chem. 268:11222).

Competition Between Cis- and Trans-Splicing

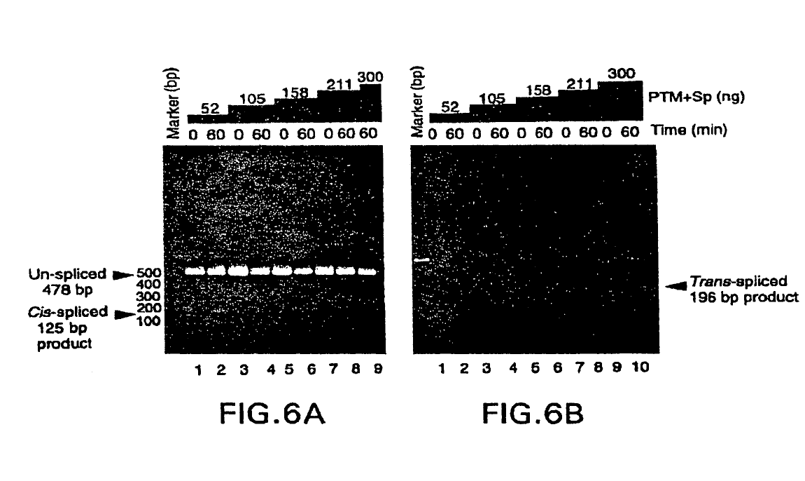

To determine if it was possible to block pre-mRNA cis-splicing by increasing concentrations of PTM, experiments were performed to drive the reaction towards trans-splicing. Splicing reactions were conducted with a constant amount of βHCG6 pre-mRNA target and various concentrations of trans-splicing PTM. Cis-splicing was monitored by RT-PCR using primers to βHCG-F (exon 1) and βHCG-R2 (exon 2). This amplified the expected 125 bp cis-spliced and 478 bp unspliced products (FIG. 6A). The primers βHCG-F and DT-3R were used to detect trans-spliced products (FIG. 6B). At lower concentrations of PTM, cis-splicing (FIG. 6A, lanes 1-4) predominated over trans-splicing (FIG. 6B, lanes 1-4). Cis-splicing was reduced approximately by 50% at a PTM concentration 1.5 fold greater than target. Increasing the PTM mRNA concentration to 3 fold that of target inhibited cis-splicing by more than 90% (FIG. 6A, lanes 7-9), with a concomitant increase in the trans-spliced product (FIG. 6B, lanes 6-10). A competitive RT-PCR was performed to simultaneously amplify both cis and trans-spliced products by including all three primers (βHCG-F, HCG-R2 and DT-3R) in a single reaction. This experiment had similar results to those seen in FIG. 6, demonstrating that under in vitro conditions, a PTM can effectively block target pre-mRNA cis-splicing and replace it with the production of an engineered trans-spliced chimeric mRNA.

Trans-Splicing in Tissue Culture

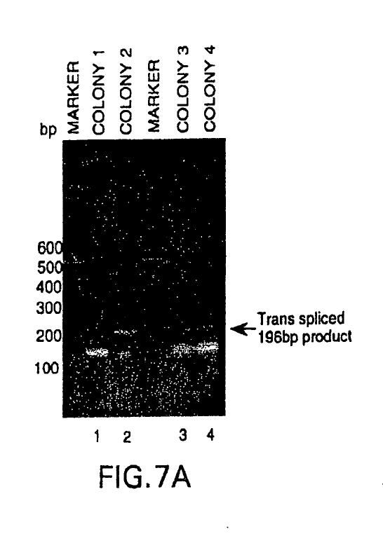

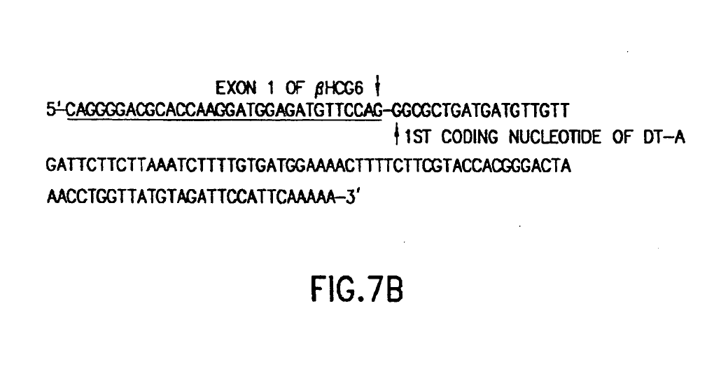

To demonstrate the mechanism of trans-splicing in a cell culture model, the human lung cancer line H1299 (βHCG6 positive) was transfected with a vector expressing SP+CRM (a non-functional diphtheria toxin) or vector alone (pcDNA3.1) and grown in the presence of neomycin. Four neomycin resistant colonies were individually collected after 14 days and expanded in the continued presence of neomycin. Total mRNA was isolated from each clone and analyzed by RT-PCR using primers βHCG-F and DT-3R. This yielded the predicted 196 bp trans-spliced product in three out of the four selected clones (FIG. 7A, lanes 2, 3 and 4). The amplified product from clone #2 was directly sequenced, confirming that PTM driven trans-splicing occurred in human cells exactly at the predicted splice sites of endogenously expressed βHCG6 target exon 1 and the first nucleotide of DT-A (FIG. 7B).

Trans-Splicing in an In Vivo Model

To demonstrate the mechanism of trans-splicing in vivo, the following experiment was conducted in athymic (nude) mice. Tumors were established by injecting 10⁷ H1299 cells into the dorsal flank subcutaneous space. On day 14, PTM expression plasmids were injected into tumors. Most tumors were then subjected to electroporation to facilitate plasmid delivery (see Table 2, below). After 48 hrs, tumors were removed, poly-A mRNA was isolated and amplified by RT-PCR. Trans-splicing was detected in 8 out of 19 PTM treated tumors. Two samples produced the predicted trans-spliced product (466 bp) from mRNA after one round of RT-PCR. Six additional tumors were subsequently positive for trans-splicing by a second PCR amplification using a nested set of primers that produced the predicted 196 bp product (Table 2). Each positive sample was sequenced, demonstrating that βHCG6 exon 1 was precisely trans-spliced to the coding sequence of DT-A (wild type or CRM mutant) at the predicted splice sites. Six of the positive samples were from treatment groups that received cotransfected plasmids, pcPTM+CRM and pcHCG6, which increased the concentration of target pre-mRNA. This was done to enhance the probability of detecting trans-spliced events. The other two positive tumors were from a group that received only pcPTM+Sp (wild type DT-A). These tumors were not transfected with βHCG6 expression plasmid, demonstrating once again, as in the tissue culture model described in Section 6.2.7, that trans-splicing occurred between the PTM and endogenous βHCG6 pre-mRNA produced by tumor cells.

TABLE 2

Trans-splicing in tumors in nude mice.

RT-PCR

| | | | | | | | | | |
|---|---|---|---|---|---|---|---|---|---|
| Mouse | Plasmid | Left | Right | Electroporation | Left | Right | Nested | PCR | Nucleotide Sequence |

| | | | | | | | | | |
|---|---|---|---|---|---|---|---|---|---|
| ⁸B1 | pCMV − Sport | B1-1 | B1-2 | — | − | − | − | − | — |
| B2 | pCMV − Sport | B1-3 | B1-4 | ^{a}1000 V/cm | − | − | − | − | — |
| B3 | pcSp + CRM | B3-1 | B3-2 | ^{a}1000 V/cm | − | − | − | − | — |
| | | B3-3 | B3-4 | ^{a}1000 V/cm | − | − | − | − | — |
| B4 | pcSp + CRM | B4-1 | B4-2 | ^{b}50 V/cm | − | − | − | − | — |
| | | B4-3 | B4-4 | ^{c}25 V/cm | − | − | − | − | — |
| B5 | pcSp + GRM/ | B5-1 | B5-2 | ^{a}1000 V/cm | + | − | + | + | ATGTTCCAG9GGCGTGATGAT |
| | pcHCG6 | | | | | | | | (SEQ ID NO: 65) |
| | | B5-3 | B5-4 | ^{a}1000 V/cm | + | − | + | + | ATGTTCCAG9GGCGTGATGAT |
| | | | | | | | | | (SEQ ID NO: 65) |
| B6 | pcSp + CRM/ | B6-1 | B6-2 | ^{b}50 V/cm | − | − | − | − | — |
| | pcHCG6 | | | | | | | | |
| | | B6-3 | B6-4 | ^{c}25 V/cm | − | − | + | + | ATGTTCCAG9GGCGTGATGAT |
| | | | | | | | | | (SEQ ID NO: 65) |

| | | | | | | |
|---|---|---|---|---|---|---|
| B7 | pc PTM + Sp | B7-1 | ^{a}1000 V/cm | — | — | — |
| B8 | pc PTM + Sp | B8-1 | ^{b}50 V/cm | — | % | ATGTTCCAG9GGCGTGATGAT |
| | | | | | | (SEQ ID NO: 65) |
| ⁹B9 | pc PTM + Sp | B9-1 | — | — | % | ATGTTCCAG9GGCGTGATGAT |
| | | | | | | (SEQ ID NO: 65) |

^{a}6 pulses of 99Φs sets of 3 pulses administered orthogonally
^{b}8 pulses of 10 ms sets of 4 pulses administered orthogonally
^{c}8 pulses of 50 ms sets of 4 pulses administered orthogonally
+: positive for RT-PCR trans-spliced produce
¹: did not receive electroporation

### EXAMPLE

### lacZ Trans-Splicing Model

In order to demonstrate and evaluate the generality of the mechanism of spliceosome mediated targeted trans-splicing between a specific pre-mRNA target and a PTM, a simple model system based on expression of enzyme β-galactosidase was developed. The following section describes results demonstrating successful splicesome mediated targeted trans-splicing between a specific target and a PTM.

Materials and Methods

Primer Sequences

The following primers were used for testing the lacZ model system:

5′ Lac-1F

(SEQ ID NO: 28)

GCATGAATTCGGTACCATGGGGGGGTTCTCATCATCATC
5′ Lac-1R

(SEQ ID NO: 29)

CTGAGGATCCTCTTACCTGTAAACGCCCATACTGAC
3′ Lac-1F

(SEQ ID NO: 30)

GCATGGTAACCCTGCAGGGCGGCTTCGTCTGGGACTGG
3′ Lac-1R

(SEQ ID NO: 31)

CTGAAAGCTTGTTAACTTATTATTTTTGACACCAGACC
3′ Lac-Stop

(SEQ ID NO: 32)

GCATGGTAACCCTGCAGGGCGGCTTCGTCTAATAATGGGACTGGGTG
HCG-In1F

(SEQ ID NO: 33)

GCATGGATCCTCCGGAGGGCCCCTGGGCACCTTCCAC
HCG-In1R

(SEQ ID NO: 34)

CTGACTGCAGGGTAACCGGACAAGGACACTGCTTCACC
HCG-Ex2F

(SEQ ID NO: 35)

GCATGGTAACCCTGCAGGGGCTGCTGCTGTTGCTG
HCG-Ex2R

(SEQ ID NO: 36)

CTGAAAGCTTGTTAACCAGCTCACCATGGTGGGGCAG
Lac-TR1 (Biotin):

(SEQ ID NO: 37)

7-GGCTTTCGCTACCTGGAGAGAC
Lac-TR2

(SEQ ID NO: 38)

GCTGGATGCGGCGTGCGGTCG
HCG-R2:

(SEQ ID NO: 39)

CGGCACCGTGGCCGAAGTGG

Construction of the lacZ Pre-mRNA Target Molecule

The lacZ target 1 pre-mRNA (pc3.1 lacT1) was constructed by cloning of the following three PCR products: (i) the 5′ fragment of lacZ; followed by (ii) βHCG6 intron 1; (iii) and the 3′ fragment of lacZ. The 5′ and 3′ fragment of the lacZ gene were PCR amplified from template pcDNA3.1/His/lacZ (Invitrogen, San Diego, Calif.) using the following primers: 5′ Lac-1F and 5′Lac-1R (for 5′ fragment), and 3′Lac-1F and 3′ Lac-1R (for 3′ fragment). The amplified lacZ 5′ fragment is 1788 bp long which includes the initiation codon, and the amplified 3′ fragment is 1385 bp long and has the natural 5′ and 3′ splice sites in addition to a branch point, polypyrimidine tract and βHCG6 intron 1. The βHCG6 intron 1 was PCR amplified using the following primers: HCG-In1F and HCG-In1R.

The lacZ target 2 is an identical version of lacZ target 1 except it contains two stop codons (TAA TAA) in frame four codons after the 3′splice site. This was created by PCR amplification of the 3′ fragment (lacZ) using the following primers: 3′ Lac-Stop and 3′ Lac 1R and replacing the functional 3′ fragment in lacZ target 1.

Construction of pc3.1 PTM1 and pc3.1 PTM2

The pre-trans-splicing molecule, pc3.1 PTM1 was created by digesting pPTM+Sp with PstI and HindIII and replacing the DNA fragment encoding the DT-A toxin with the a DNA fragment encoding the functional 3′ end of lacZ. This fragment was generated by PCR amplification using the following primers: 3′ Lac-1F and 3′ Lac-1R. For cell culture experiments, an EcoRI and HindIII fragment of pc3.1 PTM2 which contains the binding domain to HCG intron 1, a 30 bp spacer, a yeast branch point (TACTAAC), and strong polypyrimidine tract followed by the lacZ cloned was cloned into pcDNA3.1.

The pre-trans-splicing molecule, pc3.1 PTM2 was created by digesting pPTM+Sp with PstI and HindIII and replacing the DNA fragment encoding the DT-A toxin with the βHCG6 exon 2. βHCG6 exon 2 was generated by PCR amplification using the following primers: HCG-Ex2F and HCG-Ex2R. For cell culture experiments, an EcoRI and HindIII fragment of pc3.1 PTM2 which contains the binding domain to HCG intron 1, a 30 bp spacer, a yeast branch point (TACTAAC), and strong polypyrimidine tract followed by the βHCG6 exon 2 cloned was used.

Co-Transfection of the lacZ Splice Target Pre-mRNA and PTMS into 293T Cells

Human embryonic kidney cells (293T) were grown in DMEM medium supplemented with 10% FBS at 37° C. in a 5% CO₂. Cells were co-transfected with pc3.1 LacT1 and pc3.1 PTM2, or pc3.1 LacT2 and pc3.1 PTM1, using Lipofectamine Plus (Life Technologies, Gaithersburg, Md.) according to the manufacturer's instructions. 24 hours post-transfection, the cells were harvested; total RNA was isolated and RT-PCR was performed using specific primers for the target and PTM molecules. β-galactosidase activity was also monitored by staining the cells using a β-gal staining kit (Invitrogen, San Diego. CA).

Results

The lacZ Splice Target Cis-Splices Efficiently to Produce Functional β-Galactosidase

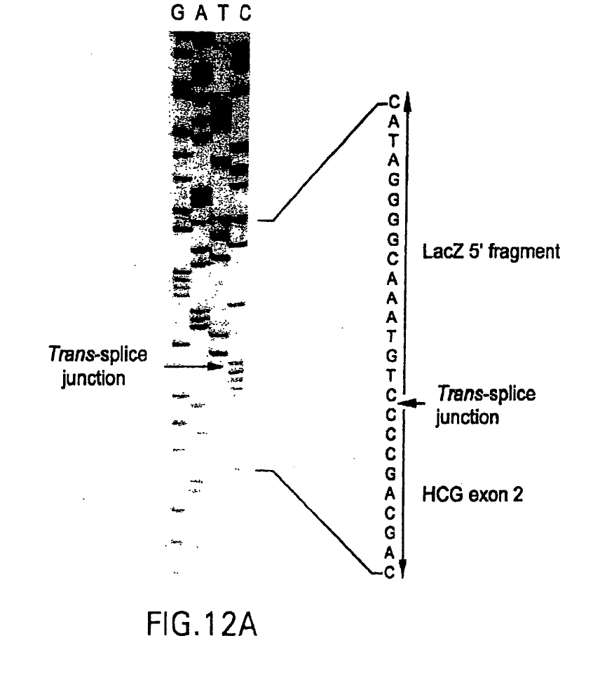

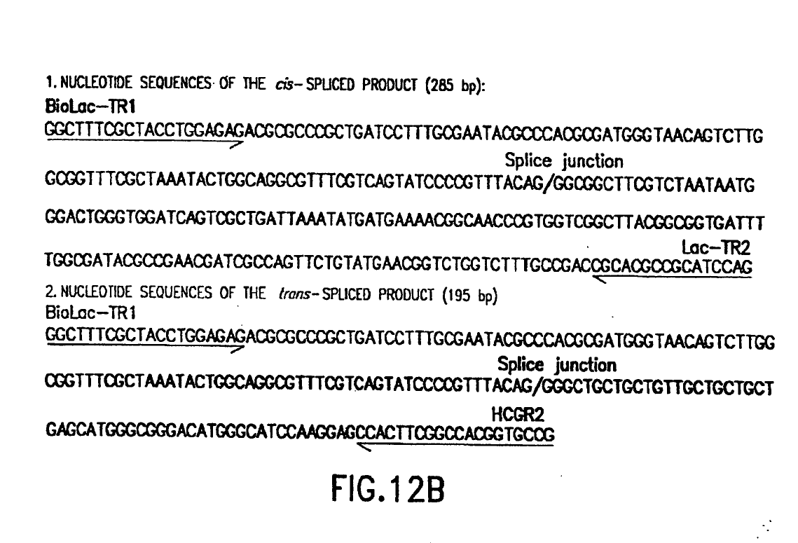

To test the ability of the splice target pre-mRNA to cis-splice efficiently, pc3.1 lacT1 was transfected into 293 T cells using Lipfectamine Plus reagent (Life Technologies, Gaithersburg, Md.) followed by RT-PCR analysis of total RNA. Sequence analysis of the cis-spliced RT-PCR product indicated that splicing was accurate and occurred exactly at the predicted splice sites (FIG. 12B). In addition, accurate cis-splicing of the target pre-mRNA molecule results in formation of a mRNA capable of encoding active β-galactosidase which catalyzes the hydrolysis of β-galactosidase, i.e., X-gal, producing a blue color that can be visualized under a microscope. Accurate cis-splicing of the target pre-mRNA was further confirmed by successfully detecting β-galactosidase enzyme activity.

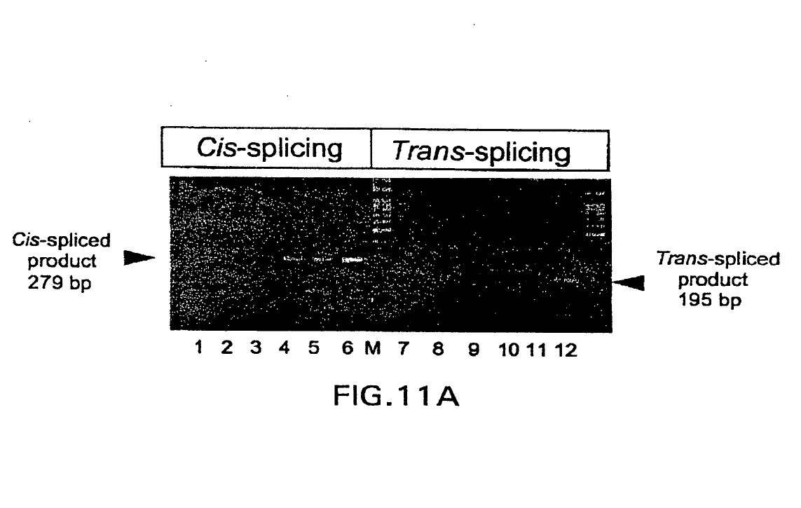

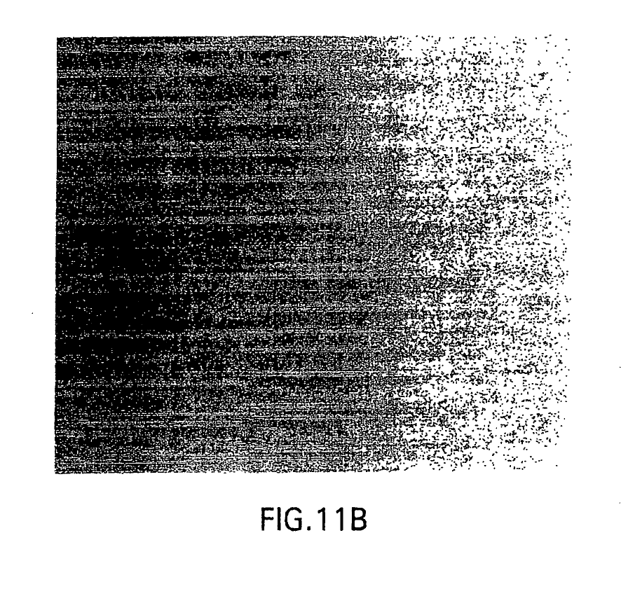

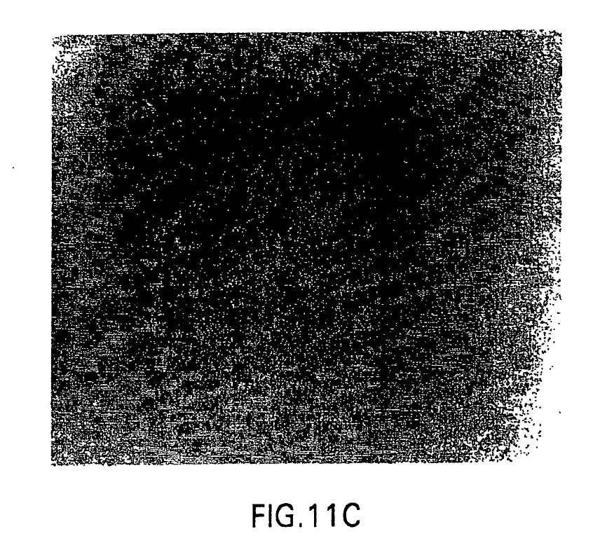

Repair of defective lacZ target 2 pre-mRNA by trans-splicing of the functional 3′ lacZ fragment (PTM1) was measured by staining for β-galactosidase enzyme activity. For this purpose, 293T cells were co-transfected with lacZ target 2 pre-mRNA (containing a defective 3′ fragment) and PTM1 (contain normal 3′ lacZ sequence). 48 hours post-transfection cells were assayed for β-galactosidase enzyme activity. Efficient trans-splicing of PTM1 into the lacZ target 2 pre-mRNA will result in the production of functional β-galactosidase activity. As demonstrated in FIG. 11B-E, trans-splicing of PTM 1 into lacZ target 2 results in restoration of β-galactosidase enzyme activity up to 5% to 10% compared to control.

Targeted Trans-Splicing Between the lacZ Target Pre-mRNA and PTM2

To assay for trans-splicing, lacZ target pre-mRNA and PTM2 were transfected into 293 T cells. Following transfection, total RNA was analyzed using RT-PCR. The following primers were used in the PCR reactions: lacZ-TR1 (lacZ 5′ exon specific) and HCGR2 (βHCGR exon 2 specific). The RT PCR reaction produced the expected 195 bp trans-spliced product (FIG. 11, lanes 2 and 3) demonstrating efficient trans-splicing between the lacZ target pre-mRNA and PTM 2. Lane 1 represents the control, which does not contain PTM 2.

The efficiency of the trans-splicing was also measured by staining for β-galactosidase enzyme activity. To assay for trans-splicing, 293T cells were co-transfected with lacZ target pre-mRNA and PTM 2. 24 hours post-transfection, cells were assayed for β-galactosidase activity. If there is efficient trans-splicing between the target pre-mRNA and the PTM, a chimeric mRNA is produced consisting of the 5′ fragment of the lacZ target pre-mRNA and βHCG6 exon 2 is formed which is incapable of coding for an active β-galactosidase. Results from the co-transfection experiments demonstrated that trans-splicing of PTM2 into lacZ target 1 resulted in the reduction of β-galactosidase activity by compared to the control.

To further confirm that trans-splicing between the lacZ target pre-mRNA and PTM2 is accurate, RT-PCR was performed using 5′ biotinylated lacZ-TR1 and non-biotinylated HCGR2 primers. Single stranded DNA was isolated and sequenced directly using HCGR2 primer (HCG exon 2 specific primer). As evidenced by the sequence of the splice junction, trans-splicing occurred exactly as predicted between the splice sites (FIGS. 12A and 12B), confirming that a conventional pre-mRNA can be invaded by an engineered PTM during splicing, and moreover, that this reaction is precise.

### EXAMPLE

### Correction of the Cystic Fibrosis Transmembrane Regulator Gene

Cystic fibrosis (CF) is one of the most common genetic diseases in the world. The gene associated with CF has been isolated and its protein product deduced (Kerem, B. S. et al., 1989, Science 245:1073-1080; Riordan et al., 1989, Science 245:1066-1073; Rommans, et al., 1989, Science 245:1059-1065). The protein product of the CF associated gene is referred to as the cystic fibrosis trans-membrane conductance regulator (CFTR). The most common disease-causing mutation which accounts for ˜70% of all mutant alleles is a deletion of three nucleotides in exon 10 that encode for a phenylalanine at position 508 (ΔF508). The following section describes the successful repair of the cystic fibrosis gene using spliceosome mediated trans-splicing and demonstrates the feasibility of repairing CFTR in a model system.

Materials and Methods

Pre-Trans-Splicing Molecule

The CFTR pre-trans-splicing molecule (PTM) consists of a 23 nucleotide binding domain complimentary to CFTR intron 9 (3′ end, −13 to −31), a 30 nucleotide spacer region (to allow efficient binding of spliceosomal components), branch point (BP) sequence, polypyrimidine tract (PPT) and an AG dinucleotide at the 3′ splice site immediately upstream of the sequence encoding CFTR exon 10 (wild type sequence containing F508). This initial PTM was designed for maximal activity in order to demonstrate trans-splicing; therefore the PTM included a UACUAAC yeast consensus BP sequence and an extensive PPT. An 18 nucleotide HIS tag (6 histamine codons) was included after wild type exon 10 coding sequence to allow specific amplification and isolation of the trans-spliced products and not the endogenous CFTR. The oligonucleotides used to generate the two fragments included unique restriction sites. (Apal and PstI, and PstI and NotI, respectively) to facilitate directed cloning of amplified DNA into the mammalian expression vector pcDNA3.1.

The Target CFTR Pre-mRNA Mini-Gene

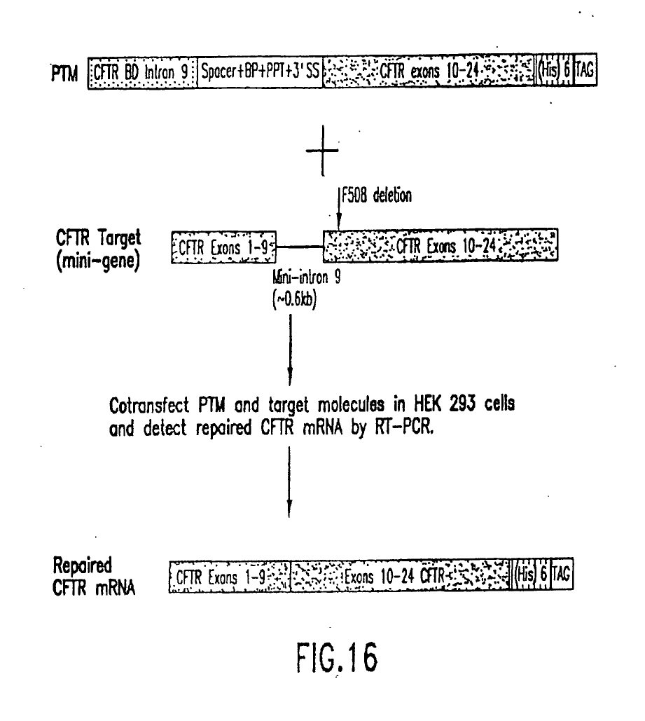

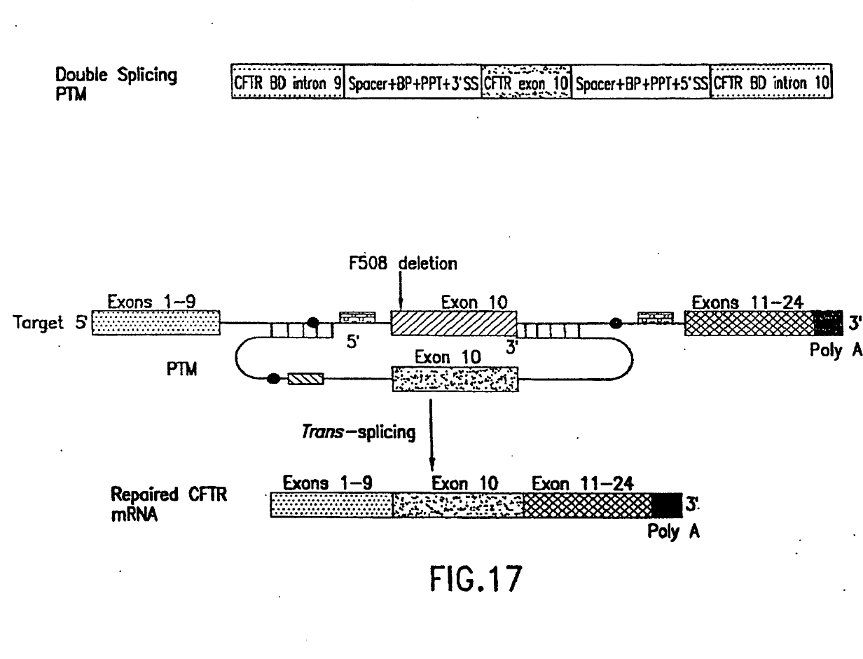

The CFTR mini-gene target is shown in FIG. 13 and consists of CFTR exon 9, the functional 5′ and 3′ regions of intron 9 (260 and 265 nucleotides from each end, respectively); exon 10 [ΔF508]; and the 5′ region of intron 10 (96 nucleotides). In addition, as depicted in FIG. 16, a mini-target gene comprising CFTR exons 1-9 and 10-24 can be used to test the use of spliceosome mediated trans-splicing for correction of the cystic fibrosis mutation. FIG. 17, shows a double splicing PTM that may also be used for correction of the cystic fibrosis mutation. As shown, the double splicing PTM contains CFTR BD intron 9, a spacer, a branch point, a polypyrimidine tract, a 3′ splice site, CFTR exon 10, a spacer, a branch point, a polypyrimidine tract, a 5′ splice site and CFTR BD exon 10.

Oligonucleotides

The following oligonucleotides were used to create CFTR PTM:

Forward CF3

(SEQ ID NO: 40)

```
                ACCT GGGCCC ACC CAT TAT TAG GTC ATT AT
                          ApaI site. Intron 9 CFTR, −12 to −34.
                CCGCGG AAC ATT ATA
Reverse CF4
```

(SEQ ID NO: 41)

```
                ACCT CTGCAGGTGACC CTG CAG GAA AAA AAA GAA G
                          PstI BstEI PPT
Forward CF5
```

(SEQ ID NO: 42)

```
                ACCT CTGCAG ACT TCA CTT CTA ATG ATG AT
                          PstI. Exon 10 CFTR, +1 to +24
Reverse CF6
```

(SEQ ID NO: 43)

```
                ACCT GCGGCCGC CTA ATG ATG ATG ATG ATG ATG
                          Not I. Stop Polyhistamine tag
                CTC TTC TAG TTG GCA
                Exon 10 CFTR, +15 to +132
                TGC
```

The following nucleotides were used to create the CFTR TARGET pre-mRNA mini gene (Exon 9+mini-Intron 9+Exon 10+5′ end Intron 10):

Forward CF18

(SEQ ID NO: 44)

```
                GACCT CTCGAG GGA TTT GGG GAA TTA TTT GAG
                            XhoI Exon 9 CFTR, 1 to 21.
Reverse CF19
```

(SEQ ID NO: 45)

```
                CTGACCT GCGGCCGC TAC AGT GTT GAA TGT GGT GC
                                NotI. Intron 9 5′ end.
Forward CF20
```

(SEQ ID NO: 46)

```
                CTGACCT GCGGCCGC CCA ACT ATC TGA ATC ATG TG
                                NotI. Intron 9 3′ end.
Reverse CF21
```

(SEQ D NO: 47)

```
                GACCT CTTAAG TAG ACT AAC CGA TTG AAT ATG
                    AflII Intron 10 5′ end.
```

The following oligonucleotides were used for detection of trans-spliced products:

Reverse Bio-His

(SEQ ID NO: 48)

```
                CTA ATG ATG ATG ATG ATG ATG
                Stop.Polyhistidine tag (5′ biotin label).
Reverse Bio-His(2)
```

(SEQ ID NO: 49)

```
                CGC CTA ATG ATG ATG ATG ATG
                3′ UT Stop.Polyhistidine tag (5′ biotin
                label).
Forward CF8
```

(SEQ ID NO: 50)

```
                CTT CTT GGT ACT CCT GTC CTG
                Exon 9 CFTR.
Forward CF18
```

(SEQ ID NO: 51)

```
                GACCT CTCGAG GGA TTT GGG GAA TTA TTT GAG
                            XhoI. Exon 9 CFTR.
Reverse CF28
```

(SEQ ID NO: 52)

```
                AAC TAG AAG GCA CAG TCG AGG
                Pc3.1 vector sequence (present in PTM 3′
                UT but not target).
```

Results

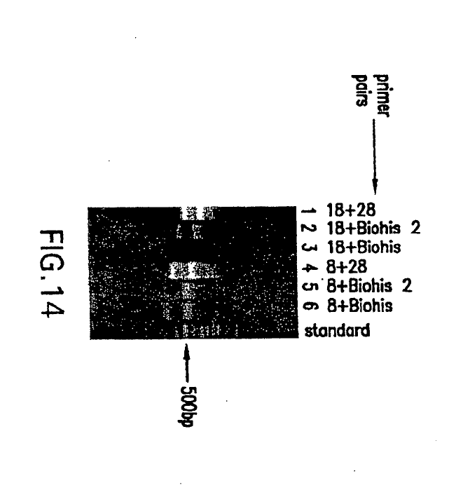

The PTM and target pre-mRNA were co-transfected in 293 embryonic kidney cells using lipofectamine (Life Technologies, Gaithersburg, Md.). Cells were harvested 24 h post transfection and RNA was isolated. Using PTM and target-specific primers in RT-PCR reactions, a trans-spliced product was detected in which mutant exon 10 of the target pre-mRNA was replaced by the wild type exon 10 of the PTM (FIG. 14). Sequence analysis of the trans-spliced product confirmed the restoration of the three nucleotide deletion and that splicing was accurate, occurring at the predicted splice sites (FIG. 15), demonstrating for the first time RNA repair of the cystic fibrosis gene, CFTR (Mansfield et al., 2000, Gene Therapy 7:1885-1895).

### EXAMPLE

### Double-Trans-Splicing

The following example demonstrates accurate replacement of an internal exon by a double-trans-splicing between a target pre-mRNA and a PTM RNA containing both 3′ and 5′ splice sites leading to production of full length functionally active protein.

As described herein, any pre-mRNA can be reprogrammed by providing a trans-reactive RNA molecule containing either a 3′-splice site, a 5′-splice site or both. The following example describes successful targeting and replacement of a single internal exon utilizing pre-trans-splicing molecules (PTMs) containing both the 5′ and 3′ splice sites. Such PTMs can promote two trans-splicing reactions with the intended target gene mediated by the splicesome(s). To test this mechanism, a splicing lacZ model target gene consisting of lacZ 5′ “exon”-CFTR mini-intron 9-CFTR exon 10 (ΔF508)-CFTR mini-intron 10 followed by lacZ 3′ “exon” was created. In this target transcript, a 124 bp central portion of the β-galactosidase ORF was substituted by exon 10 (ΔF508) of CFTR, thus it produces non-functional protein. A PTM consisting of the missing 124 bp lacZ “mini-exon” and a 5′ and 3′ trans-splicing domain containing binding domains (BDs) complementary to the target introns and exons was created. Transfection of HEK 293T cells with either target alone or PTM alone showed no detectable levels of β-gal activity. In contrast, 293T cells transfected with target plus PTM produced substantial levels of β-gal activity indicating the restoration of protein function. The accuracy of trans-splicing between the target and PTM was confirmed by sequencing the appropriate RT-PCR product, which revealed the predicted internal exon substitution. The feasibility of this approach in a disease model was tested by replacing the CFTR ΔF508 exon 10 with normal exon 10 containing F508 in cystic fibrosis. These results demonstrate that a trans-splicing technology can be easily adapted to correct many of the genetic defects whether they are associated with the 5′ exon or 3′ exon or any internal exon of the gene.

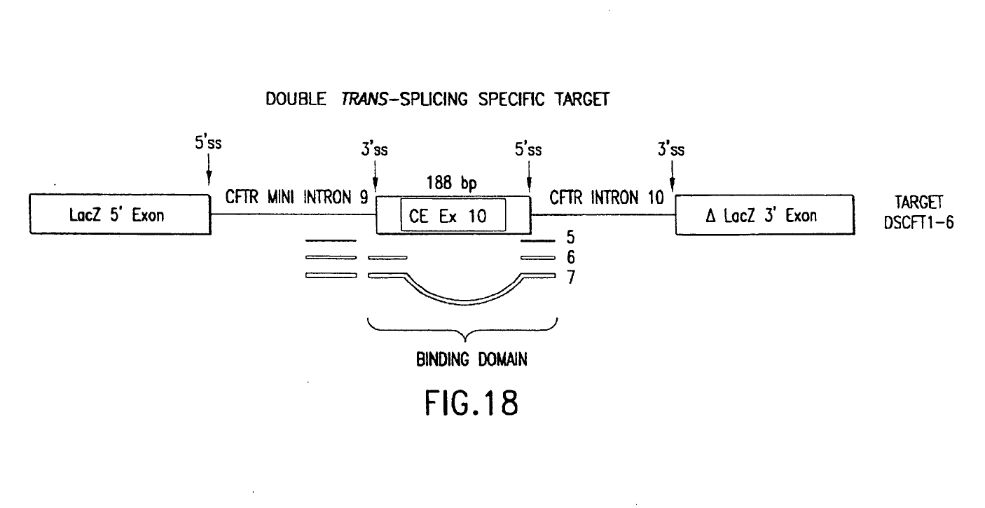

FIG. 18 is a schematic of a model lacZ target consisting of lacZ 5′ exon-CFTR mini-intron 9-CFTR exon 10 (delta 508)-CFTR min-intron 10 followed by the lacZ 3′ exon. In this target, a 124 bp central portion of the lacZ gene is substituted with CFTR exon 10 which has a mutation at position 508 (delta 508). The pre-mRNA target undergoes normal cis-splicing to produce an mRNA consisting of lacZ 5′ exon-CFTR exon 10 (delta 508) followed by the lacZ 3′ exon. Because of the disruption in β-galactosidase ORF it produces truncated proteins which are non-functional.

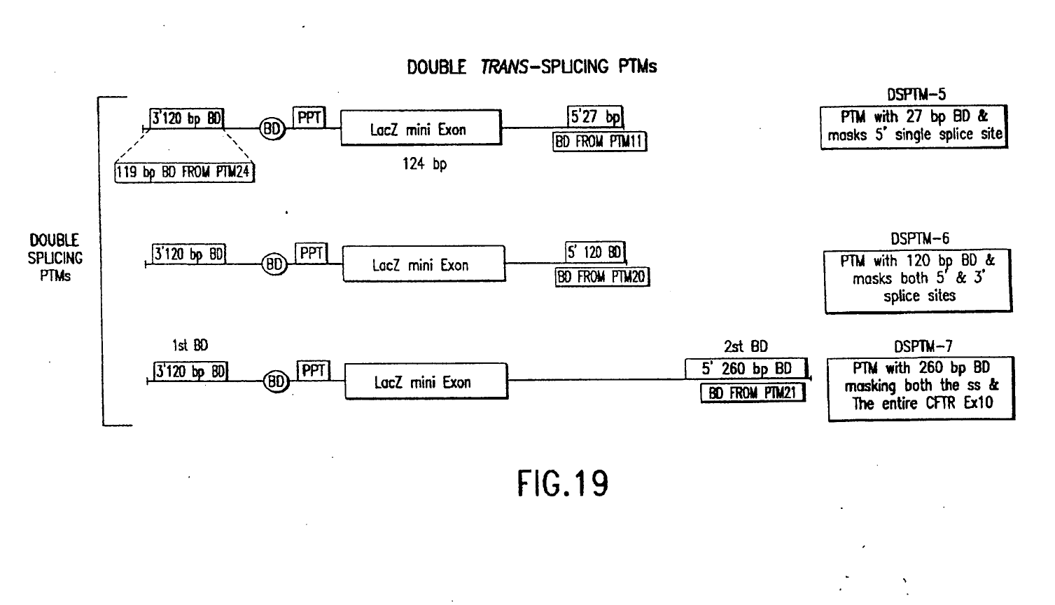

To restore β-gal function by double-trans-splicing, three PTMs were created consisting of the missing 124 bp lacZ “mini-exon” and a 5′ and 3′ trans-splicing domain containing binding domains complementary to the target introns and exons as shown in FIG. 19. These PTMs have an 120 bp 3′ binding domain (complementary to intron 9) from PTM24 (see below) used in 3′ exon replacement, spacer sequence, yeast branch point, polypyrimidine tract, 3′ acceptor AG dinucleotide, lacZ “mini-exon”, 5′ splice site, spacer sequence followed by the 5′ binding domain. These PTMs differ only in their 5′ binding domain sequences. DSPTM5 has a 27 bp BD which is complementary to intron 10 and blocks just the 5′ splice site of the target. DSPTM6 has 120 bp 5′ BD and covers both 5′ and 3′ splice sites of the target, while, DSPTM7 has 260 bp BD which masks both the splice sites (5′ and 3′) and also covers the entire exon of the target.

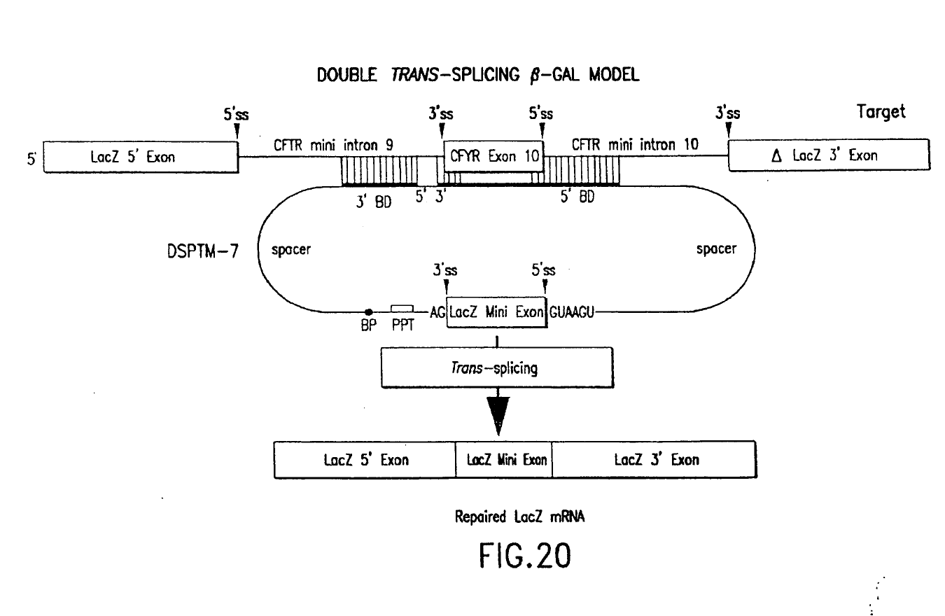

A schematic representation of a double-trans-splicing reaction showing the binding of DSPTM7 with DSCFT1.6 target pre-mRNA is shown in FIG. 20. 3′ BD: 120 bp binding domain complementary to mini-intron 9; 5′ BD (260 bp); second binding domain complementary to mini-intron 10 and exon 10. ss: splice sites; BP: branch point, and PPT: polypyrimidine tract.

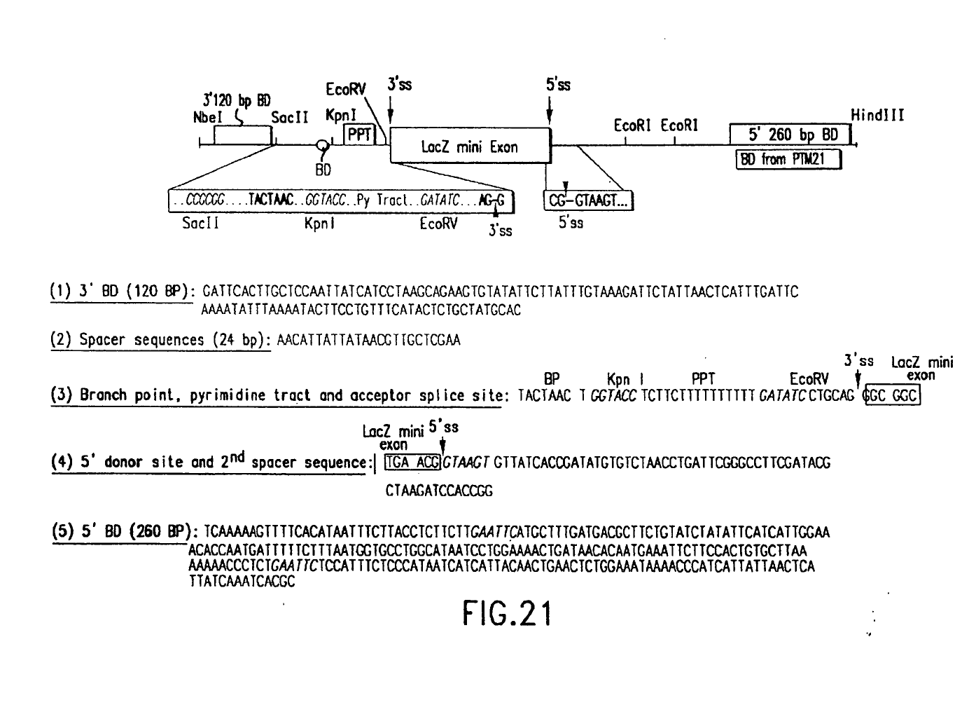

The important structural elements of DSPTM7 (FIG. 21) are as follows:

```
(1) 3′ BD (120 BP) (SEQ ID NO: 69):
        GATTCACTTGCTCCAATTATCATCCTAAGCAGAAGTGTATATTCTTATT
TGTAAAGATTCTATTAACTCATTTGATTCAAAATATTTAAAATACTTCCTGTTT
CATACTCTGCTATGCAC
(2) Spacer sequences (24 bp) (SEQ ID NO: 70):
AACATTATTATAACGTTGCTCGAA
(3) Branch point, pyrimidine tract and acceptor splice site(SEQ ID NO: 71):
                                                                                  3′ ss
      BP Kpn 1 PPT EcoRV ↓lacZ mini-exon
TACTAAC T GGTACC TCTTCTTTTTTTTTT GATATC CTGCAG |GGC GGC|
(4) 5′ donor site and 2^{nd} spacer sequence (SEQ ID NO: 72):
                          5′ ss
lacZ mini-exon ↓
|TGA ACG| GTAAGT GTTATCACCGATATGTGTCTAACCTGATTCGGGCCTTC
GATACGCTAAGATCCACCGG
(5) 5′ BD (260 BP)(SEQ ID NO: 73):
                          TCAAAAAGTTTTCACATAATTTCTTACCTCTTCTTGAATTCATGCTTTG
                ATGACGCTTCTGTATCTATATTCATCATTGGAAACACCAATGATTTTTCTTTAA
                TGGTGCCTGGCATAATCCTGGAAAACTGATAACACAATGAAATTCTTCCACT
                GTGCTTAAAAAAACCCTCTTGAATTCTCCATTTCTCCCATAATCATCATTACA
                ACTGAACTCTGGAAATAAAACCCATCATTATTAACTCATTATCAAATCACGC
```

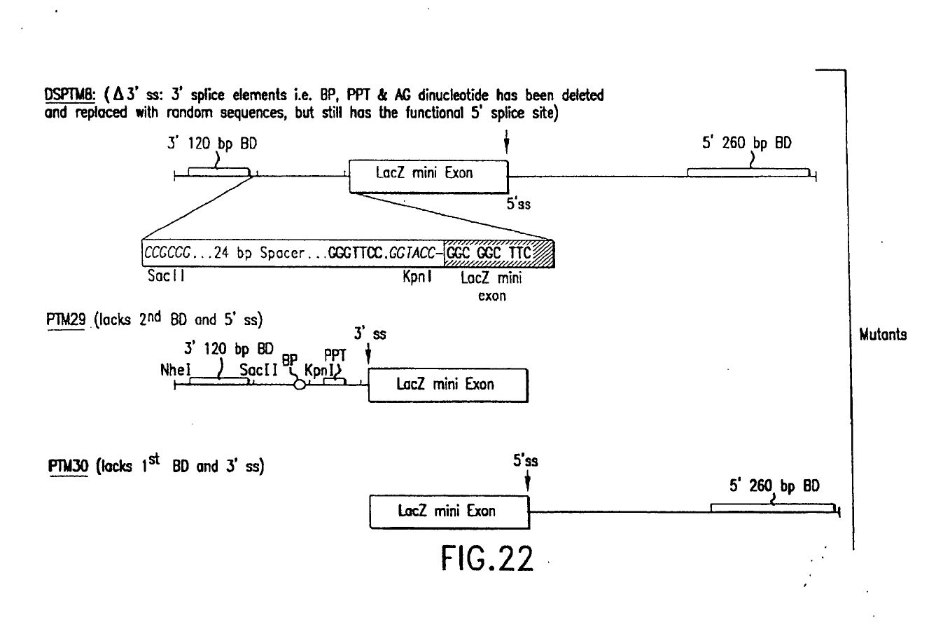

To determine whether the restoration of β-gal function is RNA trans-splicing mediated, the mutants are depicted in FIG. 22. DSPTM8 is a 3′ splice mutant in which the 3′ splice elements such as BP, polypyrimidine tract and the 3′ acceptor AG dinucleotides were deleted and replaced with random sequences (SEQ ID NO:85). This PTM still has 3′ and 5′ binding domains and the functional 5′ splice site. PTM29 lacks the 2^{nd} binding domain +5′ ss but still has the 3′ binding domain 3′ splice site, while PTM30 lacks the 1^{st} binding domain +3′ splice site but has the functional 5′ splice site and 2^{nd} binding domain.

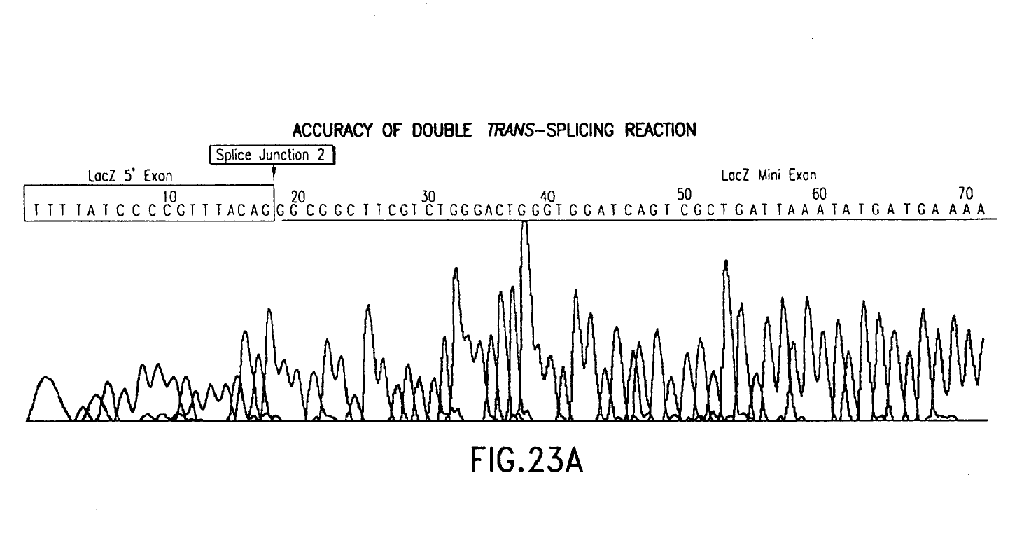

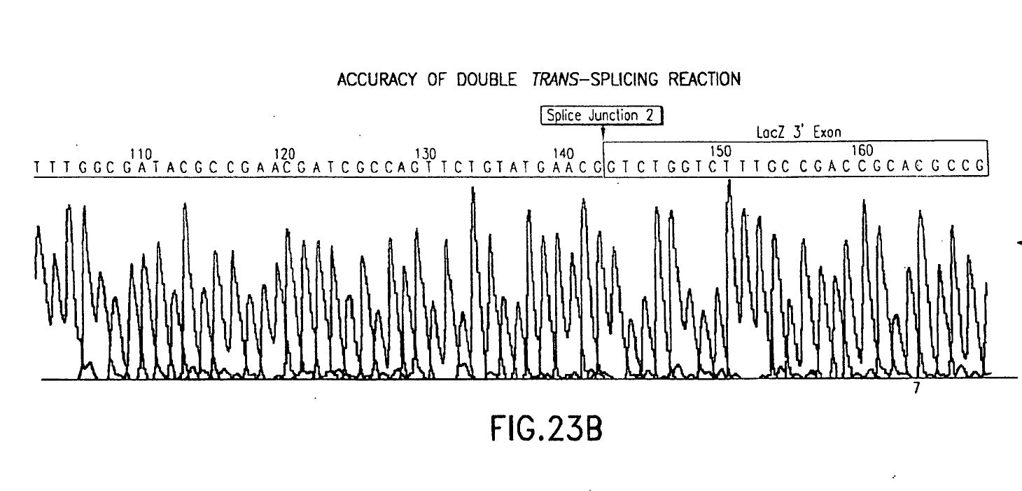

To examine the double-trans-splicing mediated restoration of β-gal function, 293T cells were either transfected with 2 Φg of target or PTM alone or co-transfected with 2 Φg of target +1.5 Φg of PTM using Lipofectamine Plus reagent. 48 hrs. after transfection, total RNA was isolated and analyzed by RT-PCR using K1-1F and Lac-6R primers. These primers amplify both cis- and trans-spliced products in a single reaction which were identified based on the size. The cis-spliced product is 295 bp in size while the trans-spliced product is 230 bp in size. To confirm that trans-splicing between DSPTM7 and DSCFT1.6 pre-mRNA is precise, RT-PCR amplified products were excised, re-amplified using K1-2F and Lac-6R primers and sequenced directly using K1-2F or Lac-6R primers. As shown in FIG. 23 trans-splicing occurred exactly at the predicted splice sites, confirming the precise internal exon substitution by two trans-splicing events (SEQ ID NO:86, 87).

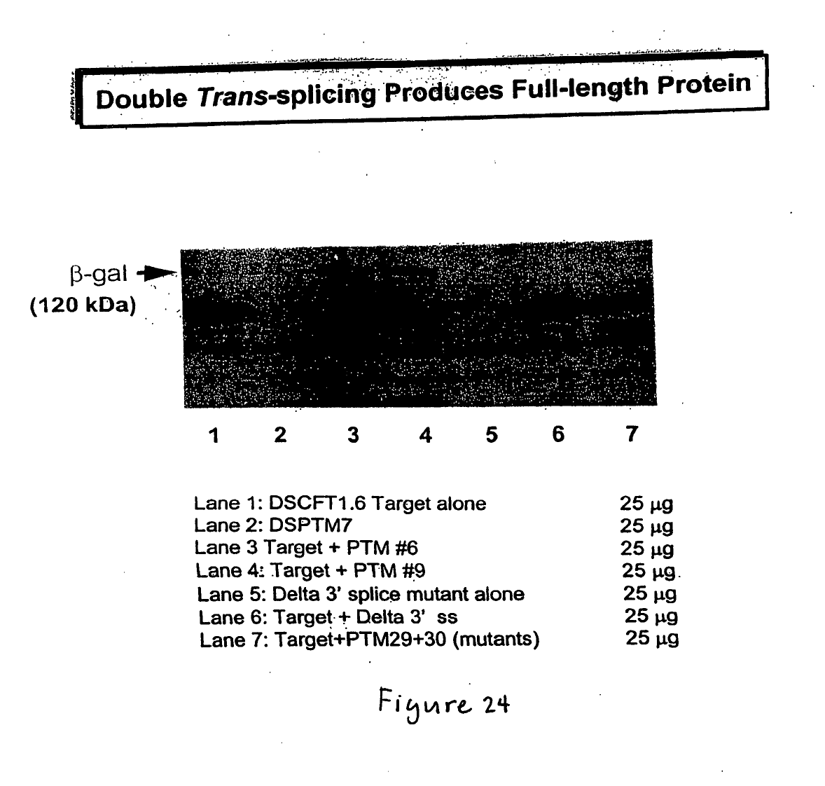

The repair of defective lacZ pre-mRNA by double trans-splicing events and subsequent production of full-length β-gal protein was investigated in co-transfection assays. 293T cells were co-transfected with DSCFT1.6 target and DSPTM7 expression plasmids, as well as with DSCFT1.6 target or DSPTM7 alone as controls. Western blot analysis of total cell lysates using polyclonal anti-β-galactosidase antiserum specifically recognized a −120 kDa protein only in cells co-transfected with DSCFT1.6 target+DSPTM7 plasmids (FIG. 24, lanes 3 and 4) but not in cells transfected with either DSCFT1.6 target (Lane 1) or DSPTM7 plasmid alone (Lane 2). Similarly, no full-length protein was detected in cells co-transfected with DSCFT1.6 target+3′ splice mutant (Lane 5 and 6) or PTM29 or 30 in which either 3′ trans-splicing domain or 5′ trans-splicing domains has been deleted (Lane 7). In addition, the 120 kDa protein band co-migrated with the full-length functional β-gal produced using lacZ-T1 plasmid (positive control, data not shown). These results not only confirmed the production of full-length protein by double-trans-splicing between the target and PTM but also demonstrated that both the 3′ splice site and 5′ splice sites are essential for this process.

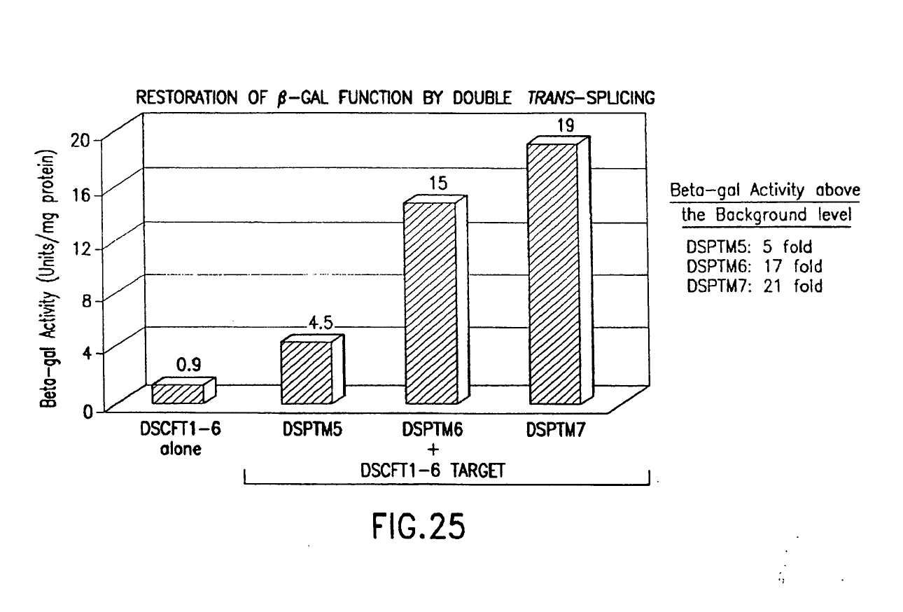

To determine whether the full-length protein produced by double-trans-splicing between the target pre-mRNA and DSPTM7 RNA is functionally active, 293T cells were co-transfected with DSCFT1.6 targeted+one of the double splicing PTMs 5, 6 or 7 expression plasmids, or transfected with DSCFT1.6 target or DSPTM7 alone. Total cell extracts were prepared and assayed for β-gal activity using ONPG assay (Invitrogen). β-gal activity in extracts prepared from cells transfected with either DSCFT1.6 target or DSPTM7 alone was almost identical to the background levels detected in mock transfection (FIG. 25). In contrast, 293T cells co-transfected with DSCFT1.6 target and DSPTM7 produced −21 fold higher levels of β-gal activity over the background (FIG. 25). These results confirmed the accurate double-trans-splicing between the target pre-mRNA and PTM RNA and production of the full-length functional protein.

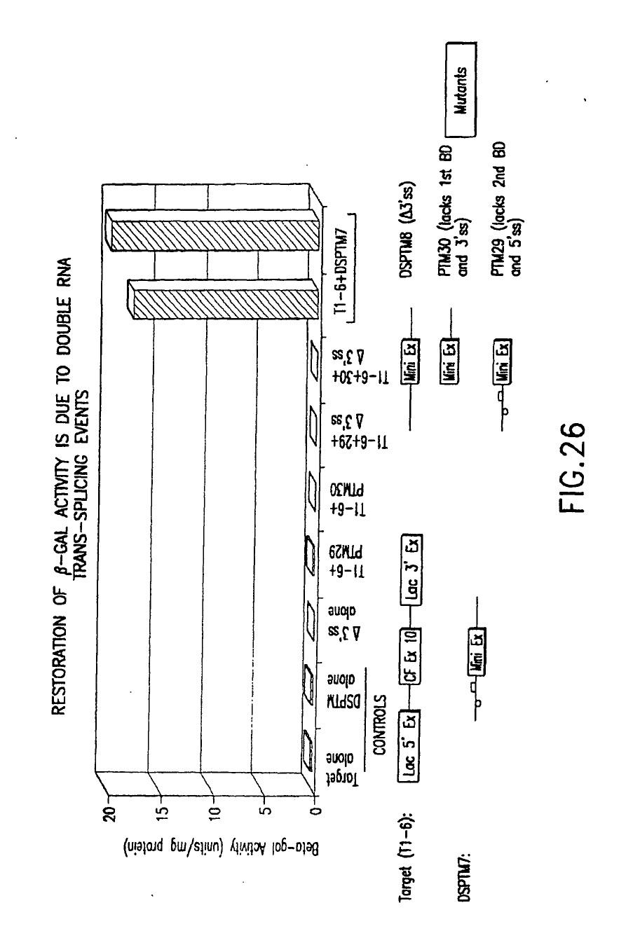

To confirm that restoration of β-gal activity by double-trans-splicing reaction is absolutely depended on the presence of both 3′ and 5′ splice sites of the PTM, we constructed several mutants: (a) DSPTM8, is identical to DSPTM7 except the functional 3′ spice elements (branch point, polypyrimidine tract and the 3′ acceptor AG dinucleotides) were deleted and substituted with random sequences (see FIG. 22 for details); (b) PTM29 lacks 5′ splice site as well as the 5′ binding domain but has the 3′ binding domain and 3′ splice site, and (c) PTM30 lacks 3′ binding domain and 3′ splice site but has the 5′ splice site and 5′ binding domain. β-gal activity in extracts prepared from cells transfected with either DSCFT1.6 target or DSPTM7 alone was almost identical to the background levels detected in mock transfection (FIG. 26). Similarly, no significant increase in β-gal activity was detected in cells transfected with either DSPTM8 alone (3′ splice site mutant) or co-transfection of DSCFT1.6 target+one of the above mutant PTMs. On the other hand, cells co-transfected with DSCFT1.6 target and DSPTM7 with functional 3′ and 5′ splice sites produced substantial levels of β-gal activity over the background (FIG. 26). These results confirmed the requirement of both splice sites in the double-splicing PTM and also eliminated the possibility that restoration of β-gal activity was due to complementation between the truncated proteins (FIG. 26).

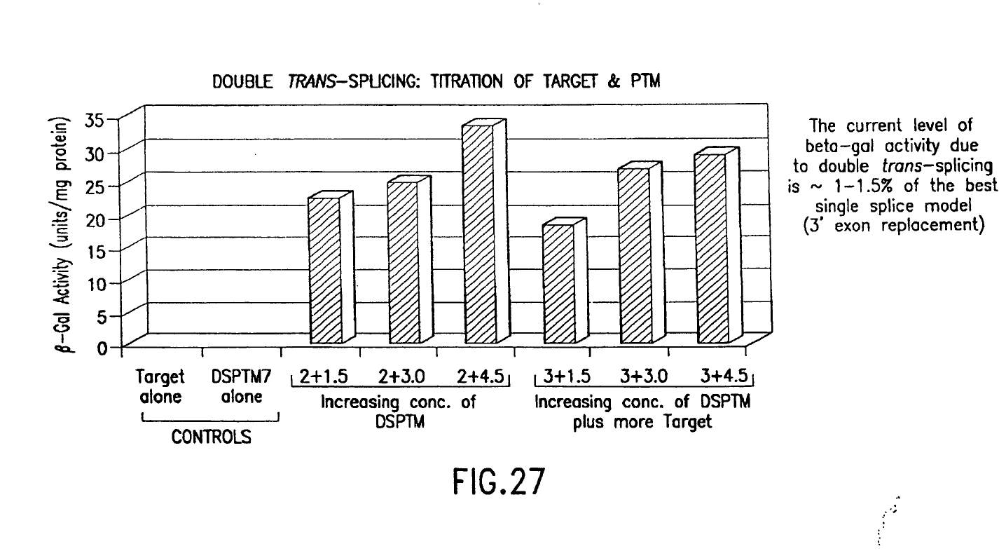

Different concentrations of the target and PTM were co-transfected and analyzed for β-gal activity restoration. As expected, 293T cells co-transfected with DSCFT1.6 target+DSPTM7 showed substantial levels of β-gal activity (−30 fold) over the controls. Increasing the concentrations of the PTM by 2 and 3 fold did increase the level of β-gal activity, but not significantly (FIG. 27). These results further confirmed the double-trans-splicing mediated restoration of β-gal enzyme function.

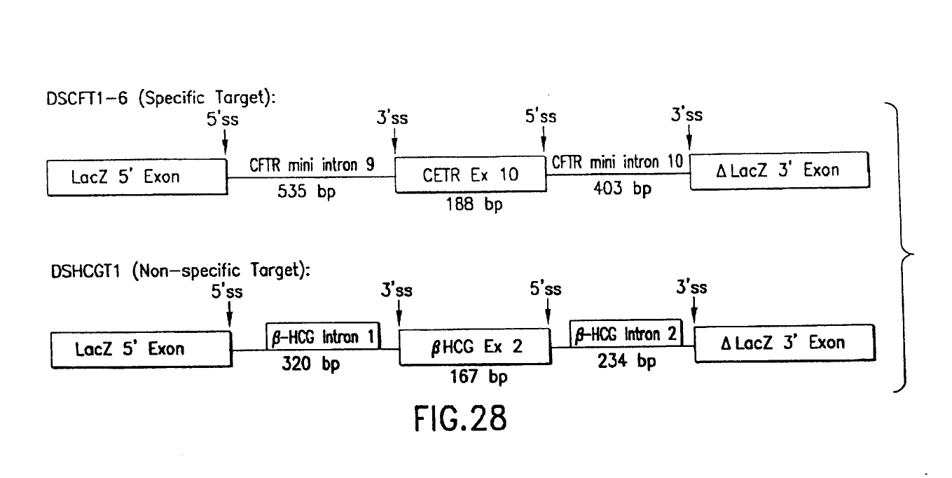

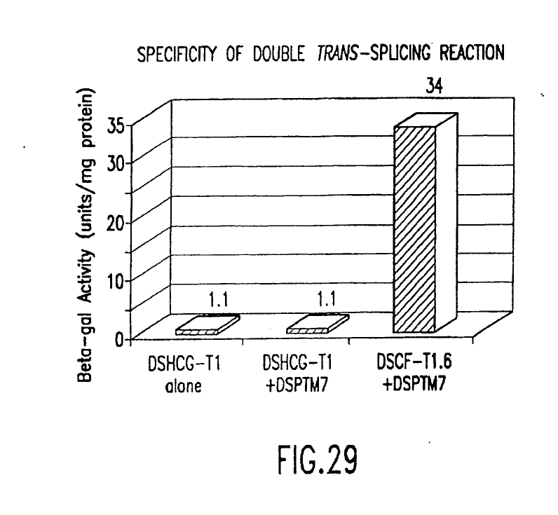

The specificity of double-trans-splicing reaction was examined by constructing a non-specific target (DSHCGT1.1) which is similar to that of specific target (DSCFT1.6) but has βHCG intron 1-βHCG exon 2 and βHCG intron 2 instead of CFTR mini-intron 9-CFTR exon 10 (delta 508) and CFTR mini-intron 10 (FIG. 28). RT-PCR analysis of the total RNA isolated from cells transfected with either DSHCGT1.1 (non-specific target) alone or in combination DSPTM7 (targeted to DSCFT1.6 target) failed to produce the expected 314 bp double-trans-spliced product. On the other hand, RT-PCR analysis of the total RNA prepared from cells co-transfected with specific target+PTM produced the expected 314 pb product. This was further confirmed by β-gal activity assay of the total cellular extract. The level β-gal activity detected in cells transfected with non-specific target alone or in combination with DSPTM7 targeted to DSCFT1.6 target was almost identical to the background level. In contrast substantial levels of β-gal activity was detected in cells co-transfected with specific target (DSCFT1.6)+DSPTM7 (FIG. 27). These results confirmed that the double-trans-splicing is highly specific.

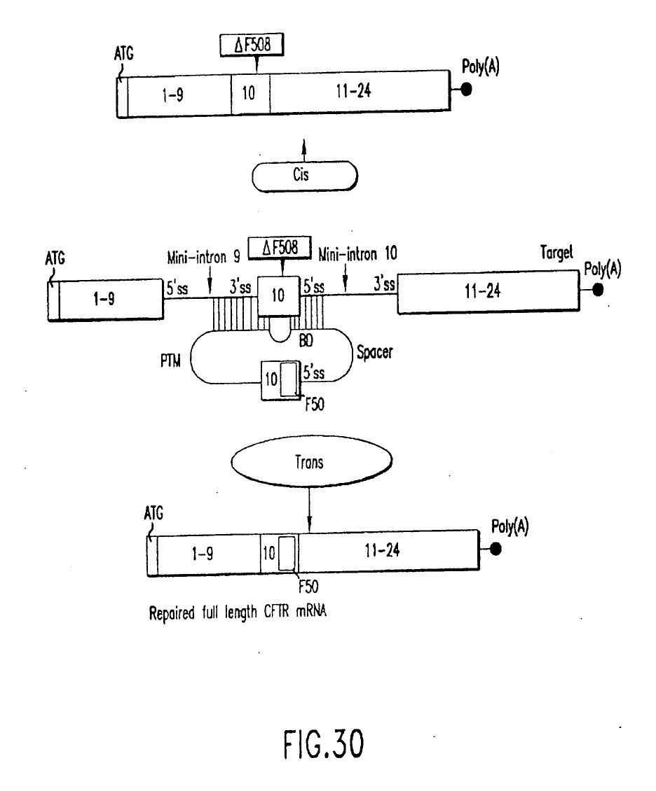

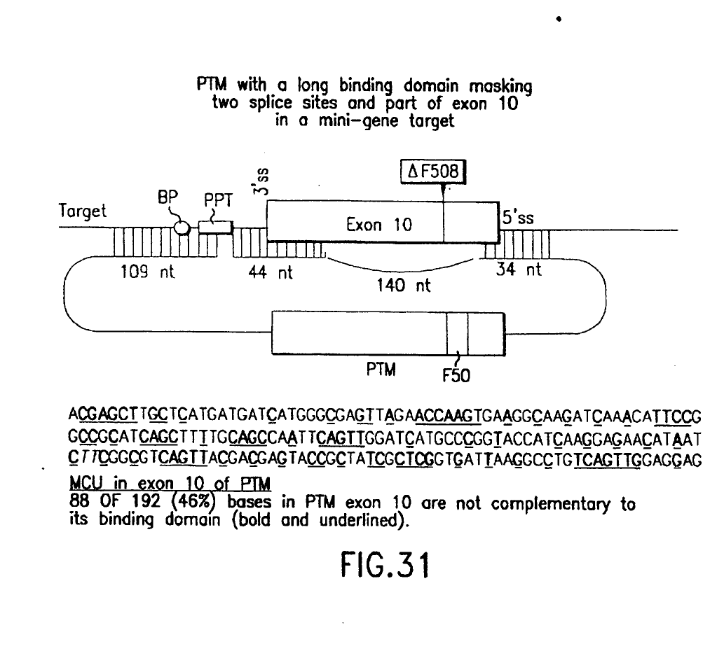

The repair model in FIG. 30 shows a portion of a target CFTR pre-mRNA consisting of exons 1-9, mini-intron 9, exon 10 containing the delta 508 mutation, mini-intron 10 and exons 11-24 (FIG. 30). The PTM shown in the figure consists of exon 10 coding sequences (containing codon 508) and two trans-splicing domains each with its own splicing elements (acceptor and donor sites, branchpoint and pyrimidine tract) and a binding domain complementary to intron 9 splice site, part of exon 10 (5′ and 3′ ends) and intron 10 5′ splice site (SEQ ID NO:88)(FIG. 31 (DS-CF1)). Exon 10 of the PTM also has modified codon usage throughout to reduce antisense effects between exon 10 of the PTM and it's own binding domains and for PTMs that have binding domains which are complementary to exon sequences (FIG. 31). A double-trans-splicing event between the PTM and target should produce a repaired full-length mRNA.

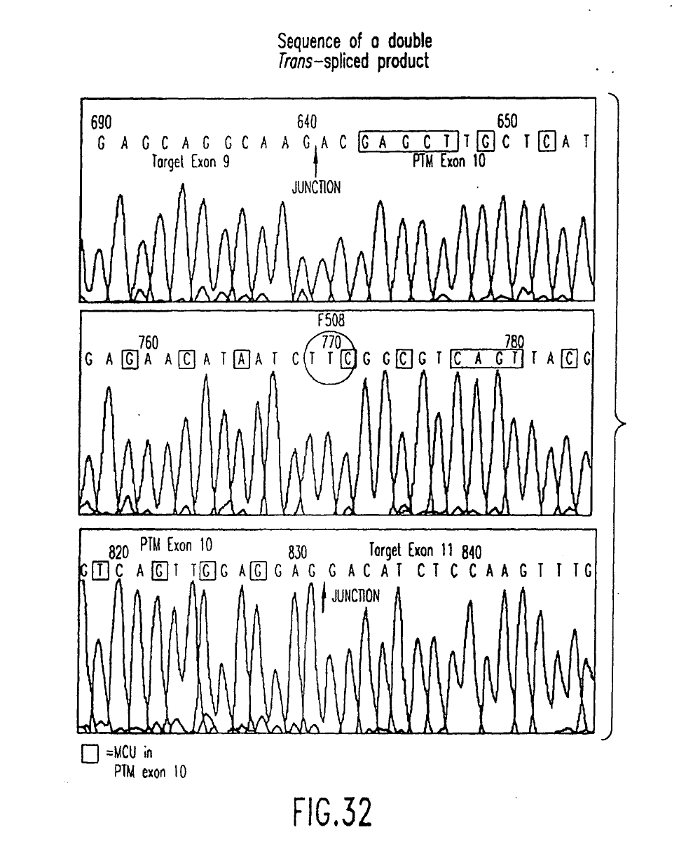

FIG. 32 shows the sequence of a single PCR product showing target exon 9 correctly spliced to PTM 20 exon 10 (with modified codons) (upper panel) (SEQ ID NO:89), codon 508 in exon 10 of the PTM (middle panel) (SEQ ID NO:90) and PTM exon 10 correctly spliced to target exon 11 (lower panel) (SEQ ID NO:91). The sequence of a repaired target was generated by RT-PCR followed by PCR.

### EXAMPLE

### Trans-Splicing Repair of the Cystic Fibrosis Gene Using a PTM that can Perform 5′ Exon Replacement

The key advantage of using 5′ exon replacement for gene repair are

(a) it permits replacement of the 5′ portion of a gene

(b) the construct requires less sequence and space than a full-length gene construct,

(c) PTMs can be produced that lack a polyA signal which should prevent PTM translation, and (d) the 5′ end can be modified to increase translation.

Materials and Methods

Plasmid Construction

The CFTR coding sequences (exons 1-10) for PTM30 were generated by PCR using a partial cDNA plasmid template (61160; American Type Culture Collection, Manassas, Va.). The trans-splicing domain (TSD) [including the binding domain, spacer sequence, polypyrimidine tract (PPT), branchpoint (BP) and 3′ splice site] was generated from a PCR product (using an existing plasmid template) and by annealing oligonucleotides. The different fragments (the TSD and coding sequences) were then cloned into pcDNA3.1(−) using appropriate restriction sites. Oligodeoxynucleotide primers were procured from Sigma Genosys (The Woodlands, Tex.). All PCR products were generated with either REDTaq (Sigma, St. Louis, Mo.), or cloned Pfu (Stratagene, La Jolla, Calif.) DNA Polymerase. PCR primers for amplification contained restriction sites for directed cloning. PCR products were digested with the appropriate restriction enzymes and cloned into the mammalian expression plasmid pc3.1DNA(−) (Invitrogen, Carlsbad, Calif.).

Cell Culture and Transfections

Constructs were cotransfected in human embryonic kidney (HEK) 293T or 293 cells (1.25×10⁶ cells per 60 mm poly-d-lysine coated dish) using LipofectaminePlus (Life Technologies, Gaithersburg, Md.) and the cells were harvested 48 h after the start of transfection. Total RNA was isolated as described in the manufacturers instructions (Epicenter Technologies, Inc.). HEK 293T cells were grown in Dulbecco's Modified Eagle's Medium (Life Technologies) supplemented with 10% v/v fetal bovine serum (Hyclone, Inc., Logan, Utah). All cells were kept in a humidified incubator at 37 EC and 5% CO₂.

Reverse Transcription-Polymerase Chain Reaction (RT-PCR)

RT-PCR was performed using an EZ-RT-PCR kit (Perkin-Elmer, Foster, Calif.). Each reaction contained 0.03 to 1.0 Φg of total RNA and 80 ng of a 5′ and 3′ specific primer in a 40 Φl reaction volume. RT-PCR products were electrophoresed on 2% Seaken agarose gels. The PTM- and target-specific oligonucleotides used to generate trans-spliced products are 5′-CGCTGGAAAAACGAGCTTGTTG-3′ (primer CF93) (SEQ ID NO:74) and 5′-ACTCAGTGTGATTCCACCTTCTC-3′ (primer CF111) (SEQ ID NO:75), respectively. The PTM- and target-specific oligonucleotides used to generate cis-spliced products were CF1 and CF93. The sequence of oligonucleotide CF1 is 5′-GACCTCTGCAGACTTCACTTCTAATGATGATTATGG-3′ (SEQ ID NO:76).

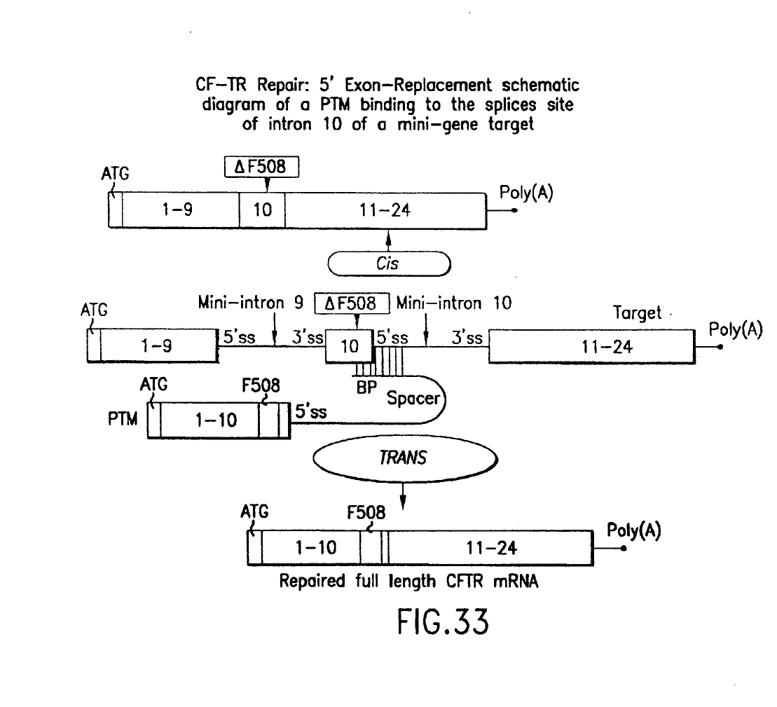

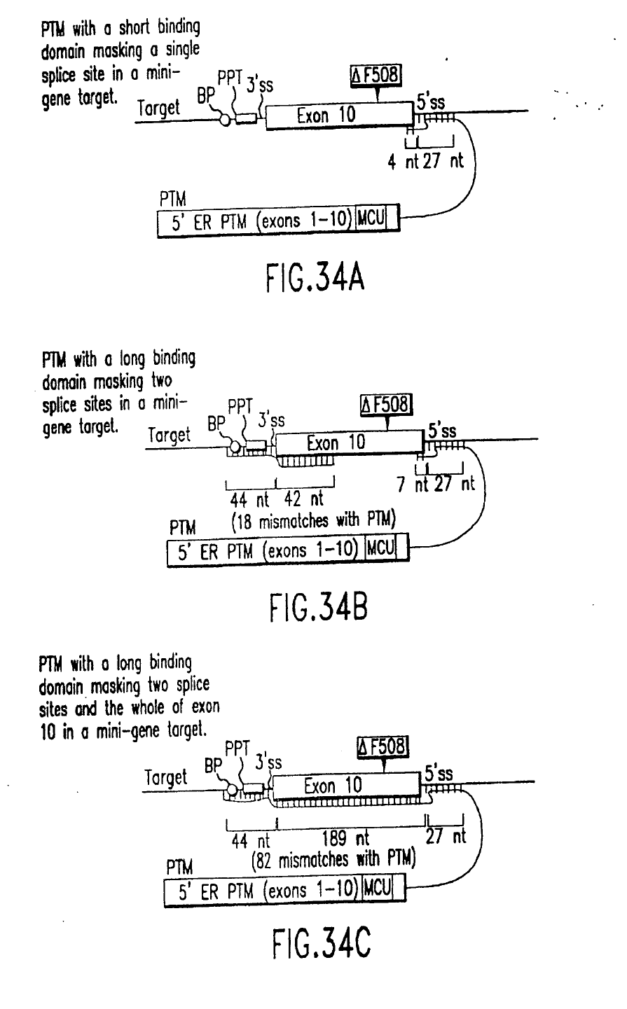

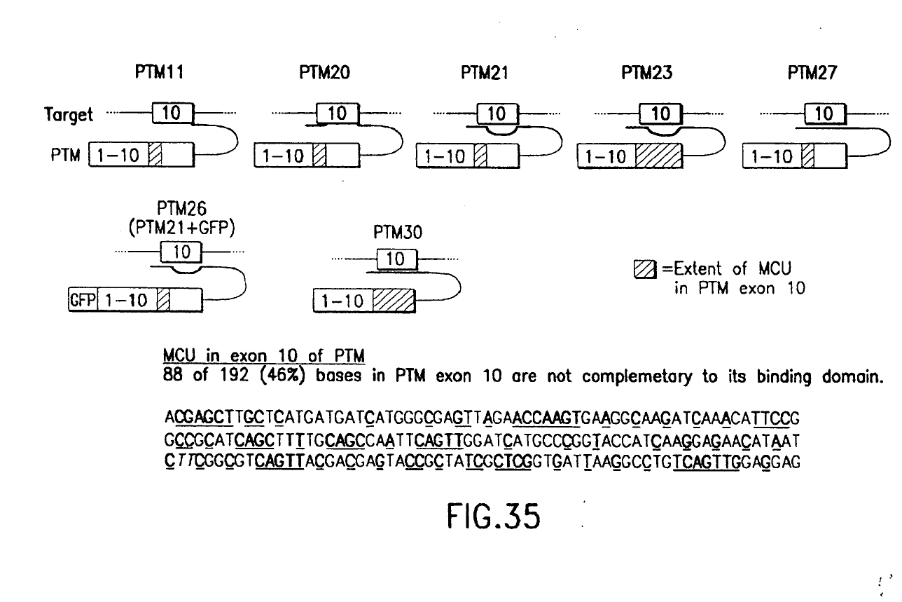

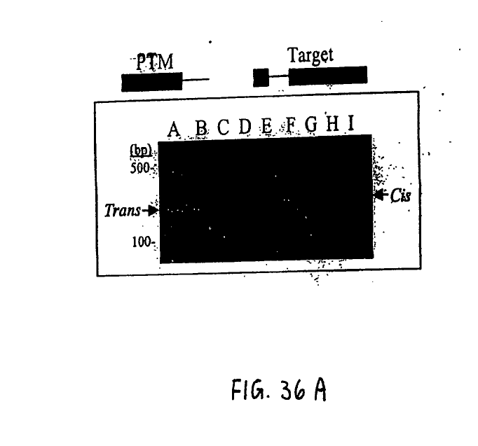

The repair model in FIG. 33 shows a portion of a target CFTR pre-mRNA consisting of exons 1-9, mini-intron 9, exon 10 containing the delta 508 mutation, mini-intron 10 and exons 11-24 (FIG. 33). The PTM shown in the figure consists of exon 1-10 coding sequences (containing codon 508) and a trans-splicing domain with its own splicing elements (donor site, branchpoint and pyrimidine tract) and a binding domain. Several PTMs have been constructed with different binding domains. Three examples are shown in FIG. 34. In FIG. 34A the binding domain is complementary to the splice site of intron 9 and part of exon 10 (3′ end; CF-PTM 11). In FIG. 34B the PTM has an extended binding domain which also covers the 5′ end of exon 10 and the 3′ splice site of intron 9 (CF-PTM 20). In the last example (FIG. 34C) the binding domain is the same as that shown in panel B except the binding domain extends the full-length of exon 10 (CF-PTM 30). In the latter case the PTM exon 10 has modified codon usage to reduce antisense effects with it's own binding domain (FIG. 34). Further examples of binding domains are shown in FIG. 35.

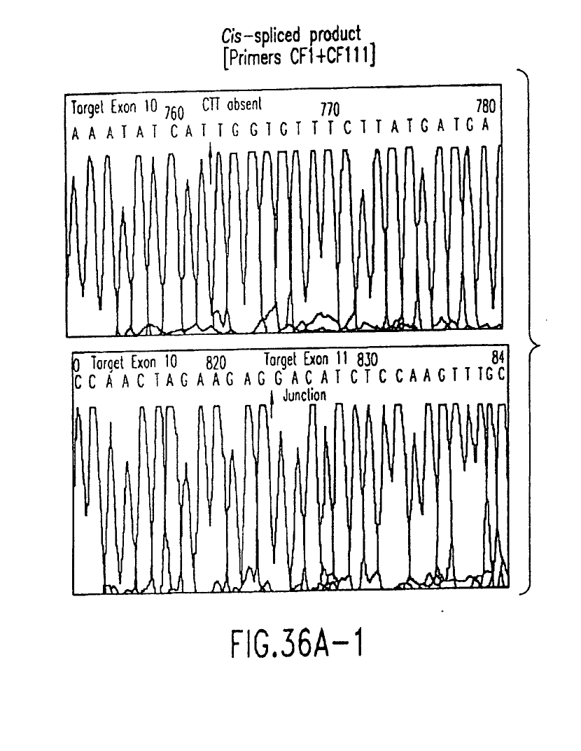

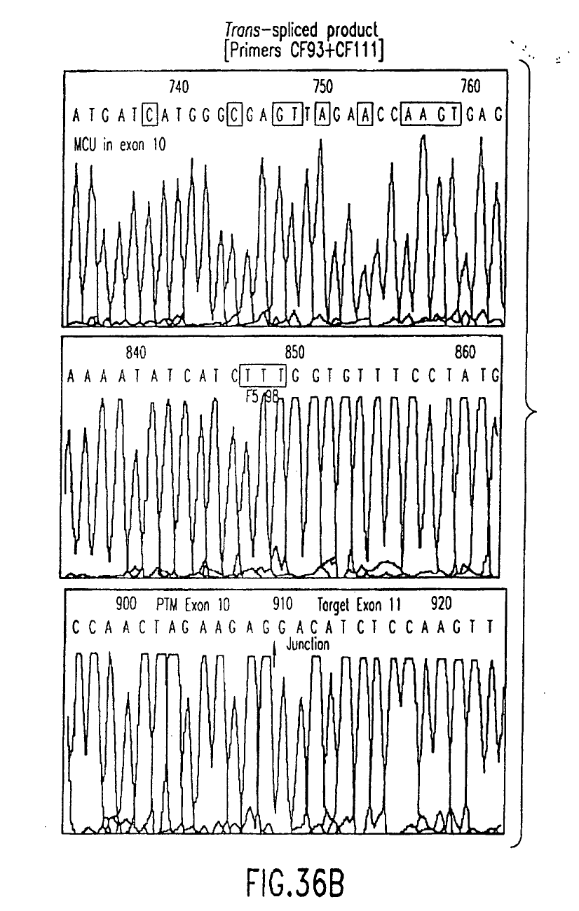

FIG. 36 shows the sequence of cis- and trans-spliced products. The top panel of FIG. 36A shows target exon 10 with it's three missing nucleotides (CTT) (SEQ ID NO:93), whilst the lower panel shows exon 10 and 11 of the target correctly spliced together (SEQ ID NO:94).

FIG. 36B is a partial sequence of a single PCR product showing the modified codons in exon 10 of the PTM (upper panel) (SEQ ID NO:95), codon 508 in exon 10 of the PTM (middle panel) (SEQ ID NO:96), and PTM exon 10 correctly spliced to target exon 11 (lower panel) (SEQ ID NO:97), indicating that trans-splicing is accurate. The sequence of the repaired target was generated by RT-PCR followed by PCR.

### EXAMPLE

### PTMs with a Long Binding Domain, which may be Discontinuous, Have Increased Trans-Splicing Efficiency and Specificity

Materials and Methods

Cell Culture

Human embryonic kidney cells (293 or 293T) were from the University of North Carolina tissue culture facility at Chapel Hill (Chapel Hill, N.C.). Cells were maintained at 37 EC in a humidified incubator with 5% CO₂ in Dulbecco's modified Eagle's medium (Life Technologies, Bethesda, Md.) supplemented with 10% v/v fetal bovine serum (Hyclone, Logan, Utah). Cells were passaged every 2-3 days using 0.5% trypsin and re-plated at the desired density. Stable cells, expressing an endogenous mutant lacZ pre-mRNA (lacZCF9) were maintained in the presence of 0.5 mg/ml G418 (Calbiochem, San Diego, Calif.).

Recombinant Plasmids

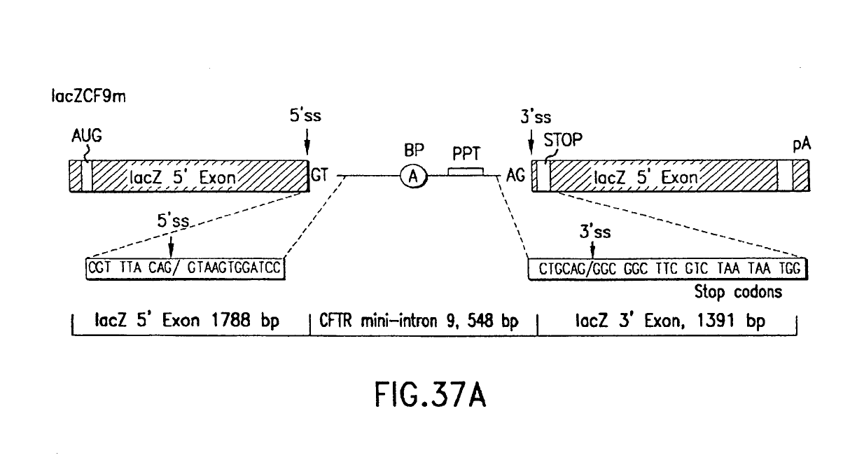

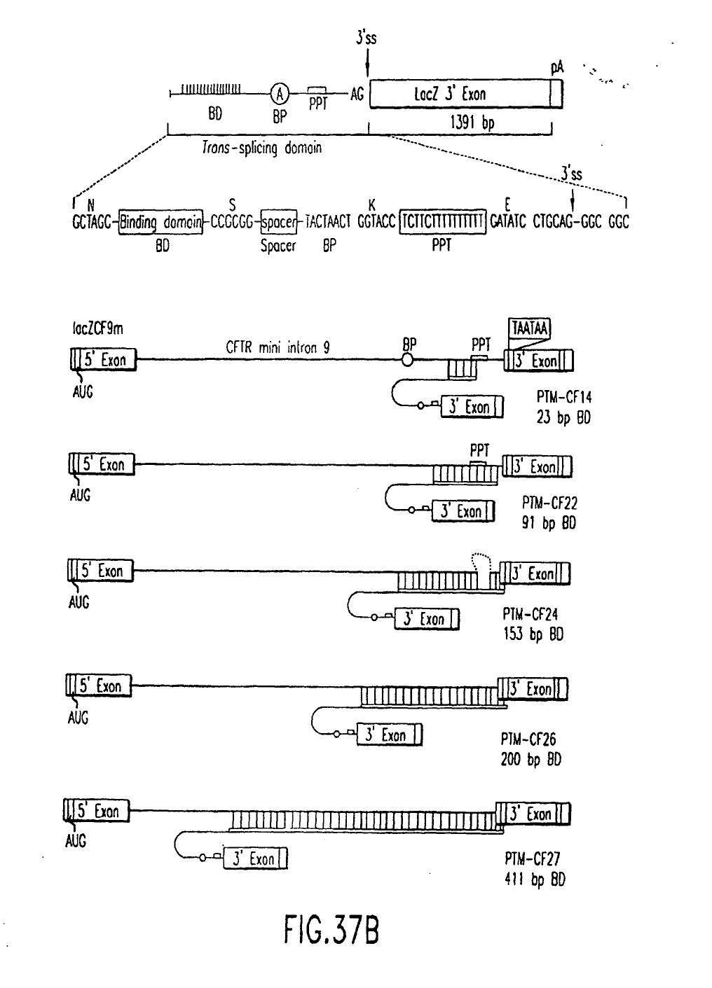

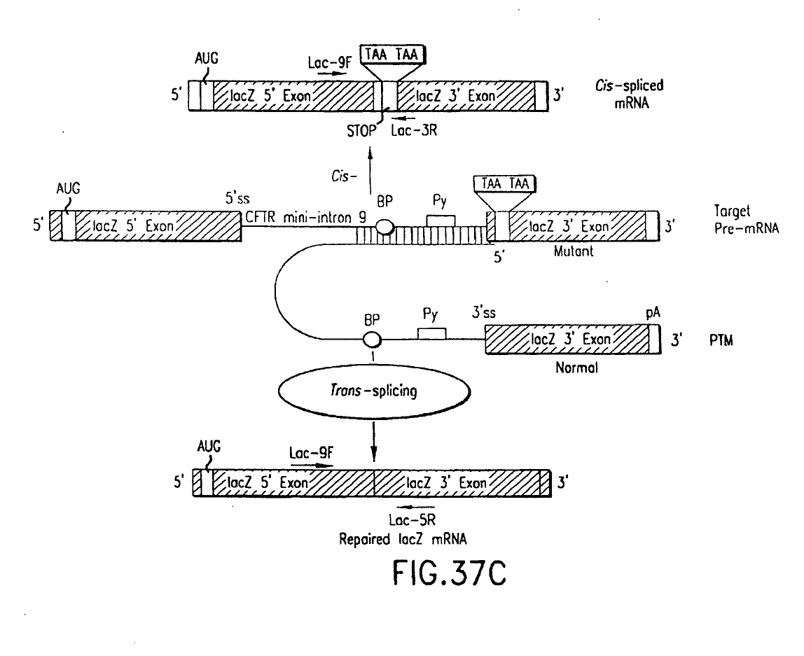

Targets: pc3.1lacZCF9, pc3.1lacZCF9m, and pc3.1lacZHCG1m. pc3.1lacZCF9 encodes for a normal lacZ pre-mRNA was constructed using lacZ coding sequences nucleotides 1-1788 as 5′ exon, CFTR mini-intron 9 followed by lacZ coding sequences nucleotides 1789-3174 as 3′ exon. This is similar to pc3.1lacZ-T2 construct but without stop codons in the lacZ 3′ exon and has CFTR mini-intron 9 instead of βHCG6 intron 1 (FIG. 37A). CFTR mini-intron 9 was PCR amplified using plasmid T5 as template and primers CFIN-9F (5′-CTAGGATCCCGTTCTTTTGTTCTTCACT ATTAA) (SEQ ID NO:77) and CFIN-9R (5′-CTAGGGTTACCGAAGTAAAACCATACTTATTAG, restriction sites underlined) (SEQ ID NO:78), digested with BamH I and BstE II and cloned in place of BHCG6 intron 1 of pc3.1lacZ-T2 plasmid. pc3.1 lacZCF9m expresses a defective lacZ pre-mRNA and is identical to pc3.1lacZCF9 but contains two in-frame non-sense codons in the 3′ exon (FIG. 37A). pc3.1lacZHCG1m is a chimeric target, which includes the lacZ 5′ exon followed by intron 1 and exon 2 of βHCG6. This is similar to pc3.1lacZCF9m except that it contains exon 2 of βHCG6 in place of mutant lacZ 3′ exon. βHCG6 exon 2 was PCR amplified using βHCG6 plasmid (accession # X00266) as template DNA and primers HCGEx-2F (5′-GCATGGTTACCCTGCAGGGGCTGCTGCTGTTGCTG) (SEQ ID NO:79) and HCGEx-2R (5′-CTGAAAGCTTGTTAACCAGCTCACCATGGTGGGGCAG, restriction sites underlined) (SEQ ID NO:80) digested with BstE II and Hind III and cloned in place of the lacZ 3′ exon of pc3.1lacZCF9m. Plasmid pcDNA3.1/HisB/lacZ (Invitrogen, Carlsbad, Calif.) was used as DNA template to produce 5′ and 3′ lacZ exons. The lacZ 5′ exon is 1788 bp long, has an ATG initiation codon, lacZ 3′ exon (without stop codons) is 1385 bp long and has a transcription termination signal at the end of the 3′ exon. CFTR mini-intron 9 and βHCG6 intron 1 are 548 bp and 352 bp in size, respectively, and both have 5′ and 3′ splice signals. Exon 2 of βHCG6 is 162 bp long and has a transcription termination signal at the end of the exon.

Pre-trans-splicing Molecules (PTMs): PTM-CF14 is an identical version of pcPTM1 with minor modifications in the trans-splicing domain (FIG. 37B). PTM-CF14 is a linear version and contains a 23 bp antisense binding domain (BD) (5′-ACCCATCATTATTAGGTCATTAT) (SEQ ID NO:81) complementary to CFTR mini-intron 9, 18 bp spacer, a canonical branch point sequence (UACUAAC; BP) and an extended polypyrimidine tract (PPT) followed by normal lacZ 3′ exon. PTM-CF22, PTM-CF24, PTM-CF26 and PTM-CF27 are identical to PTM-CF14 except they differ in length of the BD (FIG. 37B). sPTM-CF18 has a 32 bp BD, sPTM-CF22 and sPTM-CF24 contain the same BD as PTM-CF22 and PTM-CF24, respectively. In these PTMs, the binding domains were modified to create intra-molecular stem-loop structure (“safety”) to mask the 3′ splice-site of the PTM. Different binding domains were produced by PCR amplification using specific primers (with unique Nhe I and Sac II sites) and a plasmid containing CFTR mini-intron 9 as template. PCR products were digested with Nhe I and Sac II and cloned into a PTM plasmid consisting of spacer sequences, 3′ splice elements (BP, PPT and acceptor AG dinucleotide) followed by a normal lacZ 3′ exon.

Transfection of Plasmid DNAs into 293T Cells

The day before transfection, 1×10⁶ 293T cells were plated on 60 mm plates coated with Poly-D-lysine (Sigma, St. Louis, Mo.) to enhance the adherence of cells and grown for 24 hr at 37 EC. Cells were transfected with expression plasmids using LipofectaminePlus reagent according to standard protocols (Life Technologies, Bethesda, Md.). In a typical co-transfection, 2 Φg of pc3.1lacZCF9m target and 1.5 Φg of PTM expression plasmids were transfected into cells and for controls (target and PTM alone transfections) total DNA concentration was normalized to 3.5 (Dg with pcDNA3.1 vector.

Forty-eight hours after transfection the plates were rinsed with PBS, cells harvested and total RNA or DNA was isolated using MasterPure RNA/DNA purification kit (Epicenter Technologies, Madison, Wis.). Contaminating DNA in the RNA preparation was removed by treating with DNase I, while, contaminating RNA in the DNA preparation was removed by digesting with RNase A at 37 EC for 30-45 min.

Reverse Transcription-Polymerase Chain Reaction (RT-PCR)

RT-PCR was performed as suggested by manufacturer using an EZ rTth RNA PCR kit (Perkins-Elmer, Foster City, Calif.). A typical reaction (50 Φl) contained 25-500 ng of total RNA, 100 ng of 5′ target specific primer (common to cis- and trans-spliced products) (Lac-9F, 5′-GATCAAATCTGTCGATCCTTCC) (SEQ ID NO:82) and 100 ng of 3′ primer (Lac-3R, 5′-CTGATCCACCCAGTCCCATTA, target specific primer for cis-splicing (SEQ ID NO:83), and Lac-5R, 5′-GACTGATCCACCCAGTCCCAGA (SEQ ID NO:84), PTM specific primer for trans-splicing), IX reverse transcription buffer (100 mM Tris-HCl, pH 8.3, 900 mM KCL with 1 mM MnCl₂), 200 ΦM dNTPs and 10 units of rTth DNA polymerase. RT reactions were performed at 60 EC for 45 min. followed by 30 sec pre-heating at 94 EC and 25-35 cycles of PCR amplification at 94 EC for 18 sec, annealing and extension at 60 EC for 1 min followed by a final extension at 70 EC for 7 min. The reaction products were analyzed by agarose gel electrophoresis.

Protein Preparation and β-Gal Assay

Total cellular protein from cells transfected with expression plasmids was isolated by freeze thaw method and assayed for β-galactosidase activity using a β-gal assay kit (Invitrogen, Carlsbad, Calif.). Protein concentration was measured by the dye-binding assay using Bio-Rad protein assay reagents (BIO-RAD, Hercules, Calif.).

Western Blot

About 5-25 Φg of total protein was electrophoresed on a 7.5% SDS-PAGE gel and electroblotted onto PVDF-P membrane (Millipore). After blocking for 1 hr at room temperature (blocking buffer: 5% dry milk and 0.1% Tween-20 in 1×PBS), the blot was incubated with a 1:2500 dilution of polyclonal rabbit anti-β-galactosidase antibody for 1 hr at room temperature (Research Diagnostics Inc. NJ), washed 3× with blocking buffer and then incubated with a 1:5000 diluted anti-rabbit HRP conjugated secondary antibody. After incubating at room temperature for 1 hr, it was washed 3× in blocking buffer and developed using ECLPlus Western blotting reagents (Amersham Pharmacia Biotech, Piscataway, N.J.).

In Situ β-Gal Staining

Cells were monitored for the expression of functional β-galactosidase using a β-gal staining kit (Invitrogen, Carlsbad, Calif.). The percentage of β-gal positive cells were determined by counting stained vs. unstained cells in 5-10 randomly selected fields.

Selection of Neomycin Resistant Clones Expressing an Endogenous Defective lacZ Pre-mRNA Target

On day 1, 1×10⁶ 293 cells were plated on 60 mm plates and grown for 24 hr at 37 EC. On day 2, the cells were transfected with 2 Φg of pc3.1lacZCF9m using LipofectaminePlus transfection reagent as described above. 48 hr post-transfection, cells were split (1:20 ratio) and grown in media containing 0.5 mg/ml G418. At the end of 2 weeks, neomycin resistant colonies were selected, pooled, expanded and maintained constantly in the presence of G418.

Results

A model system was developed that permits facile and versatile analysis of spliceosome mediated RNA trans-splicing in cells. The bacterial lacZ gene was split with a truncated intron 9 from the Cystic Fibrosis Transmembrane Conductance Regulator (CFTR) gene (FIG. 37A). This split lacZ gene, when introduced into human 293T cells, directed the synthesis of a lacZ pre-mRNA that could splice properly. The open reading frame of the lacZ gene was mutated by insertion of two in-frame nonsense codons near the 5′ end of the second exon (FIG. 37A). This lacZ gene is referred to as lacZCF9m. In 293T cells, lacZCF9m directs the synthesis of lacZCF9m pre-mRNA, which encodes a truncated β-galactosidase (β-gal) protein that does not have enzymatic activity. Cells bearing the lacZCF9m gene are a model system for genetic disorders caused by loss of function mutations.

Pre-trans-splicing molecules (PTMs) were designed to trans-splice with lacZCF9m pre-mRNA and repair the mutation caused by the two nonsense codons. PTMs were constructed with binding domains spanning 23, 91 and 153 nucleotides (nt), which we named PTM-CF14, PTM-CF22 and PTM-CF24 (FIG. 37B). The PTM-CF24 binding domain does not bind 153 contiguous nt in the targeted CFTR gene intron 9, but rather creates a loop of 47 nt in the target in between two regions of complementary of 27 and 126 nt (FIG. 37B). These PTMs were predicted to repair the deficiency created by lacZCF9m (FIG. 37C).

Semi-quantitative RT-PCR analysis was used to tests the efficiency of trans-splicing mediated by PTMs with long target binding domains. Repair of lacZCF9m transcripts by trans-splicing was tested in two different ways: co-transfection of PTM and target (lacZCF9m) plasmids or transfection of cells that had been modified to express the target as an endogenous pre-mRNA. Co-transfecting plasmids encoding PTMs with the lacZCF9m plasmid provided a facile method for screening the former for efficiency. PTM-CF22 and PTM-CF24 were approximately 3-fold and 10-fold more efficient than PTM-CF14 in a semi-quantitative RT-PCR assay suggesting a significant improvement in mRNA repair (FIG. 38). Sequencing of the RT-PCR products showed that trans-splicing was accurate, resulting in proper ligation of the exons from the target and the PTM. Moreover, mutation of key cis-acting elements in the 3′ splice site of the PTMs resulted in an abrogation of trans-splicing. In these and all other assays described herein controls were carried out to rule out recombination at the DNA level. Thus, repair of the lacZCF9m transcripts was a result of targeted RNA trans-splicing.

Transfection of PTM-CF14, -CF22 or -CF24 into 293 cells bearing an endogenous lacZCF9m gene confirmed that the longer target binding domains provided the PTMs with higher efficiency (FIG. 38B). It should be noted that similar levels of RT-PCR trans-splicing specific product were obtained after 30 PCR cycles and 35 cycles for PTM-CF24 and PTM-CF14, respectively. The data therefore suggests that PTMs with long binding domains repaired lacZCF9m transcripts at least an order of magnitude better than previously described PTMs.

More than one in ten transcripts of lacZCF9m can be repaired by trans-splicing. Quantitative, real-time PCR was used to measure the fraction of lacZCF9m transcripts repaired by PTMs with long binding domains. The co-transfection assay described above was used in these experiments. PTM-CF14, which contains a binding domain of 23 nt, was shown to repair between 1.2 and 1.6% of lacZCF9m RNAs in 293T cells and 2.1% of lacZCF9m RNAs in the H1299 human lung cancer cells. PTM-CF24, which has a 153 not long binding domain, was significantly more efficient, correcting between 12.1 and 15.2% of lacZCF9m RNAs in 293T cells and 19.7% in H1299 cells. This in effect resulted in a measurable reduction in the levels of lacZCF9m mRNA. These data also confirmed the remarkable capability of this RT-PCR assay to distinguish between the products of cis-splicing, the lacZCF9m and mRNA, and the products of trans-splicing, repaired lacZCF9m mRNA. This is the first true quantification of the efficacy of trans-splicing mediated mRNA repair at the RNA level. These data confirm the suggestions of the semi-quantitative RT-PCR analysis shown above. Similar experiments were carried out using 293 cells that express an endogenous lacZCF9m pre-mRNA target. Consistent with the data shown above, PTM-CF24 was ten times more efficient than PTM-CF14, with the former correcting between 1.3 and 4.1% of endogenous lacZCF9m transcripts. These data confirmed that increasing the length of the PTMs provided a remarkable enhancement in trans-splicing efficiency.

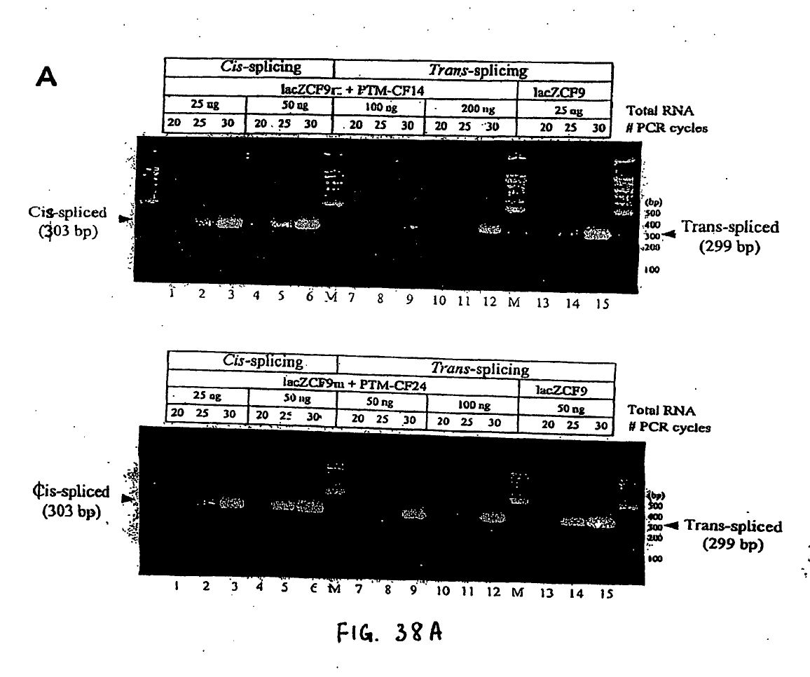

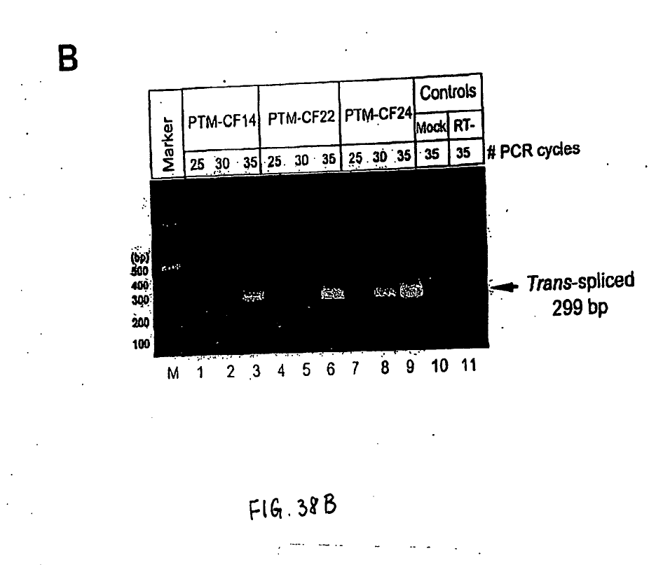

Trans-splicing mediated mRNA repair results in the synthesis of active β-galactosidase. At the cellular level, the ultimate criterion for the success of mRNA repair is the production of an active protein. Using a western assay it was determined that full-length β-gal was produced as a result of trans-splicing. Full-length β-gal was not observed following transfection of 293T cells with plasmids encoding lacZCF9m or PTM-CF24. Co-transfection of both plasmids, however, resulted in robust production of full-length β-gal protein, which was readily detectable using anti-β-gal antiserum (FIG. 39). This result complements enzymatic activity data suggests that the latter was not due to a complementation by truncated β-gal proteins. The Western blot analysis revealed that full-length β-gal protein was made in 293T cells by trans-splicing and furthermore confirmed that the PTMs with long binding domains were efficiently spliced.

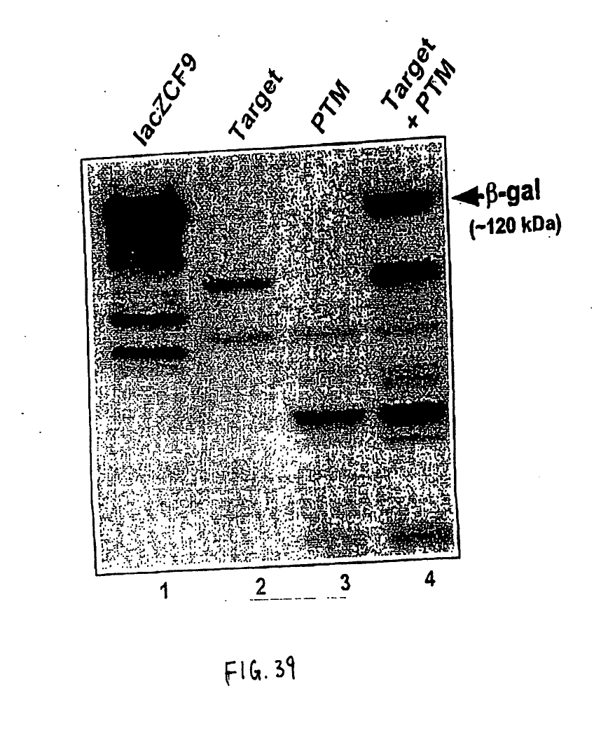

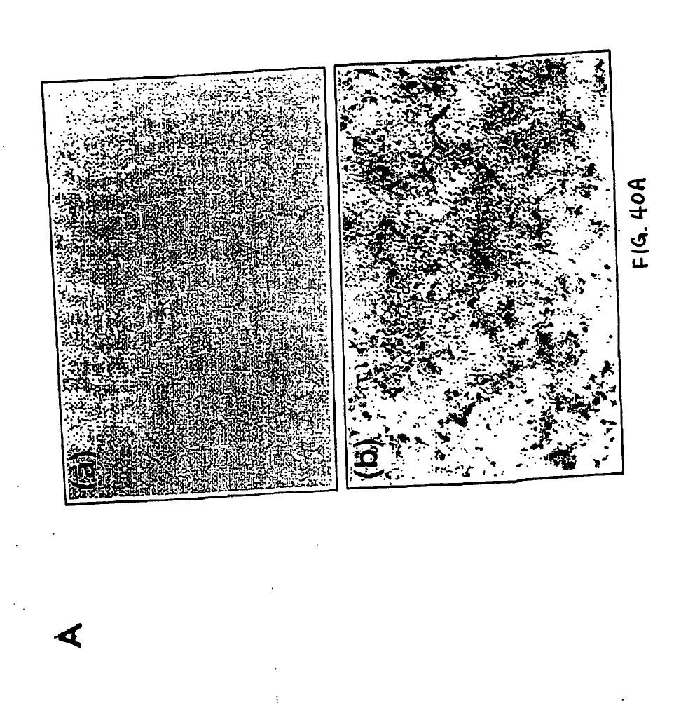

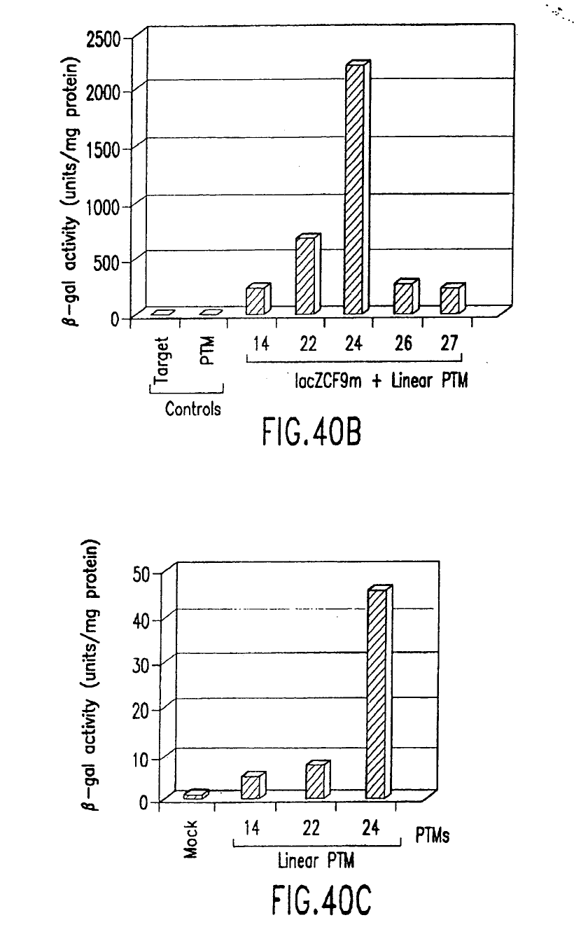

Appropriate repair of β-gal mRNA and synthesis of full-length β-gal protein should lead to the production of active enzyme. Indeed, 293T cells co-transfected with lacZCF9m and PTM-CF24 were shown to have β-gal activity measured either in situ (FIG. 40A) or in extracts (FIG. 40B). This activity was shown to depend on the trans-splicing between the target pre-mRNA and the PTM. The quantitative in solution assay further confirmed the data presented above: PTM-CF22 and PTM-CF24 were 2.9 and 9.3 fold more efficient respectively than PTM-CF14. Most impressive, however, were results using 293 cells that harbor lacZCF9m as a stable endogenous gene. When these cells were transfected with PTM-CF14 the levels of β-gal activity obtained were barely above background. Transfection with PTM-CF24, however, resulted in a considerable level of β-gal activity (FIG. 40C). This was paralleled by the appearance of full-length β-gal protein. These data demonstrate a sizeable increase in the efficiency of trans-splicing to repair a mutated pre-mRNA. In fact all prior reports of repair of endogenous RNA in mammalian cells by either group I ribozymes or trans-splicing have been only documented using RT-PCR, an indication of the low level of repair.

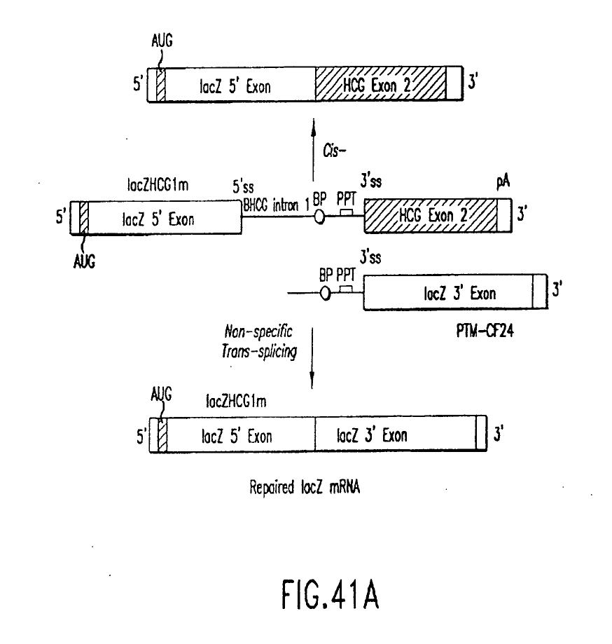

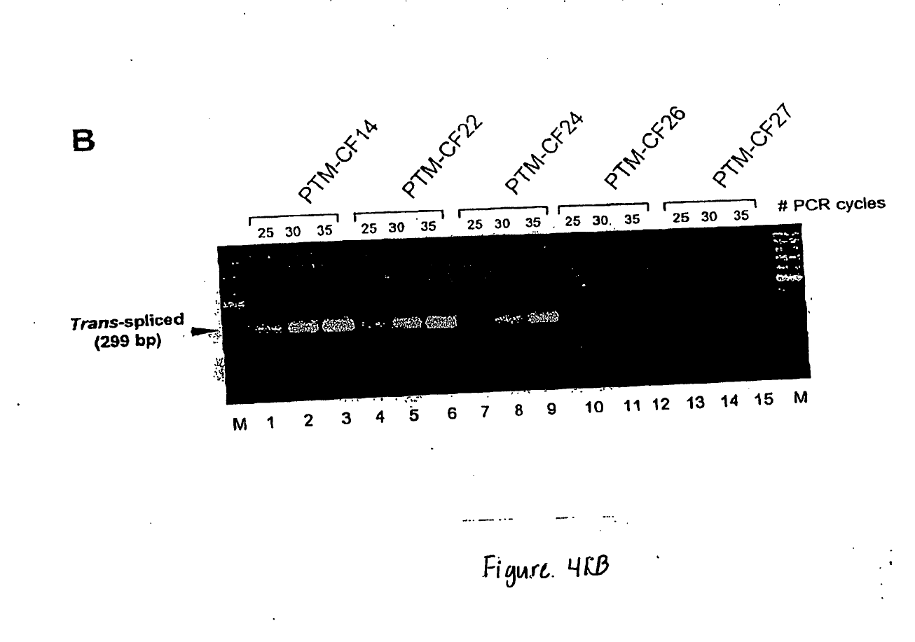

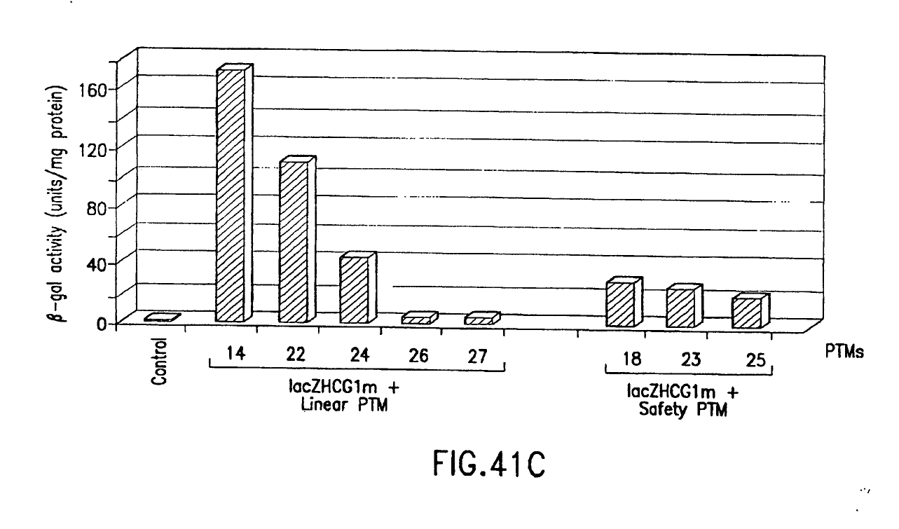

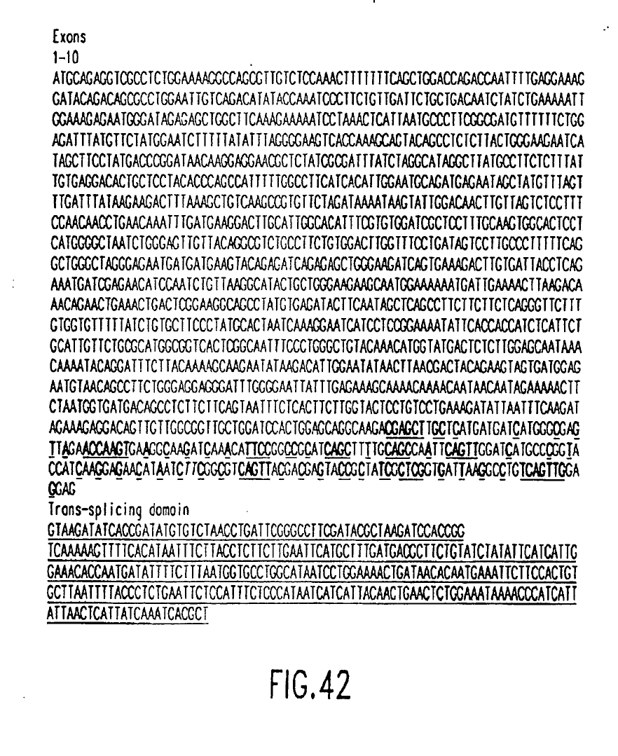

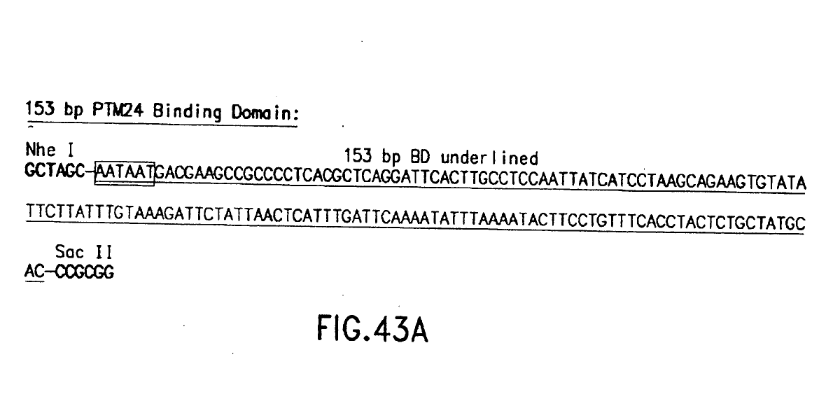

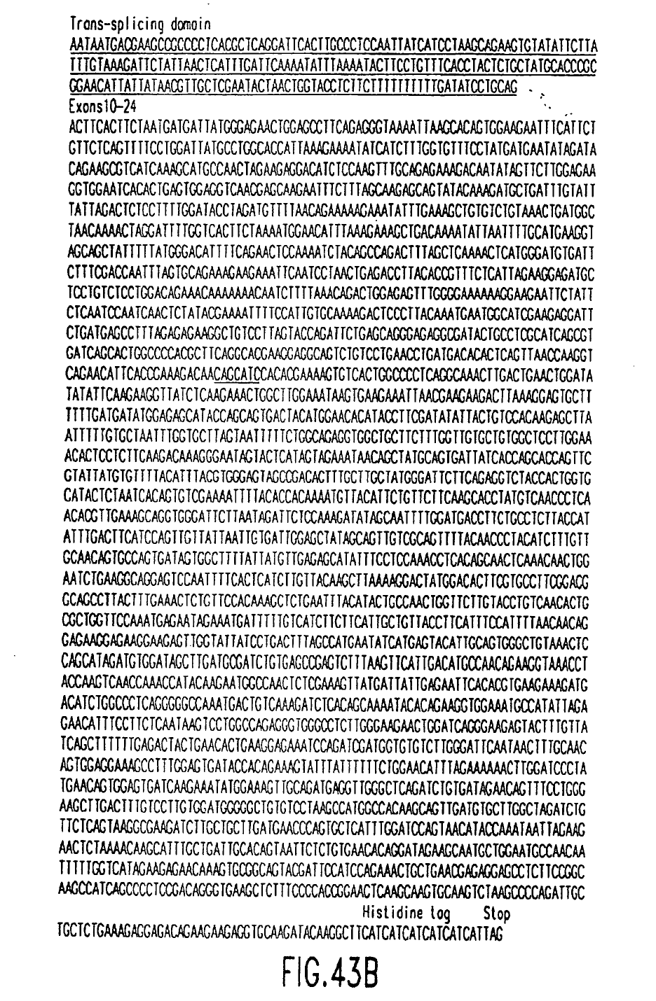

PTMs with very long binding domains are highly specific. It was shown that a secondary structure within the binding domain could enhance specificity of PTMs in HeLa nuclear extracts. In order to ascertain the specificity of the trans-splicing reactions in vivo a second target gene was prepared, which could serve as reporter of non-specific reactions. This gene, which is referred to as lacZHCG1m, shares the first exon with lacZCF9m. The intron in lacZHCG1m is intron 1 of the β-subunit of the human chorionic gonadotropin gene 6 (βhCG6) and the second exon is exon 2 of the same gene. lacZHCG1m drives the synthesis of a pre-mRNA that is spliced correctly to yield a chimeric mRNA that does not encode a full-length β-gal (see below). PTM-CF14, -CF22 and -CF24 are not targeted to lacZHCG1m pre-mRNA since there is no complementarity between the binding domains in these PTMs and the target gene. Any trans-splicing between these PTMs and lacZHCG1m pre-mRNA is therefore non-specific (FIG. 41A).

293T cells were transfected with PTM-CF14, -CF22 or -CF24 and the level of non-specific trans-splicing was determined by RT-PCR and by in solution β-gal assays. Semi-quantitative RT-PCR suggested that PTM-CF24 was significantly less likely than PTM-CF14 to trans-splice with lacZHCG1m pre-mRNA. Measurement of β-gal activity confirmed this; cells co-transfected with lacZHCG1m and PTM-CF24 produced 3.7 fold less β-gal than those co-transfected with lacZHCG1m and PTM-CF14 (FIG. 41C). Based on these data it was estimated that PTM-CF24 is 50 times more likely to trans-splice to its target than to a non-specific target. A “safety” version of PTM-CF24, sPTM-CF24, did not confer further specificity (FIG. 41C). Nonetheless, for PTMs with shorter binding domains a “safety” stem involving the binding domain was seen to improve specificity in vivo (FIG. 41C). It was concluded from these data that the longer binding domains resulted in PTMs that were not only more efficient but also more specific.

The observation that long binding domains increased the specificity of PTMs suggested that very long binding domains (>200 nt) could further enhance discrimination. Plasmids encoding PTM-CF26 and -CF27, which have binding domains that span 200 nt and 411 nt respectively, were constructed and co-transfected with lacZHCG1m plasmid. Non-specific trans-splicing of these two PTMs was barely detectable with RT-PCR (FIG. 41B). As measured by the β-gal assay PTM-CF26 and -CF27 had minimal non-specific trans-splicing activity (FIG. 41C). In a specific trans-splicing reaction with lacZCF9m as measured by the solution β-gal assay PTM-CF26 was as active as PTM-CF14 (FIG. 41B). It was estimated that PTM-CF26 is 80 times more likely to trans-splice to the specific target (lacZCF9m) than to a non-specific target (lacZHCG1m). Therefore, inclusion of very long binding domains confers to these PTMs very high specificity.

### EXAMPLE

### Correction of the Factor VIII Gene Using 3′ Exon Replacement

Hemophilia is a bleeding disorder caused by a deficiency in one of the blood clotting factors. Hemophilia A, which accounts for about 80 percent of all cases is a deficiency in clotting factor VIII. The following section describes the successful repair of the clotting factor VIII gene using spliceosome mediated trans-splicing and demonstrates the feasibility of repairing the factor VIII using gene therapy.

The coding region for mouse factor VIII PTM (exons 16-24) was PCR amplified from a cDNA plasmid template using primers that included unique restriction sites for directed cloning. All PCR products were generated with cloned Pfu DNA Polymerase (Stratagene, La Jolla, Calif.). The coding sequence was cloned into pc3.1DNA(−) using EcoRV and PmeI restriction sites. The binding domain (BD) was created by PCR using genomic DNA as a template. Primers included unique restriction sites for directed cloning. The PCR product was cloned into an existing PTM plasmid (PTM-CF24, pc3.1DNA) using NheI and SacII restriction sites. This plasmid already contained the remaining elements of the TSD including a spacer sequence, polypyrimidine tract (PPT), branchpoint (BP) and 3′ acceptor site. The whole of the TSD was then subcloned into the vector (described above) containing the factor VIII PTM coding sequences. Finally, bovine growth hormone 3′ untranslated sequences from a separate plasmid clone were subcloned into the above PTM using PmeI and BamHI restriction sites.

The whole construct was sequenced and then analyzed by RT-PCR for possible cryptic splicing, and then subcloned into the AAV plasmid pDLZ20-M2 using XhoI and BamHI restriction sites (Chao et al., 2000, Gene Therapy 95:1594-1599; Flotte and Carter, 1998, Methods Enzymol., 292:717-32). For some viral (and non-viral) delivery systems, the size of the therapeutic is essential. Viral vectors such as adeno-associated virus are preferred because they are a (i) non-pathogenic virus with a broad host range (ii) it induces a low inflammatory response when compared to adenovirus vectors and (iii) it has the ability to infect both dividing and non-dividing cells. However, the packaging capacity of the rAAV is limited to approximately 110% of the size of the wild type genome, or ˜4.9 kB, thus, leaving little room for large regulatory elements such as promoters and enhancers. The B-domain deleted human factor VIII is close to the packaging size of AAV, thus, trans-splicing offers the possibility of delivering a smaller transgene while permitting the addition of regulatory elements.

To eliminate cryptic donor sites in the pre-mRNA upstream of the XhoI PTM cloning site approximately 170 bp of sequence was eliminated from the original AAV construct that includes part of exon 1 and all of the intron 1 sequence (see FIG. 44C).

The repair model in FIG. 44D shows a simplified model of the mouse factor VIII pre-mRNA target (endogenous gene) consisting of exons 1-14, intron 14, exon 15, intron 16, and exon 16-24 containing a neomycin gene insertion. The PTM shown in the figure consists of exon 16-24 coding sequences and a trans-splicing domain with its own splicing elements (donor site, branchpoint and pyrimidine tract) and a binding domain. Details of the binding domain are shown in FIGS. 44A and 44B. The binding domain is complementary to the splice site of intron 15 and part of exon 16 (5′ end).

The key advantages of using 3′ exon replacement for gene repair are (i) the construct requires less sequence and space than a full length gene construct, thereby leaving more space for regulatory elements, (ii) SMaRT repair should only occur in those cells that express the target gene, therefore eliminating any potential problems associated with ectopic expression of repaired RNA.

Factor VIII deficient mice were maintained at the animal facilities at the University of North Carolina at Chapel Hill. For plasmid injections each mouse was sedated and placed under a dissecting microscope and a 1 cm vertical midline abdomen incision was made. Approximately 100 micrograms of PTM plasmid DNA in phosphate buffered saline was injected to liver portal vein. Blood was collected from the retro-orbital plexus at intervals of 1, 2, 3 and 20 days after injection and assayed for factor VIII activity using the Coatest assay.

Factor VIII activity in blood samples collected from mice were assayed using a standard test called the Coatest assay. The assay was performed according to manufacturer's instructions (Chromgenix AB, Milan, Italy). Data indicating repair of factor VIII in factor VIII knock out mice is demonstrated in FIG. 46.

Hemophilia A defects in humans are broadly split into several categories that include gross DNA rearrangements, single DNA base substitutions, deletions and insertions. It has been determined that a rearrangement of DNA involving an inversion and translocation of exons 1-22 (together with introns) away from exons 23-26 is responsible for ˜40% of all cases of severe hemophilia A. The canine hemophilia A model also has a very similar gross rearrangement. This mutation will be used as the basis for our human and canine factor VIII PTM designs.

Methods for building the human factor VIII PTM will be very similar to that described above for the mouse PTM except that different coding regions (exons 23-26) will be amplified from a human cDNA, the binding domain will be amplified from human genomic sequence templates (whole genomic DNA or a genomic clone), and a C-terminal FLAG tag will be engineered in the PTM to be used to detect repaired factor VIII protein. The remaining elements of the trans-splicing domain including a spacer sequence, polypyrimidine tract (PPT), branchpoint (BP) and 3′ acceptor site will be obtained from an existing plasmid. Where necessary changes will be made to the binding domain sequence to eliminate any cryptic splicing within the PTM. The final PTM will be subcloned into the same mouse AAV plasmid vector, pDLZ20-M2 and virus preparation made from this plasmid. The canine factor VIII PTM will be made in an identical fashion but using canine cDNA and genomic plasmid.

### EXAMPLE

### Targeted Trans-Splicing of Papilloma Viral RNA

The vast majority of cervical cancers are associated with oncogenic human papilloma viruses (HPVs) and express viral mRNAs encoding the E6 and E7 oncoproteins. As described below, PTMs targeted against the E6 region of HPV-16 and splice the TM exon to the 5′ end of the E6 ORF using the 5′ splice site at the nucleotide 226.

Materials and Methods

The target DNA (p1059) was used to test PTM efficiency and contains the entire HPV-16 early region (nt 79-4468) cloned behind the SV40 early promoter and origin of replication. Specificity was assessed using the heterologous expression vector lacZCF9m as target (Puttaraju et al. 2001. Mol Ther 4:105-14). Plasmids were prepared using Quiagen maxi prep kits.

Nearly confluent 6 cm plates of 293 cells were transfected with 2 μg target DNA and 2 μg PTM DNA using LipofectAmine 2000 (Life Technologies). At two days post-transfection, cells were washed on the plate with PBS and lysed on the plate using 300 μl lysis buffer. Total cell RNA was prepared using Ambion RNAqueous kit. Transfected DNA was removed from the RNA by LiCl precipitation followed by DNAse I treatment using the Amboin DNA-free™ DNAse treatment and removal reagents.

RNA was converted to cDNA using RT from the High Capacity cDNA Archive Kit (PE Applied Biosystems) as directed by the manufacturer with the following modifications: the amount of random primer was cut in half and 5 μl of a 50 μM stock of oligo(dT16) and 5 μl of a 20 unites/:l stock of RNAse inhibitor were added per 100 μl reaction. RT reactions were diluted to 50 ng/μl and 5 ng/μl (based on original RNA content) for real time Quantitative PCR (QPCR) analysis. Amounts of specific cis and trans spliced mRNAs were quantitated using Real Time Quantitative PCR. These assays are referred to as Real Time QRT-PCR. These reactions were carried out on the Bio-Rad iCycler iQ Real Time PCR instrument using the SYBR Green kit from PE Applied Biosystems essentially as described previously (Puttaraju et al. 2001 Mol Ther 4:105-14.).

Total HPV-16 RNA levels (cis and trans-spliced) were assessed using a common amplicon in E6 exon 1 (HPV-16 nt 152-204; 53 bp). This assay uses the HPV-16 primers oJMD-15 (ACAGAGCTGCAAACAACTAT) and oJMD-16 (TTGCAGTACACATTCTAA). The amount of RT reaction used for each PCR reaction was 5 ng. Trans-splicing from the HPV-16 nt 226 5′ splice site to the PTM lacZ exon was assessed using a 53 bp chimeric amplicon. This assay uses the HPV-16 senser primer oCCB-348 (GCAAGCAACAGTTACTGCGA; HPV-16 nt 201-220) and the lacZ antisense primer oCCB-322 (ATCCACCCAGTCCCAGA). The amount of RT reaction used for each PCR reaction was 50 ng. Both assays used the same plasmid (p3671) to generate standard curves for quantitation. Trans-splicing from the HPV-16 nt 880 5′ splice site to the PTM lacZ exon was assessed using a 50 bp chimeric amplicon. This assay uses the HPV-16 sense primer oCCB-366 (ATCTACCATGGCTGATCCTG; HPV-16 nt 858-877) and the lacZ antisenser primer oCCB-322. The amount of RT reaction used for each PCR reaction was 50 ng. The plasmid p3672 was used to generate the standard curve for this assay.

Plasmids used as standards for real time QPCR were cloned as follows. An RT reaction from cotransfections of p1059 and HPV-PTM1 in 293T cells was used as template for PCR reactions. Primers oCCB-257 (HPV-16 nt 127-147; ACCCAGAAAGTTACCACAGTT) and oCCB-322 gave a 127 bp band which was TOPO-cloned into pCRII-TOPO (Invitrogen) to give p3671. Sequencing showed that this DNA corresponds to trans-splicing from HPV-16 nt 226 into the 3′ splice site of the PTM. Primers oJMD-17 (HPV-16 nt 689-708; GACAAGCAGAACCGGACAGA) and oCCB-322 gave a 219 bp band which was TOPO-cloned into pCRII-TOPO to give p3672. Sequencing showed that this DNA corresponds to trans-splicing from HPV-16 nt 880 into the 3′ splice site of the PTM. Plasmids stocks (1 ng/:l) were quantitated using PicoGreen (Molecular Probes) prior to use for standard curves.

Quantitation of cis- and trans-splicing for the cotransfections with PTMs and the target lacZCF9m were done exactly as described previously (Puttaraju et al. 2001. Mol. Ther. 4:105-14). The amount of RT reaction used for each PCR reaction was 5 ng.

Results

HPV and CF PTMs were contransfected into 293 cells with either the HPV-16 expression vector p1059 to assess trans-splicing efficiency or with lacZCF9m (containing a CF intron) to assess trans-splicing specificity. Real Time QRT-PCR assays were done as described above to assess levels of trans-splicing relative to cis-splicing of each target. The results are shown in Table 3. All RNA levels are expressed as fg of the DNA standard. The standards p3671 and p3672 are close to the same size so these values can be used to represent relative RNA levels for each assays. HPV-PTM1, 2, 5, and 6 efficiently trans-spliced to the HPV-16 nt 226 5′ splice site. Up to 70% trans-splicing was seen for the HPV-PTM1. As expected, HPV-PTM5 trans-splicing was abolished by mutations in the branch point and polypyrimidine tracts of the PTM. These PTMs showed less than 1% trans-splicing to the nt 880 5′ splice site. This data is consistent with the design of these PTMs which have binding domains complementary to the nucleotide 409 and 526 3′ splice sites. HPV-PTM-8 and HPV-PTM-9 trans binding domains downstream of the nt 880 5′ splice site and show efficient trans-splicing to this 5′ splice site (37% for HPV-PTM8 and 22% for HPV-PTM9) and somewhat less efficient trans-splicing to the nt 226 5′ splice site. HPV-PTM9 may interfere sterically with binding of splicing factors to the nt 880 5′ splice site. The specificity of HPV-PTM1, 2, 5 and 6 was also assessed by their ability to trans-splice to a target pre-mRNA with a CF intron. Specificity ranged from 274 to 606 fold.

### EXAMPLE

### Design of Targeted Papilloma virus PTMs

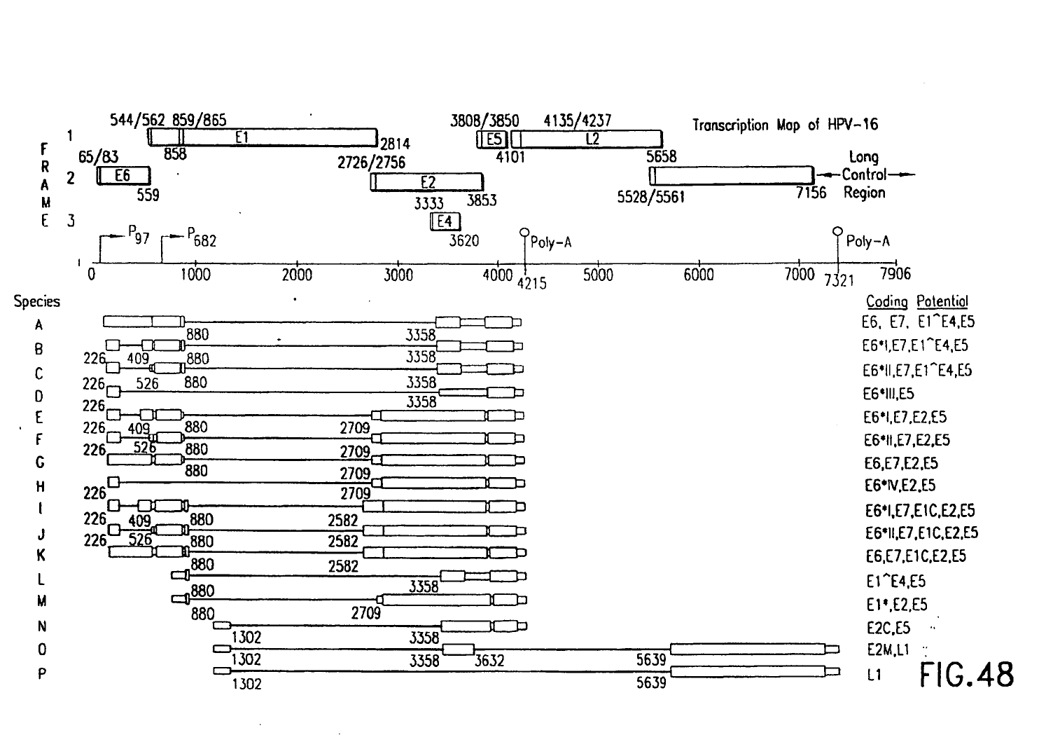

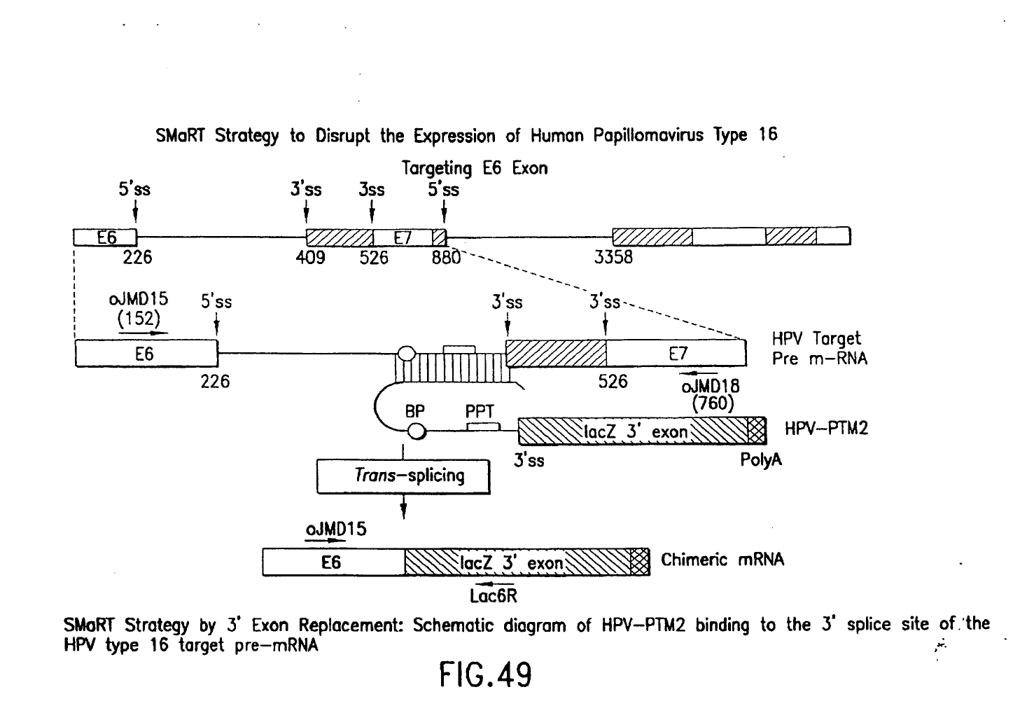

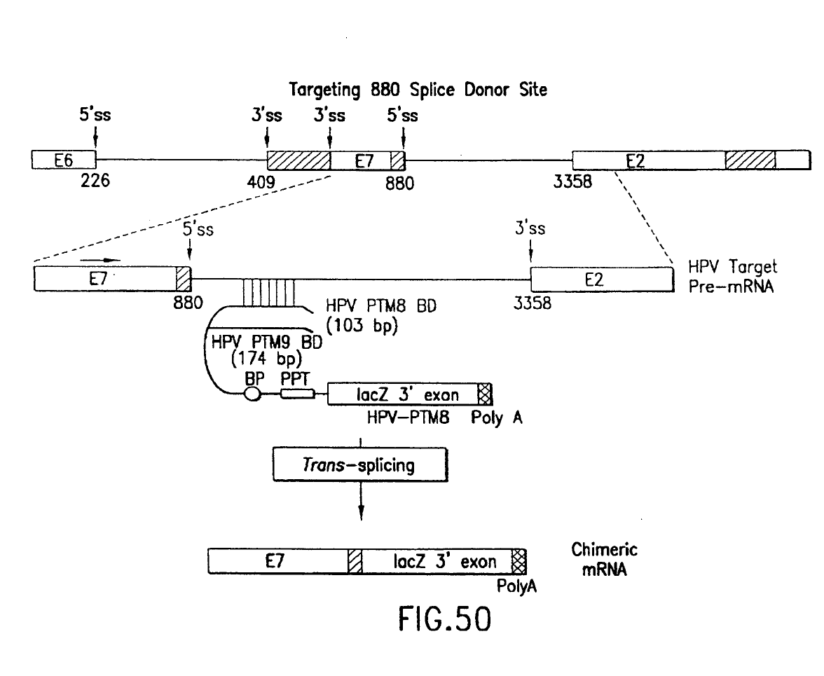

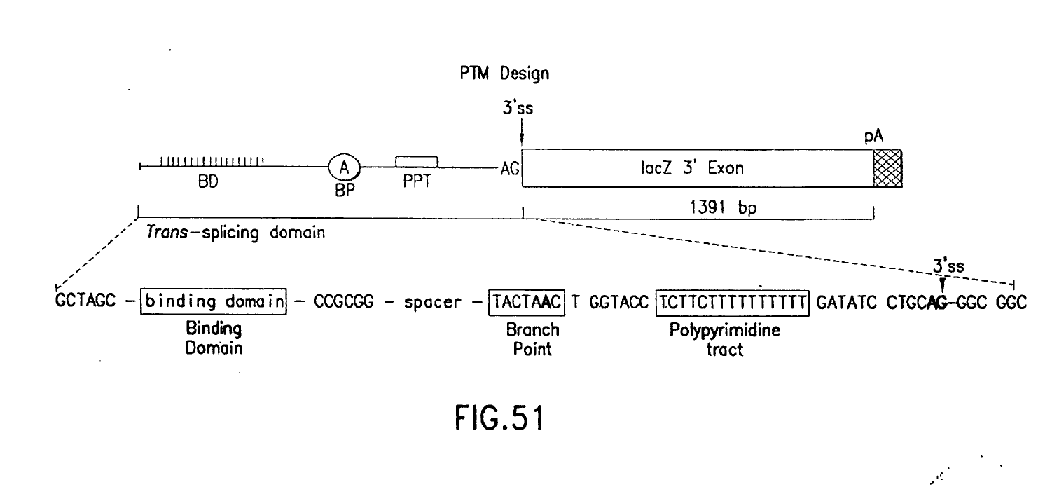

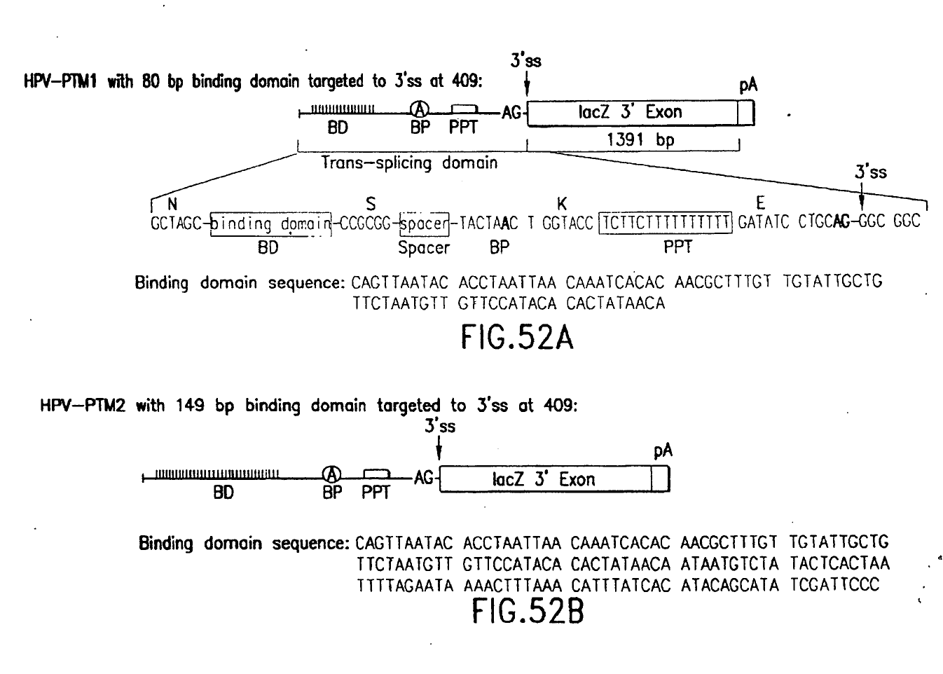

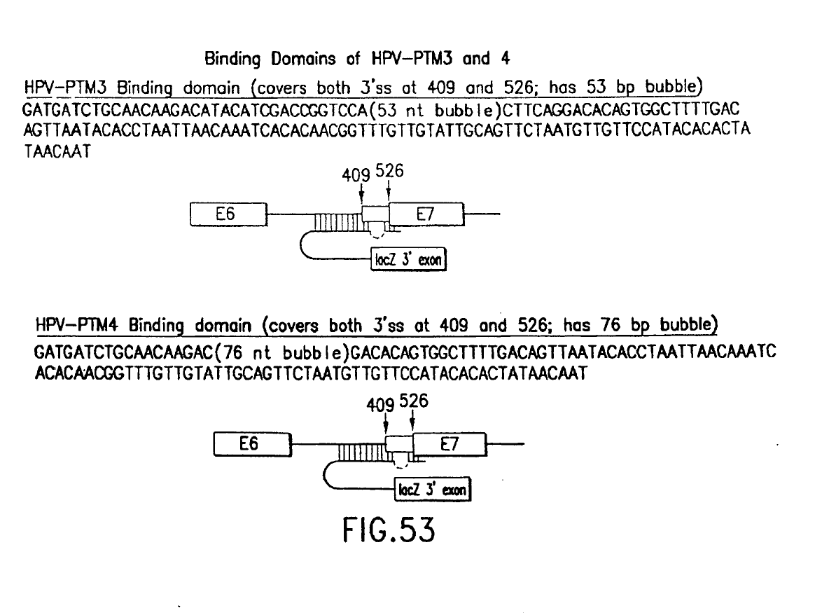

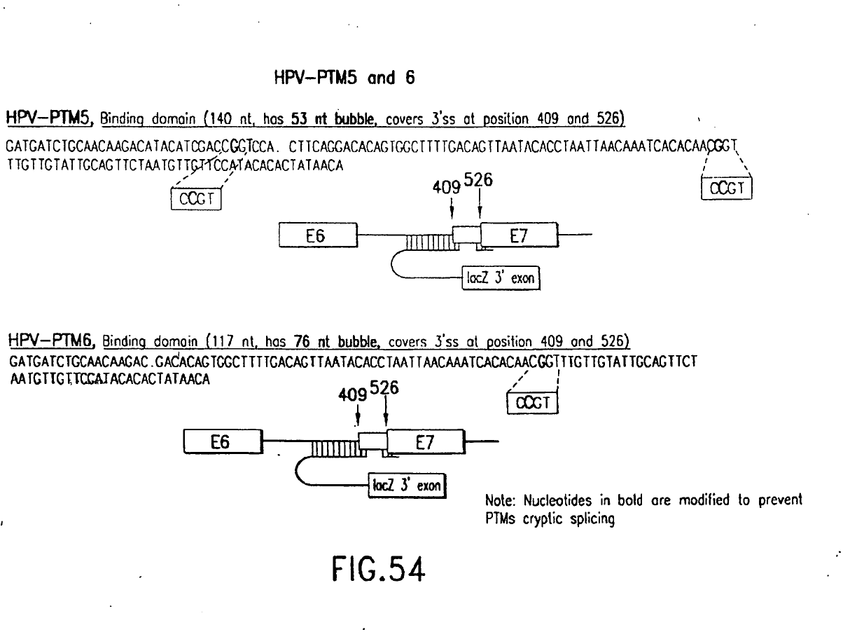

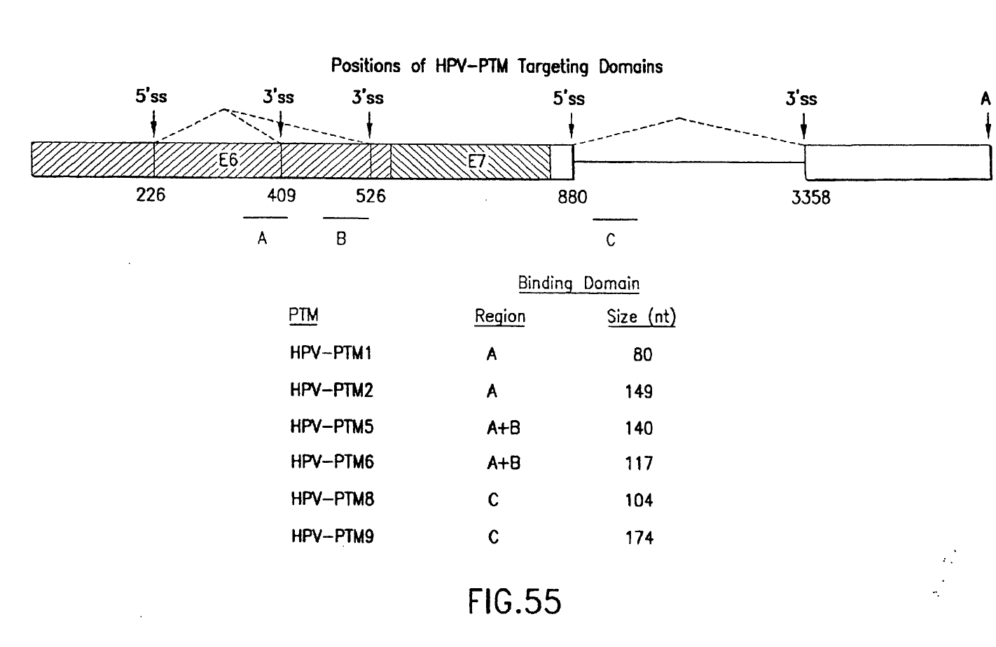

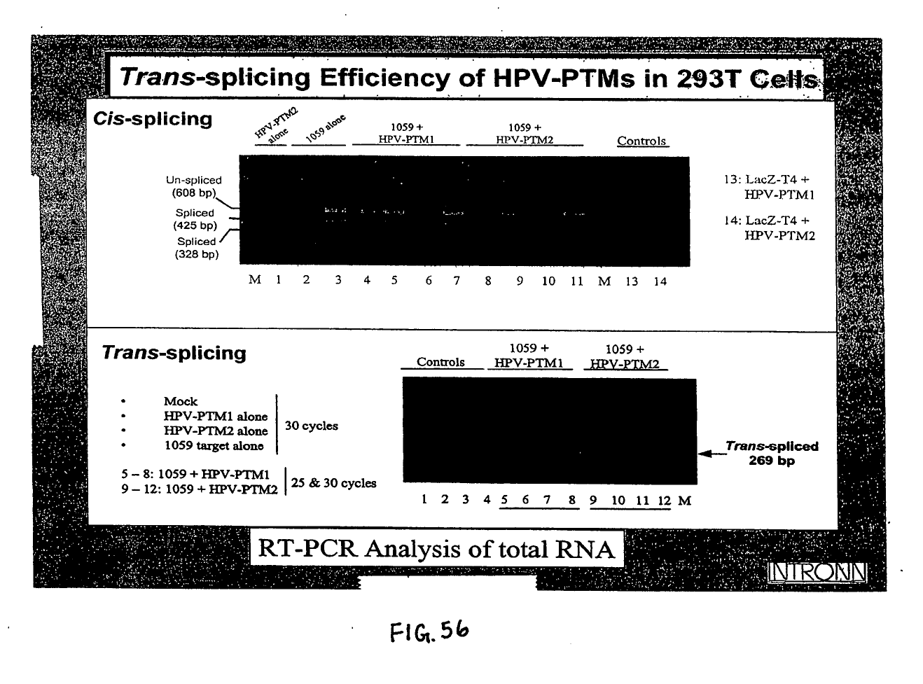

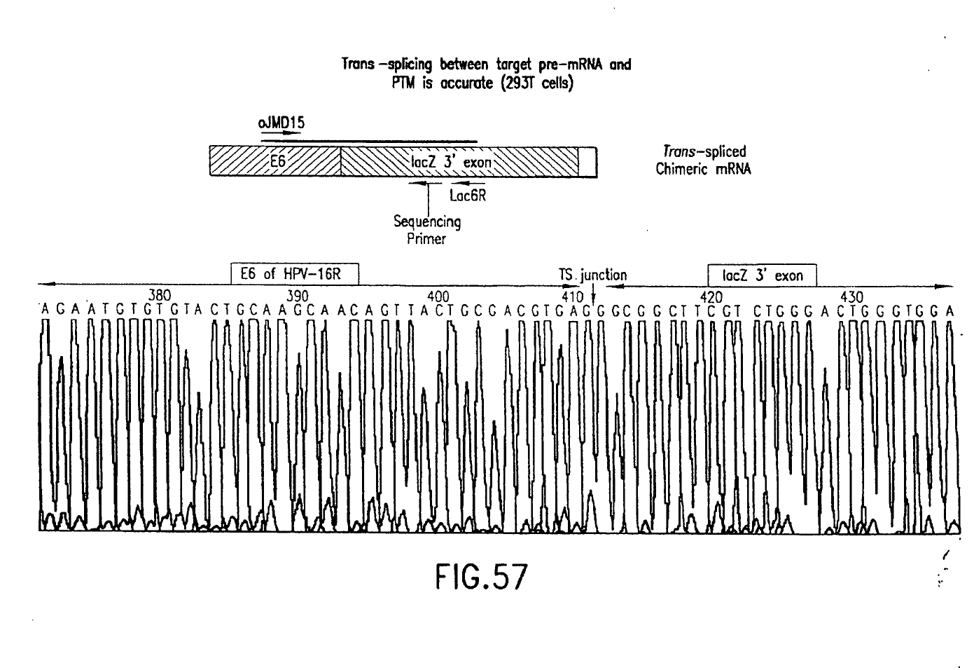

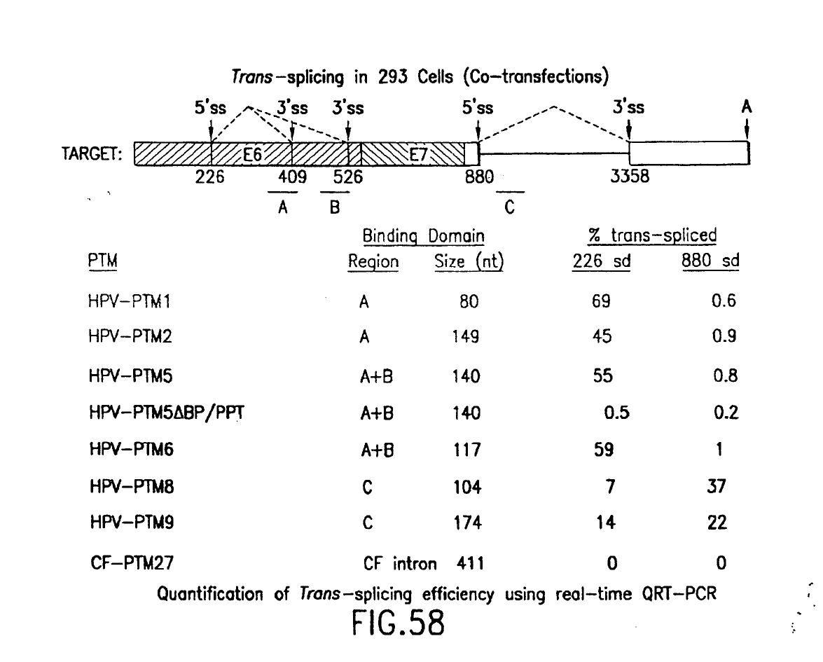

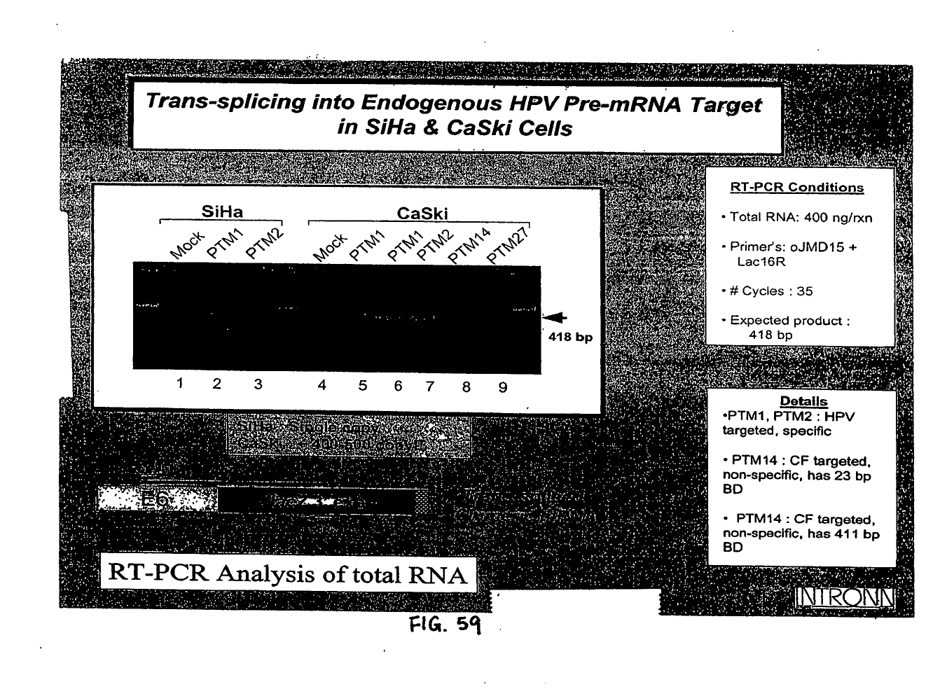

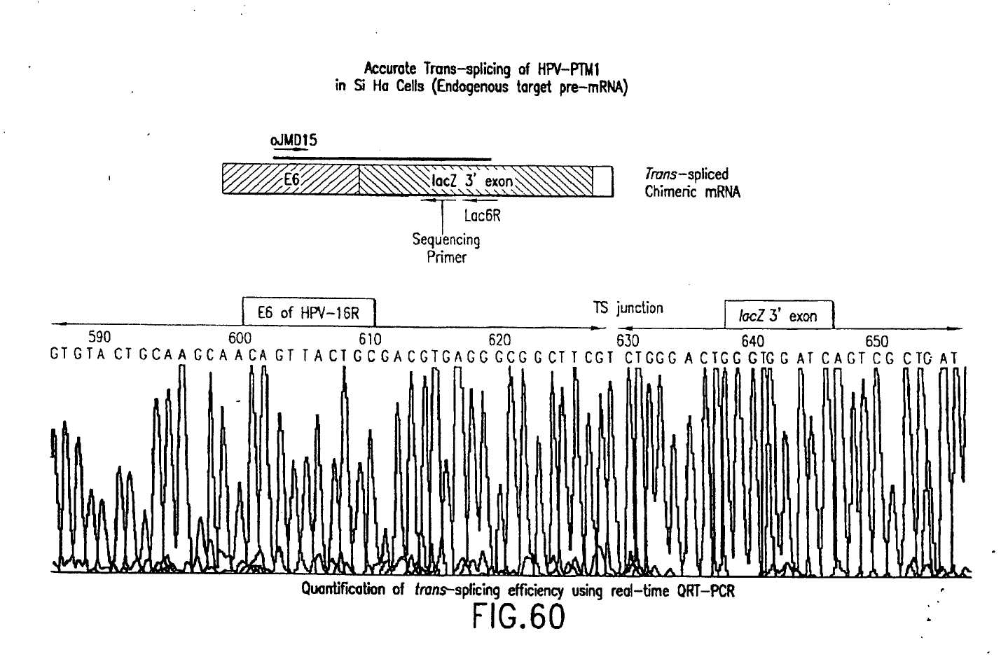

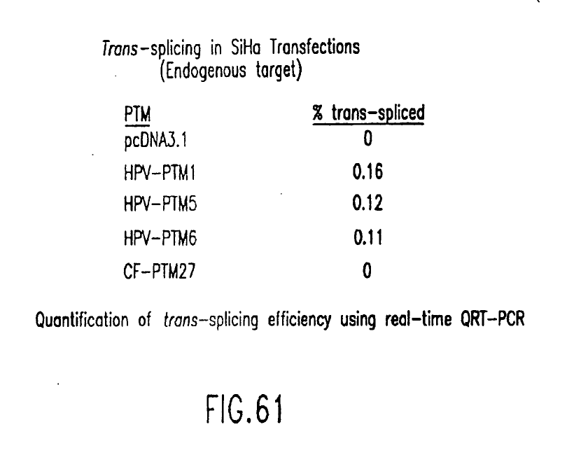

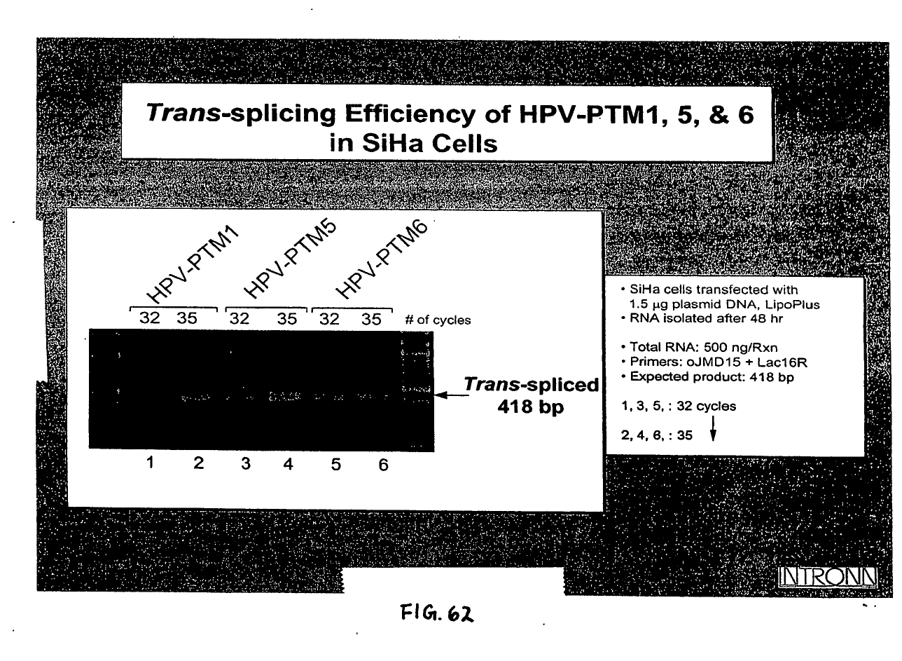

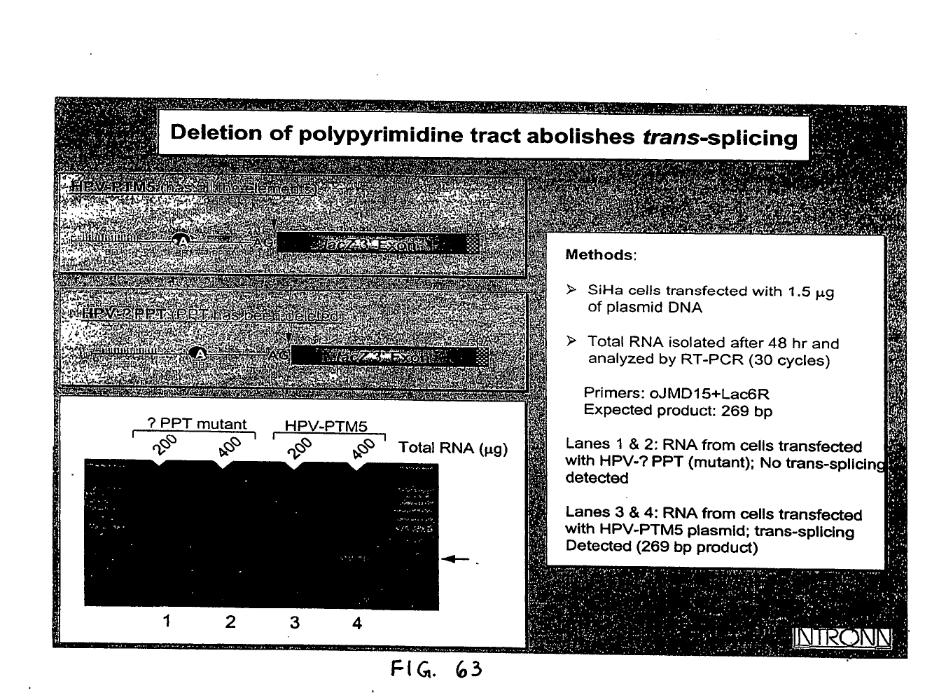

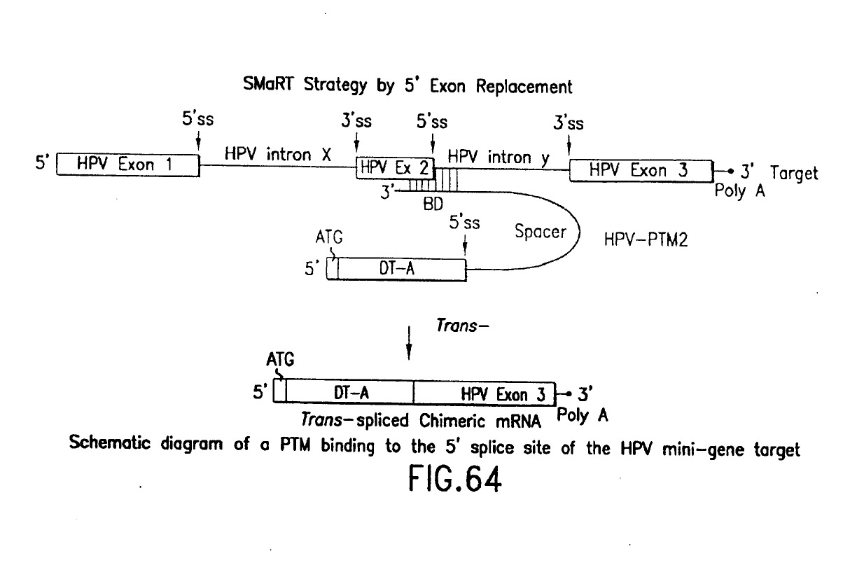

Initial pre-therapeutic RNA molecules (“PTMs”) are developed based on the abundance and splicing patterns of HPV mRNA. The transcription map of HPV-16 in benign infections is shown in FIG. 48. Cis and trans splicing assays are performed on the initial PTMs and the data obtained from the assays is used to create specific PTMs with optimized efficacy in spliceosome-mediated RNA trans-splicing reactions.

The most effective PTM is one that trans-splices an HPV target transcript with a PTM encoding a toxic product which will kill the infected cell. In targeting the most frequently used HPV splice sites, two viable 5′ splice site targets and two viable 3′ splice site targets can be used. Less frequently used splice sites can also make good targets if the PTM is designed to block the more frequently used site. Choice of target splice sites is further restricted if the intention is to treat cancers, since integration of HPV-16 in many cervical cancers leads to expression of only the E6 and E7 regions in these cancers.

The following target splice sites are used in the development of the initial PTMs which leads to the expression of a toxic product:

i) 5′ splice site targets:
nt 226: This splice site is used in the synthesis of all E6* species. In most tumors and cell lines, the vast majority of P97 promoter transcripts will be spliced using this 5′ splice site.
nt 880: This splice site is used in the synthesis of all E6US (unspliced) and E6* species except E6*III, both splice sites are good targets in both productive infections and cancers; and

ii) 3′ splice site targets:
nt 409: This 3′ splice site is used in the splicing of E6*I species which are generally more abundant than E6*II species. This splice site is used in cancers and productive HPV infection.
nt 3358: This target is used for splicing of most mRNAs, but only if the viral DNA is extrachromosomal. This splice site is not a good target for the treatment of most cancers.

In addition, a double trans-splicing PTM is developed to replace the internal exons nt 409-880 or nt 526-880 in productively infected tissue and in cancers.

Alternatively, initial PTMs are designed in which trans-splicing produces an mRNA encoding a fusion protein that is part viral and part exogenous peptide encoded by the PTM. The fusion protein will change the function of the viral protein so that it inhibits an essential viral function. The splice sites listed above are targeted to produce three viral fusion proteins:
(i) The E6 N terminus, using the nt 226 5′ splice site as the target;
(ii) The E6 C terminus, using the nt 409 (best) or nt 526 3′ splice sites as the targets; and
(iii) The E2 C terminus, using the nt 3358 3′ splice site as target.

This fusion protein is produced in productive infections and cancers containing extrachromosomal viral DNA. The C terminal domain of E2 is the DNA binding and dimerization domain, and can be used to target a fusion protein to the P97 promoter and block transcription. At high concentrations, the E2 viral protein binds just upstream of the P97 promoter and inhibits transcription by competing with the transcription factors, SP1 and TFIID, for binding. However, these E2 binding sites are weaker than those upstream in the Long Control Region (LCR) and are only saturated at high concentrations of the viral E2 protein. At low concentrations of E2, the protein binds to the E2 binding sites in the upstream LCR and activates transcription. Thus, a “repressor” domain can be added to the fusion protein resulting in a block of transcription through binding to any E2 binding site. This fusion protein is also useful to block viral DNA replication, since an E1/E2 complex binds the origin of replication. It has been demonstrated, however, that a complex of the E2 DNA binding domain and E1 does not bind to the origin. Since E2 is a dimer, heterodimerization of the E2 fusion protein with full length E2 protein would probably eliminate E2 function in DNA replication.

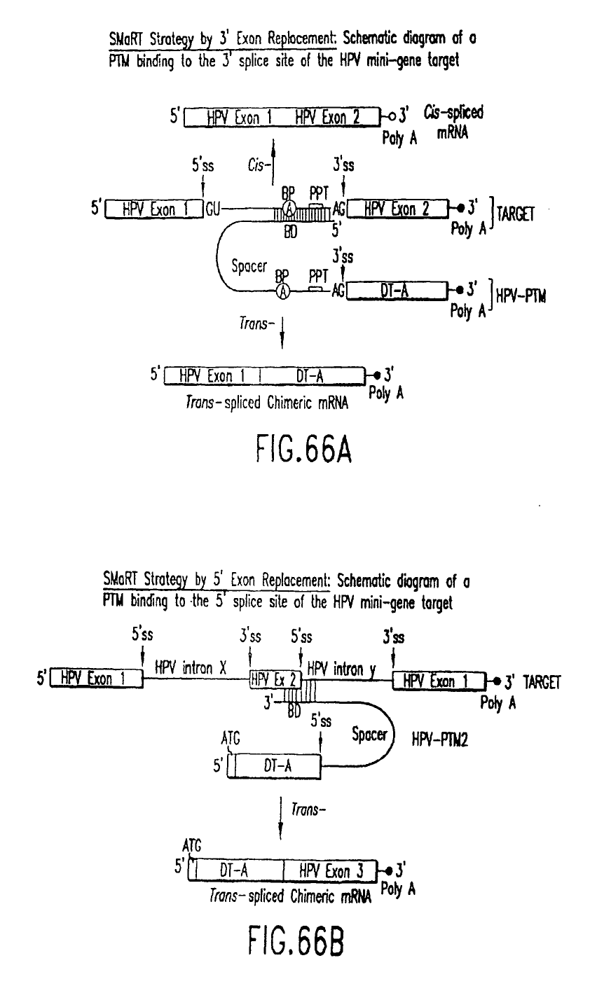

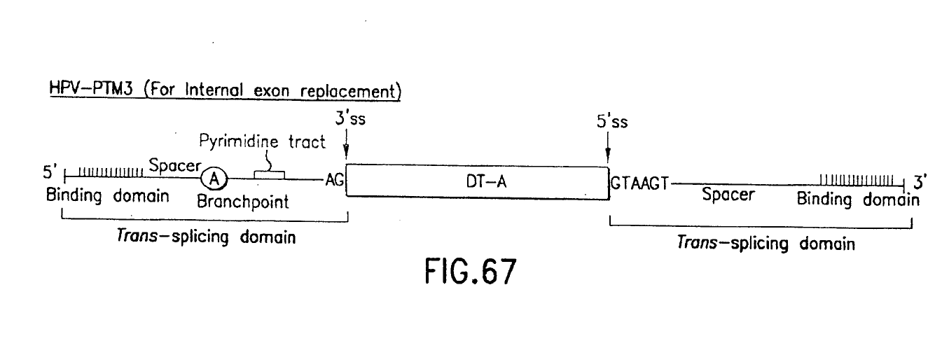

PTM(s) based on their ability to target and trans-splice to the HPV target splice sites depicted in FIG. 48 listed above are constructed and screened such that splicing results in the expression of diphtheria toxin sub unit A (DT-A) product, which will kill the infected cells or express a marker gene which can be easily detected. Other peptide or protein toxins may also be encoded. A typical prototype PTM (3′ trans-splicing) consists of an antisense target binding domain (25 or more) complementary to HPV sequences, spacer sequence, canonical branchpoint sequence (UACUAAC), an extensive polypyrimidine tract (12-15 U's), AG dinucleotide of the 3′ splice site followed by the delivered gene. PTMs are also constructed to carry out PTM-mediated trans-splicing with HPV 3′ splice sites (FIG. 66B). The trans-splicing domain (TSD) of the PTMs are constructed in modular fashion. Unique restriction sites are incorporated between each of the PTM elements, facilitating the replacement of individual elements. Schematic diagrams of 3′ exon replacement and 5′ exon replacement models are shown (FIG. 66A-B), respectively. It has previously been demonstrated that both efficiency and specificity of trans-splicing can be modulated substantially by altering several sequences in the TSD, including, the length of the binding domain, spacer sequences, strength of the PPT etc.

“Linear” PTMs are designed initially to maximize the trans-splicing efficiency, thereby identifying the PTM sequences that provide highest trans-splicing efficiency. Linear PTMs refer to the binding domain in the PTM as single stranded in configuration To achieve a higher degree of targeting specificity, another form of TSD referred to as a “safety stem” can be constructed. In these PTMs, the splice site of the PTM is protected from reacting with other pre-mRNA targets by binding to itself in a folded structure. Contact with the specific target promotes unwinding of the safety stem exposing and activating the PTM's 3′ splice site for spliceosome formation.

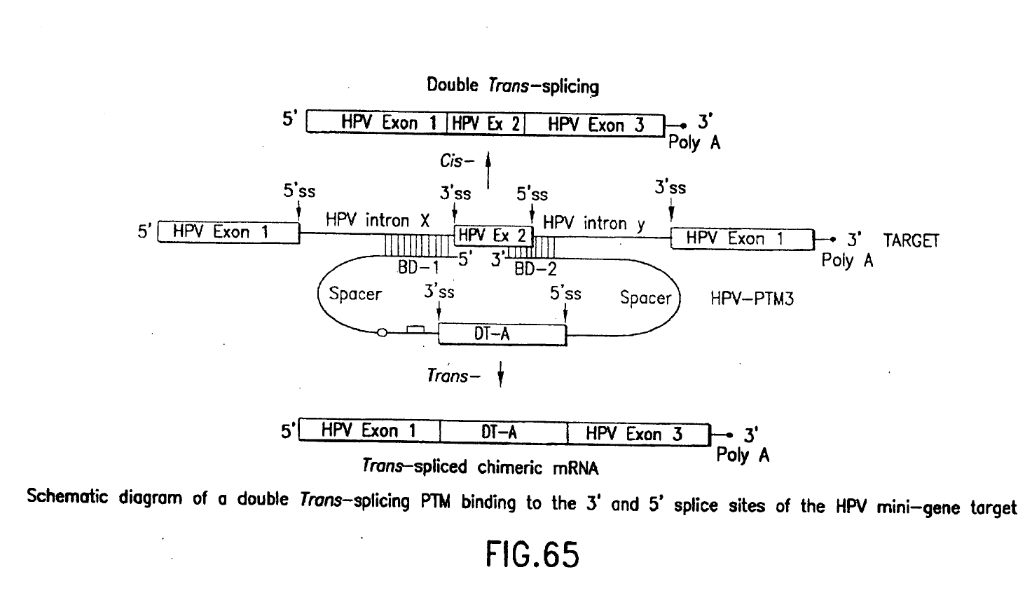

To further enhance the trans-splicing specificity, a PTM that requires two trans-splicing events to produce the expected therapeutic effect is also constructed (FIG. 65). This PTM will have an upstream 3′ splice site that will trans-splice into an HPV 5′ splice site, producing a singularly trans-spliced product. This product does not contain the required polyadenylation signals and would be inactive due to failure in nucleocytoplasmic transport and translation of the mRNA. A second trans-splicing event with a HPV 3′ splice site is necessary to provide the PTM with the signals required for polyadenylation (FIG. 65). Polyadenylation is necessary for PTMs with linear binding domains in which both 3′ and 5′ binding domains are linear, or with 3′ safety+5′ linear binding domains, are also designed for inhibition of viral expression. In addition, PTMs are designed as “double safety” PTMs with both 3′ and 5′ safety splice sites or 3′ linear or 5′ safety sites.

Testing of the PTMs is performed using in vitro splicing assays and cell culture-based assays. HPV-16-containing cell lines are used for testing the ability of PTMs to trans-splice. W12 cells (80263 cells) contain extrachromosomal HPV-16 DNA and express the full HPV-16 early region and can be used to test PTMs targeting. SiHa and CaSki cell lines contain integrated HPV-16 and express only the viral E6/E7/5′E1 regions. These cell lines are useful because they express viral pre-mRNAs characteristic of those expressed in cervical cancers. However, they may not be useful cell lines for testing a PTM targeting the nt 3358 3′ splice site. CaSki cells express considerably higher levels of HPV-16 mRNAs than any of the other cell lines tested and therefore may be the best cells for assaying other PTMs.

Cell culture-based cotransfection experiments with a PTM expression vector and an HPV-16 early region expression vector are assayed for expression of the PTM. Several plasmids driving the expression of HPV-16 have been constructed. For example, two plasmids that can be used in co-transfection experiments include ones that express HPV-16 under the direction of either the SV40 early promoter (p1059) or the K14 promoter (p2571; pK14-1203).

Combined isoform-specific (i.e. splice-specific) primers with quantitative real time reverse transcription polymerase chain reaction (QRT/PCR) are used to assay for alternative splicing. This assay is very isoform specific, relatively insensitive to RNA degradation, sensitive to one molecule of cDNA, has a wide dynamic range (at least seven orders of magnitude), and gives absolute quantitation of each isoform. Primer pairs specific for each PTM/target pre-mRNA combination are used. The sequence specificity of the assay permits the monitoring of the specificity of the trans-splicing reactions. The sensitivity and quantitative nature of the assay as well as the rapidity with which assays can be developed and performed is useful for the optimization of PTMs targeted against papillomaviral pre-mRNAs.

The specificity of PTM induced trans-splicing (i.e. to determine the specificity of targeted trans-splicing to HPV target pre-mRNA) is also evaluated by 5′ and/or 3′ rapid amplification of cDNA ends (RACE) according to standard procedures. This method is relatively fast compared to the conventional cDNA library construction, and gives complete sequences of 5′ and/or 3′ cDNA ends, so that the number of specific and non-specific splicing events can be determined. Initially, two cDNA libraries are constructed comprised of RNA isolated from cells co-transfected with a (i) linear PTM+HPV mini-gene target and (ii) safety PTM+HPV mini-gene target. For example, in order to identify the 5′ ends of the trans-spliced RNAs (3′ exon replacement), a 5′ RACE assay is performed with a PTM antisense primer. Similarly, to identify the 3′ ends of the trans-spliced RNAs (5′ exon replacement), a 3′ RACE assay is performed using a PTM sense primer. The cDNA is amplified by PCR, digested with restriction enzymes and cloned into a plasmid vector. The cDNA clones are initially screened by colony hybridization using a PTM specific probe. From each cDNA library, positive clones are selected and sequenced, and the sequence information is used to compare the specificity of linear vs. safety PTM. This permits identification of non-specific targets that trans-splice at high frequencies. Analysis of these targets provides useful information about the sequences that are responsible for non-specific trans-splicing and helps in the construction of specific PTMs.

The trans-splicing efficiency and specificity data obtained from the analysis of the initial candidate PTMs in trans-splicing assays is used to formulate and develop PTMs with optimal trans-splicing capabilities. The optimal PTMs are analyzed using the trans-splicing assays described above.

A mouse model for papillomavirus infections using an organotypic “graft/xenograft” technique. Papillomaviruses are generally species and cell type specific. Productive infections have been established in nude mice using bovine keratinocytes and bovine papillomaviruses. In this system, keratinocytes are initially plated onto a collagen “raft” containing fibroblasts and allowed to grow to confluence in tissue culture. The keratinocytes are then infected or transfected with papillomavirus or viral genomic DNA, respectively, and allowed to grow in culture for a few days. These rafts are then grafted onto the backs of nude mice where they develop into productively infected bovine tissue. Human papillomavirus infections can be established using the same techniques combined with human papillomaviruses and keratinocytes. This system is useful for testing the in vivo efficacy of anti-papillomavirus PTMs. In addition, grafting of cervical carcinoma tissue or cervical cancer cell lines onto nude mice is used. In addition, testing can be done using several animal models including bovine papillomavirus (BPV-1), Canine oral papillomavirus (COPV), and Cottontail rabbit papillomavirus (CRPV). COPV, in particular, has served as a good model for vaccine development.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

We claim:

1. A cell comprising a nucleic acid molecule wherein said nucleic acid molecule comprises:
a) one or more target binding domains that target binding of the nucleic acid molecule to a human papilloma virus pre-mRNA expressed within the cell;
b) a splice region;
c) a spacer region that separates the splice region from the target binding domain; and
d) a nucleotide sequence to be trans-spliced to the target pre-mRNA;
wherein said nucleic acid molecule is recognized by nuclear splicing components within the cell.

2. The cell of claim 1 wherein the splice region is a 3′ splice region comprising a branch point, a pyrimidine tract and a 3′splice acceptor site.

3. The cell of claim 1 wherein said nucleic acid molecule further comprises a safety nucleotide sequence comprising one or more complementary sequences that bind to one or both sides of the splice region.

4. The cell of claim 1 wherein the binding of the nucleic acid molecule to the target pre-mRNA is mediated by complementary, triple helix formation, or protein-nucleic acid interaction.

5. The cell of claim 1 wherein the nucleotide sequences to be trans-spliced to the target pre mRNA encode a human papilloma virus polypeptide.

6. The cell of claim 1 wherein the human papilloma virus is an oncogenic papilloma virus.

7. The cell of claim 1 wherein the human papilloma virus is papilloma virus 16.

8. A cell comprising a recombinant vector wherein said vector expresses a nucleic acid molecule comprising:
a) one or more target binding domains that target binding of the nucleic acid molecule to a human papilloma virus pre-mRNA expressed within the cell;
b) a splice region;
c) a spacer region that separates the splice region from the target binding domain; and
d) a nucleotide sequence to be trans-spliced to the target pre-mRNA;
wherein said nucleic acid molecule is recognized by nuclear splicing components within the cell.

9. The cell of claim 8 wherein the splice region is a 3′ splice region comprising a branch point, a pyrimidine tract and a 3′splice acceptor site.

10. The cell of claim 8 wherein said nucleic acid molecule further comprises a safety nucleotide sequence comprising one or more complementary sequences that bind to one or both sides of the splice region.

11. The cell of claim 8 wherein the binding of the nucleic acid molecule to the target pre-mRNA is mediated by complementary, triple helix formation, or protein-nucleic acid interaction.

12. The cell of claim 8 wherein the nucleotide sequences to be trans-spliced to the target pre mRNA encode a human papilloma virus polypeptide.

13. The cell of claim 8 wherein the human papilloma virus is an oncogenic papilloma virus.

14. The cell of claim 8 wherein the human papilloma virus is papilloma virus 16.

15. A method of producing a chimeric RNA molecule in a cell comprising:
contacting a target pre-mRNA expressed in the cell with a nucleic acid molecule recognized by nuclear splicing components wherein said nucleic acid molecule comprises:
a) one or more target binding domains that target binding of the nucleic acid molecule to a human papilloma virus pre-mRNA expressed within the cell;
b) a splice region;
c) a spacer region that separates the splice region from the target binding domain; and
d) a nucleotide sequence to be trans-spliced to the target pre-mRNA;
under conditions in which a portion of the nucleic acid molecule is trans-spliced to a portion of the target pre-mRNA to form a chimeric RNA within the cell.

16. The method of claim 15 wherein the splice region is a 3′ splice region comprising a branch point, a pyrimidine tract and a 3′splice acceptor site.

17. The method of claim 15 wherein said nucleic acid molecule further comprises a safety nucleotide sequence comprising one or more complementary sequences that bind to one or both sides of the splice region.

18. The method of claim 15 wherein the binding of the nucleic acid molecule to the target pre-mRNA is mediated by complementary, triple helix formation, or protein-nucleic acid interaction.

19. The method of claim 15 wherein the nucleotide sequences to be trans-spliced to the target pre mRNA encode a human papilloma virus polypeptide.

20. The method of claim 15 wherein the human papilloma virus is an oncogenic papilloma virus.

21. The method of claim 15 wherein the human papilloma virus is papilloma virus 16.

22. A nucleic acid molecule comprising:
a) one or more target binding domains that target binding of the nucleic acid molecule to a human papilloma virus pre-mRNA expressed within the cell;
b) a splice region;
c) a spacer region that separates the splice region from the target binding domain;
d) a safety sequence comprising one or more complementary sequences that bind to one or both sides of the splice site; and
e) a nucleotide sequence to be trans-spliced to the target pre-mRNA;
wherein said nucleic acid molecule is recognized by nuclear splicing components within the cell.

23. The nucleic acid molecule of claim 22 wherein the splice region is a 3′ splice region comprising a branch point, a pyrimidine tract and a 3′splice acceptor site.

24. The nucleic acid molecule of claim 22 wherein said nucleic acid molecule further comprises a safety nucleotide sequence comprising one or more complementary sequences that bind to one or both sides of the splice region.

25. The nucleic acid molecule of claim 22 wherein the binding of the nucleic acid molecule to the target pre-mRNA is mediated by complementary, triple helix formation, or protein-nucleic acid interaction.

26. The nucleic acid molecule of claim 22 wherein the nucleotide sequences to be trans-spliced to the target pre mRNA encode a human papilloma virus polypeptide.

27. The nucleic acid molecule of claim 22 wherein the human papilloma virus is an oncogenic papilloma virus.

28. The nucleic acid molecule of claim 22 wherein the human papilloma virus is papilloma virus 16.

29. A eukaryotic expression vector wherein said vector expresses a nucleic acid molecule comprising:
a) one or more target binding domains that target binding of the nucleic acid molecule to a human papilloma virus protein pre-mRNA expressed within the cell;
b) a splice region;
c) a spacer region that separates the splice region from the target binding domain; and
d) a nucleotide sequence to be trans-spliced to the target pre-mRNA; wherein said nucleic acid molecule is recognized by nuclear splicing components within the cell.

30. The vector of claim 29 wherein the nucleic acid molecule further comprises a 5′ donor site.

31. The vector of claim 29 wherein said vector is a viral vector

32. The vector of claim 29 wherein in said viral vector is an adeno-associated viral vector.

33. A method for inhibiting the expression of human papilloma virus pre-mRNA in a subject having cervical carcinoma comprising administering to said subject a nucleic acid molecule comprising:
a) one or more target binding domains that target binding of the nucleic acid molecule to a human papilloma virus pre-mRNA expressed within the cell; and
b) a nucleotide sequence to be trans-spliced to the target pre-mRNA;
wherein said nucleic acid molecule is recognized by nuclear splicing components within the cell.

34. A cell comprising a recombinant vector wherein said vector expresses a nucleic acid molecule comprising:
a) one or more target binding domains that target binding of the nucleic acid molecule to a human papilloma virus pre-mRNA expressed within the cell;
b) a splice site; and
c) a nucleotide sequence to be trans-spliced to the target pre-mRNA;
wherein said nucleic acid molecule is recognized by nuclear splicing components within the cell.

35. A method of producing a chimeric RNA molecule in a cell comprising:
contacting a target pre-mRNA expressed in the cell with a nucleic acid molecule recognized by nuclear splicing components wherein said nucleic acid molecule comprises:
a) one or more target binding domains that target binding of the nucleic acid molecule to a human papilloma virus pre-mRNA expressed within the cell;
b) a splice acceptor site; and
c) a nucleotide sequence to be trans-spliced to the target pre-mRNA;
under conditions in which a portion of the nucleic acid molecule is trans-spliced to a portion of the target pre-mRNA to form a chimeric RNA within the cell.


SEQUENCE LISTING (128 sequences)

SEQ ID NO: 1 - DNA, 132 bp, Homo sapien
caggggacgcaccaaggatggagatgttccagggcgctgatgatgttgttgattcttcttaaatcttttgtgatggaaaacttttcttcgtaccacgggactaaacctggttatgtagattccattcaaaaa

SEQ ID NO: 2 - DNA, 29 bp, Corynebacterium diptheriae
ggcgctgcagggcgctgatgatgttgttg

SEQ ID NO: 3 - DNA, 36 bp, Corynebacterium diptheriae
ggcgaagcttggatccgacacgatttcctgcacagg

SEQ ID NO: 4 - DNA, 68 bp, Artificial Sequence
  Oligonucleotide
aattctctagatgcttcacccgggcctgactcgagtactaactggtacctcttcttttttttcctgca

SEQ ID NO: 5 - DNA, 60 bp, Artificial Sequence
  Oligonucleotide
ggaaaaaaaagaagaggtaccagttagtactcgagtcaggcccgggtgaagcatctagag

SEQ ID NO: 6 - DNA, 24 bp, Artificial Sequence
  Oligonucleotide primer
tcgagcaacgttataataatgttc

SEQ ID NO: 7 - DNA, 24 bp, Artificial Sequence
  Oligonucleotide primer
tcgagaacattattataacgttgc

SEQ ID NO: 8 - DNA, 35 bp, Artificial Sequence
  Oligonucleotide primer
aattctctagatcaggcccgggtgaagcactcgag

SEQ ID NO: 9 - DNA, 25 bp, Artificial Sequence
  Oligonucleotide primer
tgcttcacccgggcctgatctagag

SEQ ID NO: 10 - DNA, 18 bp, Homo sapien
tgcttcacccgggcctga

SEQ ID NO: 11 - DNA, 16 bp, Homo sapien
ctcttcttttttttcc

SEQ ID NO: 12 - DNA, 18 bp, Homo sapien
caacgttataataatgtt

SEQ ID NO: 13 - DNA, 16 bp, Homo sapien
ctgtgattaatagcgg

SEQ ID NO: 14 - DNA, 16 bp, Homo sapien
cctggacgcggaagtt

SEQ ID NO: 15 - DNA, 51 bp, Homo sapien
ctgggacaaggacactgcttcacccggttagtagaccacagccctgaagcc

SEQ ID NO: 16 - DNA, 17 bp, Homo sapien
cttctgttttttttctc

SEQ ID NO: 17 - DNA, 16 bp, Homo sapien
cttctgtattattctc

SEQ ID NO: 18 - DNA, 16 bp, Homo sapien
gttctgtccttgtctc

SEQ ID NO: 19 - DNA, 29 bp, Corynebacterium diptheriae
ggcgctgcagggcgctgatgatgttgttg

SEQ ID NO: 20 - DNA, 36 bp, Corynebacterium diptheriae; identical to SEQ ID NO: 3

SEQ ID NO: 21 - DNA, 21 bp, Corynebacterium diptheriae
catcgtcataatttccttgtg

SEQ ID NO: 22 - DNA, 20 bp, Corynebacterium diptheriae
atggaatctacataaccagg

SEQ ID NO: 23 - DNA, 20 bp, Corynebacterium diptheriae
gaaggctgagcactacacgc

SEQ ID NO: 24 - DNA, 20 bp, Corynebacterium diptheriae
cggcaccgtggccgaagtgg

SEQ ID NO: 25 - DNA, 30 bp, Homo sapien
accggaattcatgaagccaggtacaccagg

SEQ ID NO: 26 - DNA, 20 bp, Homo sapien
gggcaaggtgaacgtggatg

SEQ ID NO: 27 - DNA, 19 bp, Homo sapien
atcaggagtggacagatcc

SEQ ID NO: 28 - DNA, 39 bp, Artificial Sequence
  Oligonucleotide primer complimentary to the Escherichia coli lacZ gene
gcatgaattcggtaccatgggggggttctcatcatcatc

SEQ ID NO: 29 - DNA, 36 bp, Artificial Sequence
  Oligonucleotide primer complimentary to the Escherichia coli lacZ gene
ctgaggatcctcttacctgtaaacgcccatactgac

SEQ ID NO: 30 - DNA, 38 bp, Artificial Sequence
  Oligonucleotide primer complimentary to the Escherichia coli lacZ gene
gcatggtaaccctgcagggcggcttcgtctgggactgg

SEQ ID NO: 31 - DNA, 38 bp, Artificial Sequence
  Oligonucleotide primer complimentary to the Escherichia coli lacZ gene
ctgaaagcttgttaacttattatttttgacaccagacc

SEQ ID NO: 32 - DNA, 47 bp, Artificial Sequence
  Oligonucleotide primer complimentary to the Escherichia coli lacZ gene
gcatggtaaccctgcagggcggcttcgtctaataatgggactgggtg

SEQ ID NO: 33 - DNA, 37 bp, Artificial Sequence
  Oligonucleotide primer complimentary to the beta HCG6 gene (accession #X00266)
gcatggatcctccggagggcccctgggcaccttccac

SEQ ID NO: 34 - DNA, 38 bp, Artificial Sequence
  Oligonucleotide primer complimentary to the beta HCG6 gene (accession #X00266)
ctgactgcagggtaaccggacaaggacactgcttcacc

SEQ ID NO: 35 - DNA, 35 bp, Artificial Sequence
  Oligonucleotide primer complimentary to the beta HCG6 gene (accession #X00266)
gcatggtaaccctgcaggggctgctgctgttgctg

SEQ ID NO: 36 - DNA, 37 bp, Artificial Sequence
  Oligonucleotide primer complimentary to the beta HCG6 gene (accession #X00266)
ctgaaagcttgttaaccagctcaccatggtggggcag

SEQ ID NO: 37 - DNA, 22 bp, Artificial Sequence
  Oligonucleotide primer complimentary to the Escherichia coli lacZ gene
ggctttcgctacctggagagac

SEQ ID NO: 38 - DNA, 21 bp, Artificial Sequence
  Oligonucleotide primer complimentary to the Escherichia coli lacZ gene
gctggatgcggcgtgcggtcg

SEQ ID NO: 39 - DNA, 20 bp, Artificial Sequence
  Oligonucleotide primer complimentary to the Escherichia coli lacZ gene
cggcaccgtggccgaagtgg

SEQ ID NO: 40 - DNA, 45 bp, Homo sapien
acctgggcccacccattattaggtcattatccgcggaacattata

SEQ ID NO: 41 - DNA, 35 bp, Homo sapien
acctctgcaggtgaccctgcaggaaaaaaaagaag

SEQ ID NO: 42 - DNA, 30 bp, Homo sapien
acctctgcagacttcacttctaatgatgat

SEQ ID NO: 43 - DNA, 51 bp, Homo sapien
acctgcggccgcctaatgatgatgatgatgatgctcttctagttggcatgc

SEQ ID NO: 44 - DNA, 32 bp, Homo sapien
gacctctcgagggatttggggaattatttgag

SEQ ID NO: 45 - DNA, 35 bp, Homo sapien
ctgacctgcggccgctacagtgttgaatgtggtgc

SEQ ID NO: 46 - DNA, 35 bp, Homo sapien
ctgacctgcggccgcccaactatctgaatcatgtg

SEQ ID NO: 47 - DNA, 32 bp, Homo sapien
gacctcttaagtagactaaccgattgaatatg

SEQ ID NO: 48 - DNA, 21 bp, Homo sapien
ctaatgatgatgatgatgatg

SEQ ID NO: 49 - DNA, 21 bp, Homo sapien
cgcctaatgatgatgatgatg

SEQ ID NO: 50 - DNA, 21 bp, Homo sapien
cttcttggtactcctgtcctg

SEQ ID NO: 51 - DNA, 32 bp, Homo sapien; identical to SEQ ID NO: 44

SEQ ID NO: 52 - DNA, 21 bp, Homo sapien
aactagaaggcacagtcgagg

SEQ ID NO: 53 - DNA, 24 bp, Artificial Sequence
  Trans-spliced product containing human chorionic gonadotropin gene 6 sequences and Corynebacterium diptheriae toxin A sequence
gagatgttccagggcgtgatgatg

SEQ ID NO: 54 - RNA, 127 nt, Artificial Sequence
  PTM intramolecular base paired stem
gcuagccugggacaaggacacugcuucacccgguuaguagaccacagcccugagccnnnnnnnnnnnnnnaucguuaacuaauaaacuacuaacugggugaacuucuguuuuuuucucgagcugcag

SEQ ID NO: 55 - RNA, 127 nt, Artificial Sequence
  PTM intramolecular base paired stem
gcuagccugggacaaggacacugcuucacccgguuaguagaccacagcccugagccnnnnnnnnnnnnnnaucguuaacuaauaaacuacuaacugggugaacuucuguauuauucucgagcugcag

SEQ ID NO: 56 - RNA, 127 nt, Artificial Sequence
  PTM intramolecular base paired stem
gcuagccugggacaaggacacugcuucacccgguuaguagaccacagcccugagccnnnnnnnnnnnnnnaucguuaacuaauaaacuacuaacugggugaaguucuguccuugucucgagcugcag

SEQ ID NO: 57 - DNA, 132 bp, Artificial Sequence; identical to SEQ ID NO: 1
  Trans-spliced product containing human chorionic gonadotropin gene 6 sequences and Corynebacterium diptheriae toxin A sequences

SEQ ID NO: 58 - DNA, 18 bp, Artificial Sequence
  Artificial sequence derived from Escherichia coli lacZ gene
gaattcggtaccatgggg

SEQ ID NO: 59 - DNA, 33 bp, Artificial Sequence
  Artificial sequence derived from Escherichia coli lacZ gene
cgtttacaggtaagaggatcctccggagggccc

SEQ ID NO: 60 - DNA, 30 bp, Artificial Sequence
  Artificial sequence derived from Escherichia coli lacZ gene
tggtgtcaaaaataataagttaacaagctt

SEQ ID NO: 61 - DNA, 25 bp, Artificial Sequence
  Trans-spliced product containing Esherichia coli lacZ and human chorionic gonadotropin gene 6 sequences
cagcagcccctgtaaacggggatac

SEQ ID NO: 62 - DNA, 286 bp, Artificial Sequence
  Trans-spliced product containing Escherichia coli lacZ gene sequences
ggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctaataatgggactgggtggatcagtcgctgattaaatatgatgaaaacgggcaacccgtggtcggcttacggcggtgattttggcgatacgccgaacgatcgccagttctgtatgaacggtctggtctttgccgaccgcacgccgcatccag

SEQ ID NO: 63 - DNA, 196 bp, Artificial Sequence
  Trans-spliced product containing Esherichia coli lacZ gene sequences
ggctttcgctacctggagagacgcgcccgctgatcctttgcgaatacgcccacgcgatgggtaacagtcttggcggtttcgctaaatactggcaggcgtttcgtcagtatccccgtttacaggggctgctgctgttgctgctgctgagcatgggcgggacatgggcatccaaggagccacttcggccacggtgccg

SEQ ID NO: 64 - DNA, 500 bp, Artificial Sequence
  Trans-spliced product comprising cystic fibrosis transmembrane regulator-derived sequences and His tag sequences
gctagcgtttaaacgggcccacccatcattattaggtcattatccgcggaacattattataacgttgctcgagtactaactggtacctcttcttttttttcctgcagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagagcatcatcatcatcatcattaggcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccac

SEQ ID NO: 65 - DNA, 20 bp, Artificial Sequence
  Splice junction sequence
atgttccagggcgtgatgat

SEQ ID NO: 66 - protein, 6 aa, Artificial Sequence
  Artificial sequence comprising sequences derived from Escherichia coli lacZ gene
DYKDDK

SEQ ID NO: 67 - DNA, 15 bp, Artificial Sequence
  Artificial sequence comprising sequence derived from Escherichia coli lacZ gene
ggagttgatcccgtc

SEQ ID NO: 68 - DNA, 37 bp, Artificial Sequence
  Artificial sequence comprising sequences derived from Escherichia coli lacZ gene
gcagtgtccttgtgcggttaccctgcagggcggcttc

SEQ ID NO: 69 - DNA, 120 bp, Artificial Sequence
  PTM binding domain of PTM
gattcacttgctccaattatcatcctaagcagaagtgtatattcttatttgtaaagattctattaactcatttgattcaaaatatttaaaatacttcctgtttcatactctgctatgcac

SEQ ID NO: 70 - DNA, 24 bp, Artificial Sequence
  Spacer sequence of PTM
aacattattataacgttgctcgaa

SEQ ID NO: 71 - DNA, 47 bp, Artificial Sequence
  Branch point, pyrimidine tract and acceptor splice site of PTM
tactaactggtacctcttcttttttttttgatatcctgcagggcggc

SEQ ID NO: 72 - DNA, 70 bp, Artificial Sequence
  Donor site and spacer sequence of PTM
tgaacggtaagtgttatcaccgatatgtgtctaacctgattcgggccttcgatacgctaagatccaccgg

SEQ ID NO: 73 - DNA, 260 bp, Artificial Sequence
  Binding domain of spacer sequence
tcaaaaagttttcacataatttcttacctcttcttgaattcatgctttgatgacgcttctgtatctatattcatcattggaaacaccaatgatttttctttaatggtgcctggcataatcctggaaaactgataacacaatgaaattcttccactgtgcttaaaaaaaccctcttgaattctccatttctcccataatcatcattacaactgaactctggaaataaaacccatcattattaactcattatcaaatcacgc

SEQ ID NO: 74 - DNA, 22 bp, Artificial Sequence
  Oligonucleotide primer
cgctggaaaaacgagcttgttg

SEQ ID NO: 75 - DNA, 23 bp, Artificial Sequence
  Oligonucleotide
actcagtgtgattccaccttctc

SEQ ID NO: 76 - DNA, 36 bp, Artificial Sequence
  Oligonucleotide
gacctctgcagacttcacttctaatgatgattatgg

SEQ ID NO: 77 - DNA, 33 bp, Artificial Sequence
  Oligonucleotide primer
ctaggatcccgttcttttgttcttcactattaa

SEQ ID NO: 78 - DNA, 33 bp, Artificial Sequence
  Oligonucleotide primer
ctagggttaccgaagtaaaaccatacttattag

SEQ ID NO: 79 - DNA, 35 bp, Artificial Sequence
  Oligonucleotide primer
gcatggttaccctgcaggggctgctgctgttgctg

SEQ ID NO: 80 - DNA, 37 bp, Artificial Sequence; identical to SEQ ID NO: 36
  Oligonucleotide primer

SEQ ID NO: 81 - DNA, 23 bp, Artificial Sequence
  Binding domain of PTM molecule
acccatcattattaggtcattat

SEQ ID NO: 82 - DNA, 22 bp, Artificial Sequence
  Oligonucleotide primer
gatcaaatctgtcgatccttcc

SEQ ID NO: 83 - DNA, 21 bp, Artificial Sequence
  Oligonucleotide primer
ctgatccacccagtcccatta

SEQ ID NO: 84 - DNA, 22 bp, Artificial Sequence
  Oligonucleotide primer
gactgatccacccagtcccaga

SEQ ID NO: 85 - DNA, 52 bp, Artificial Sequence
  Random sequence inserted to replace 3′ splice site
ccgcggnnnnnnnnnnnnnnnnnnnnnnnngggttccggtaccggcggcttc

SEQ ID NO: 86 - DNA, 71 bp, Artificial Sequence
  Oligonucleotide primer
ttttatccccgtttacagggcggcttcgtctgggactgggtggatcagtcgctgattaaatatgatgaaaa

SEQ ID NO: 87 - DNA, 66 bp, Artificial Sequence
  Oligonucleotide
tttggcgatacgccgaacgatcgccagttctgtatgaacggtctggtctttgccgaccgcacgccg

SEQ ID NO: 88 - DNA, 192 bp, Artificial Sequence
  PTM sequence
acgagcttgctcatgatgatcatgggcgagttagaaccaagtgaaggcaagatcaaacattccggccgcatcagcttttgcagccaattcagttggatcatgcccggtaccatcaaggagaacataatcttcggcgtcagttacgacgagtaccgctatcgctcggtgattaaggcctgtcagttggaggag

SEQ ID NO: 89 - DNA, 25 bp, Artificial Sequence
  Oligonucleotide
gagcaggcaagacgagcttgctcat

SEQ ID NO: 90 - DNA, 28 bp, Artificial Sequence
  Oligonucleotide
gagaacataatcttcggcgtcagttacg

SEQ ID NO: 91 - DNA, 30 bp, Artificial Sequence
  Oligonucleotide
gtcagttggaggaggacatctccaagtttg

SEQ ID NO: 92 - DNA, 192 bp, Artificial Sequence; identical to SEQ ID NO: 88
  PTM exon 10

SEQ ID NO: 93 - DNA, 27 bp, Artificial Sequence
  PTM sequence
aaatatcattggtgtttcttatgatga

SEQ ID NO: 94 - DNA, 30 bp, Artificial Sequence
  Oligonucleotide
ccaactagaagaggacatctccaagtttgc

SEQ ID NO: 95 - DNA, 30 bp, Artificial Sequence
  Oligonucleotide
atgatcatgggcgagttagaaccaagtgag

SEQ ID NO: 96 - DNA, 27 bp, Artificial Sequence
  Oligonucleotide
aaaatatcatctttggtgtttcctatg

SEQ ID NO: 97 - DNA, 27 bp, Artificial Sequence
  Oligonucleotide
ccaactagaagaggacatctccaagtt

SEQ ID NO: 98 - DNA, 21 bp, Artificial Sequence
  5′ Splice site
cgtttacaggtaagtggatcc

SEQ ID NO: 99 - DNA, 27 bp, Artificial Sequence
  3′ Splice site
ctgcagggcggcttcgtctaataatgg

SEQ ID NO: 100 - DNA, 47 bp, Artificial Sequence; identical to SEQ ID NO: 71
  Sequence from trans-splicing domain

SEQ ID NO: 101 - DNA, 1584 bp, Artificial Sequence
  CFTR PTM
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgatcgagaacatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacgagcttgctcatgatgatcatgggcgagttagaaccaagtgaaggcaagatcaaacattccggccgcatcagcttttgcagccaattcagttggatcatgcccggtaccatcaaggagaacataatcttcggcgtcagttacgacgagtaccgctatcgctcggtgattaaggcctgtcagttggaggag

SEQ ID NO: 102 - DNA, 323 bp, Artificial Sequence
  Trans-splicing domain of CFTR PTM
gtaagatatcaccgatatgtgtctaacctgattcgggccttcgatacgctaagatccaccggtcaaaaagttttcacataatttcttacctcttcttgaattcatgctttgatgacgcttctgtatctatattcatcattggaaacaccaatgatattttctttaatggtgcctggcataatcctggaaaactgataacacaatgaaattcttccactgtgcttaattttaccctctgaattctccatttctcccataatcatcattacaactgaactctggaaataaaacccatcattattaactcattatcaaatcacgct

SEQ ID NO: 103 - DNA, 165 bp, Artificial Sequence
  PTM Binding domain
gctagcaataatgacgaagccgcccctcacgctcaggattcacttgcctccaattatcatcctaagcagaagtgtatattcttatttgtaaagattctattaactcatttgattcaaaatatttaaaatacttcctgtttcacctactctgctatgcacccgcgg

SEQ ID NO: 104 - DNA, 225 bp, Artificial Sequence
  Trans-splicing domain of CFTR PTM
aataatgacgaagccgcccctcacgctcaggattcacttgccctccaattatcatcctaagcagaagtgtatattcttatttgtaaagattctattaactcatttgattcaaaatatttaaaatacttcctgtttcacctactctgctatgcacccgcggaacattattataacgttgctcgaatactaactggtacctcttcttttttttttgatatcctgcag

SEQ ID NO: 105 - DNA, 3069 bp, Artificial Sequence
  CFTR PTM sequence
acttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggcttcatcatcatcatcatcattag

SEQ ID NO: 106 - DNA, 131 bp, Artificial Sequence
  Binding domain of mouse factor VIII PTM
ctcgagcttacctgaactaattttttagaatattaaaatcctaagcttttatatctctatccctctatcttttgctctctatccaatttttattaacttagactttaaaaagaaacttatgagaaaaattt

SEQ ID NO: 107 - DNA, 71 bp, Artificial Sequence
  Spacer sequence of PTM
ccgcggaacattattataacgttgctcgaatactaactggtacctcttcttttttttttgatatcctgcag

SEQ ID NO: 108 - DNA, 527 bp, Artificial Sequence
  Chicken beta actin promoter sequences
ccatggtcgacgttagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagttccttttatcgcgaggcggcggcggcggcggccctataaaaagcgaagcgcgcggcggccgggagtcgctgcgacgctgccttcgccccgtgccaacctccgcctcgagcttacctgaactaattttttagaatattaaaatcctaagcttttatactcctatccctctatcttttgctctctatccaatttttattaacttagactttaaaaagaaacttatgagaaaaatttccgcggaacattattataacgttgctcgaatactaactggtacctcttcttttttttttgatatcctgcag

SEQ ID NO: 109 - DNA, 169 bp, Artificial Sequence
  Sequence not included in construct
cgccgcctcgcgccgcccgccccggctctgactgaccgcgttactcccacaggtgagcgggcgggacggcccttctcctccgggctgtaattagcgcttggtttaatcacggcttgtttcttttctgtggctgcgtgaaagccttgaggggctccgggaggaattcgta

SEQ ID NO: 110 - DNA, 42 bp, Artificial Sequence
  F8 PTM sequences
ggagtcgctgcgacgctgccttcgccccgtgccaacctccgc

SEQ ID NO: 111 - DNA, 22 bp, Artificial Sequence
  F8 PTM sequences
ctcgagcaccgatatcgtaact

SEQ ID NO: 112 - DNA, 53 bp, Artificial Sequence
  Exon 26, Flag tag, stop sequences of mouse factor VIII PTM
gaggcccagcagcaatacgactacaaggacgacgatgacaagtgagtttaaac

SEQ ID NO: 113 - DNA, 71 bp, Artificial Sequence; identical to SEQ ID NO: 107
  Spacer sequences of human or canine factor VIII PTM

SEQ ID NO: 114 - DNA, 47 bp, Artificial Sequence; identical to SEQ ID NO: 71
  Branch point and polypyrimidine tract sequences of human papilloma virus PTM

SEQ ID NO: 115 - DNA, 47 bp, Artificial Sequence; identical to SEQ ID NO: 71
  Branch point and polypyrimidine tract of human papilloma virus PTM

SEQ ID NO: 116 - DNA, 80 bp, Artificial Sequence
  Binding domain of human papilloma virus PTM
cagttaatacacctaattaacaaatcacacaacgctttgttgtattgctgttctaatgttgttccatacacactataaca

SEQ ID NO: 117 - DNA, 149 bp, Artificial Sequence
  Binding domain of human papilloma virus PTM
cagttaatacacctaattaacaaatcacacaacgctttgttgtattgctgttctaatgttgttccatacacactataacaataatgtctatactcactaattttagaataaaactttaaacatttatcacatacagcatatcgattccc

SEQ ID NO: 118 - DNA, 35 bp, Artificial Sequence
  Binding domain of human papilloma virus PTM
gatgatctgcaacaagacatacatcgaccggtcca

SEQ ID NO: 119 - DNA, 104 bp, Artificial Sequence
  Binding domain of human papilloma virus PTM
cttcaggacacagtggcttttgacagttaatacacctaattaacaaatcacacaacggtttgttgtattgcagttctatgttgttccatacacactataacaat

SEQ ID NO: 120 - DNA, 18 bp, Artificial Sequence
  Binding domain of human papilloma virus PTM
gatgatctgcaacaagac

SEQ ID NO: 121 - DNA, 99 bp, Artificial Sequence
  Binding domain of human papilloma virus PTM
gacacagtggcttttgacagttaatacacctaattaacaaatcacacaacggtttgttgtattgcagttctaatgttgttccatacacactataacaat

SEQ ID NO: 122 - DNA, 138 bp, Artificial Sequence
  Binding domain of human papilloma virus PTM
gatgatctgcaacaagacatacatcgaccggtccacttcaggacacagtggcttttgacagttaatagacctaattaacaaatcacacaacggtttgttgtattgcagttctaatgttgttccatacacactataaca

SEQ ID NO: 123 - DNA, 89 bp, Artificial Sequence
  Binding domain of human papilloma virus PTM
gatgatctgcaacaagacgacacagtggcttttgacagttaatacacctaattaacaaatcacacaacggtttgttgtattgcagttct

SEQ ID NO: 124 - DNA, 66 bp, Artificial Sequence
  Trans-spliced product
agaatgtgtgtactgcaagcaacagttactgcgacgtgagggcggcttcgtctgggactgggtgga

SEQ ID NO: 125 - DNA, 71 bp, Artificial Sequence
  Trans-spliced product
gtgtactgcaagcaacagttactgcgacgtgagggcggcttcgtctgggactgggtggatcagtcgctgat

SEQ ID NO: 126 - DNA, 500 bp, Artificial Sequence
  Reverse complement of trans-spliced product comprising cystic fibrosis transmembrane regulator-derived sequences and His tag sequences
gtgggagtggcaccttccagggtcaaggaaggcacgggggaggggcaaacaacagatggctggcaactagaaggcacagtcgaggctgatcagcggtttaaacttaagcttggtaccgagctcggatccactagtccagtgtggtggaattctgcagatatccagcacagtggcggccgcctaatgatgatgatgatgatgctcttctagttggcatgctttgatgacgcttctgtatctatattcatcataggaaacaccaaagatgatattttctttaatggtgccaggcataatccaggaaaactgagaacagaatgaaattcttccactgtgcttaattttaccctctgaaggctccagttctcccataatcatcattagaagtgaagtctgcaggaaaaaaaagaagaggtaccagttagtactcgagcaacgttataataatgttccgcggataatgacctaataatgatgggtgggcccgtttaaacgctagc

SEQ ID NO: 127 - DNA, 12 bp, Artificial Sequence
  3′end of trans-spliced product comprising cystic fibrosis transmembrane regulator-derived sequences and His tag sequences
gctagcgtttaa

SEQ ID NO: 128 - DNA, 12 bp, Artificial Sequence
  5′end of trans-spliced product comprising cystic fibrosis transmembrane regulator-derived sequences and His tag sequences
tgccactcccac